SEQUENCE LISTING (187 sequences)

SEQ ID NO: 1 - protein, 20 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGEX

SEQ ID NO: 2 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGEXAKK

SEQ ID NO: 3 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKSKKKFGKAXVGEXAKK

SEQ ID NO: 4 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHKKKKFGKAXVGEXAKK

SEQ ID NO: 5 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXAKK

SEQ ID NO: 6 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGSFXHKKKKFGKAXVGEXAKK

SEQ ID NO: 7 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHNKKKFGKAXVGEXAKK

SEQ ID NO: 8 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHQKKKFGKAXVGEXAKK

SEQ ID NO: 9 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHTKKKFGKAXVGEXAKK

SEQ ID NO: 10 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHYKKKFGKAXVGEXAKK

SEQ ID NO: 11 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVWEXAKK

SEQ ID NO: 12 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVVEXAKK

SEQ ID NO: 13 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVLEXAKK

SEQ ID NO: 14 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVYEXAKK

SEQ ID NO: 15 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVFEXAKK

SEQ ID NO: 16 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVTEXAKK

SEQ ID NO: 17 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGDXAKK

SEQ ID NO: 18 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGQXAKK

SEQ ID NO: 19 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGNXAKK

SEQ ID NO: 20 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKSKKKFGKAXVVEXAKK

SEQ ID NO: 21 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKSKKKFGKAXVFEXAKK

SEQ ID NO: 22 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHKKKKFGKAXVVEXAKK

SEQ ID NO: 23 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHKKKKFGKAXVFEXAKK

SEQ ID NO: 24 - protein, 23 aa, synthetic construct
  VARIANT 2: note = X can be any natural or artificial amino acid
  SITE 4: note = Dbu
  VARIANT 6: note = X can be any natural or artificial amino acid
  SITE 7..11: note = Dbu
  VARIANT 14: note = Dbu
  VARIANT 16: note = X can be any natural or artificial amino acid
  VARIANT 20: note = X can be any natural or artificial amino acid
  SITE 22..23: note = Dbu
GXGXFXXXXXXFGXAXVGEXAXX

SEQ ID NO: 25 - protein, 23 aa, synthetic construct
  VARIANT 2: note = X can be any natural or artificial amino acid
  SITE 4: note = Orn
  VARIANT 6: note = X can be any natural or artificial amino acid
  SITE 7..11: note = Orn
  SITE 14: note = Orn
  VARIANT 16: note = X can be any natural or artificial amino acid
  VARIANT 20: note = X can be any natural or artificial amino acid
  SITE 22..23: note = Orn
GXGXFXXXXXXFGXAXVGEXAXX

SEQ ID NO: 26 - protein, 23 aa, synthetic construct
  VARIANT 2: note = X can be any natural or artificial amino acid
  SITE 4: note = Dpr
  VARIANT 6: note = X can be any natural or artificial amino acid
  SITE 7..11: note = Dpr
  SITE 14: note = Dpr
  VARIANT 16: note = X can be any natural or artificial amino acid
  VARIANT 20: note = X can be any natural or artificial amino acid
  SITE 22..23: note = Dpr
GXGXFXXXXXXFGXAXVGEXAXX

SEQ ID NO: 27 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXAKKGGE

SEQ ID NO: 28 - protein, 21 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXK

SEQ ID NO: 29 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 5: note = D-phenylalanine
  SITE 12: note = D-phenylalanine
GXGKFXHKKKKFGKAXVVEXAKK

SEQ ID NO: 30 - protein, 23 aa, synthetic construct
  VARIANT 2: note = X can be any natural or artificial amino acid
  MOD_RES 5: note = L-4-fluoro phenylalanine
  MOD_RES 12: note = L-4-fluoro phenylalanine
  VARIANT 6: note = X can be any natural or artificial amino acid
  VARIANT 16: note = X can be any natural or artificial amino acid
  VARIANT 20: note = X can be any natural or artificial amino acid
GXGKFXHKKKKFGKAXVVEXAKK

SEQ ID NO: 31 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  MOD_RES 5: note = L-4-methyl phenylalanine
  MOD_RES 12: note = L-4-methyl phenylalanine
GXGKFXHKKKKFGKAXVVEXAKK

SEQ ID NO: 32 - protein, 23 aa, synthetic construct
  MOD_RES 5: note = L-4-trifluoromethyl phenylalanine
  MOD_RES 12: note = L-4-trifluoromethyl phenylalanine
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHKKKKFGKAXVVEXAKK

SEQ ID NO: 33 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  MOD_RES 5: note = L-4-t-butyl phenylalanine
  MOD_RES 12: note = L-4-t-butyl phenylalanine
GXGKFXHKKKKFGKAXVVEXAKK

SEQ ID NO: 34 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVVEXAKKGGE

SEQ ID NO: 35 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVFEXAKKGGE

SEQ ID NO: 36 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 18: note = D-Phenylalanine
GXGKFXKKKKKFGKAXVFEXAKKGGE

SEQ ID NO: 37 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  MOD_RES 18: note = L-4-trifluoromethyl phenylalanine
GXGKFXKKKKKFGKAXVFEXAKKGGE

SEQ ID NO: 38 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  MOD_RES 18: note = L-4-t-butyl phenylalanine
GXGKFXKKKKKFGKAXVFEXAKKGGE

SEQ ID NO: 39 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dbu
GXGXFXKKKKKFGKAXVGEXAKK

SEQ ID NO: 40 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 8: note = Dbu
GXGKFXKXKKKFGKAXVGEXAKK

SEQ ID NO: 41 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 14: note = Dbu
GXGKFXKKKKKFGXAXVGEXAKK

SEQ ID NO: 42 - protein, 23 aa, synthetic construct
  SITE 9: note = Dpr
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKXKKFGKAXVGEXAKK

SEQ ID NO: 43 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dbu
GXGKFXXKKKKFGKAXVGEXAKK

SEQ ID NO: 44 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 9: note = Dbu
GXGKFXKKXKKFGKAXVGEXAKK

SEQ ID NO: 45 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 10: note = Dbu
GXGKFXKKKXKFGKAXVGEXAKK

SEQ ID NO: 46 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 22: note = Dbu
GXGKFXKKKKKFGKAXVGEXAXK

SEQ ID NO: 47 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dpr
GXGXFXKKKKKFGKAXVGEXAKK

SEQ ID NO: 48 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFGKAXVGEXAKK

SEQ ID NO: 49 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 8: note = Dpr
GXGKFXKXKKKFGKAXVGEXAKK

SEQ ID NO: 50 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 10: note = Dpr
GXGKFXKKKXKFGKAXVGEXAKK

SEQ ID NO: 51 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 11: note = Dpr
GXGKFXKKKKXFGKAXVGEXAKK

SEQ ID NO: 52 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 14: note = Dpr
GXGKFXKKKKKFGXAXVGEXAKK

SEQ ID NO: 53 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 22: note = Dpr
GXGKFXKKKKKFGKAXVGEXAXK

SEQ ID NO: 54 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 23: note = Dpr
GXGKFXKKKKKFGKAXVGEXAKX

SEQ ID NO: 55 - protein, 25 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXAKKGE

SEQ ID NO: 56 - protein, 27 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXAKKGGGE

SEQ ID NO: 57 - protein, 27 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXAKKGGEE

SEQ ID NO: 58 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
WXGKFXHSKKKFGKAXVGEXAKK

SEQ ID NO: 59 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXWKFXHSKKKFGKAXVGEXAKK

SEQ ID NO: 60 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGWFXHSKKKFGKAXVGEXAKK

SEQ ID NO: 61 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKWXHSKKKFGKAXVGEXAKK

SEQ ID NO: 62 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXWSKKKFGKAXVGEXAKK

SEQ ID NO: 63 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHWKKKFGKAXVGEXAKK

SEQ ID NO: 64 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKWWKAXVGEXAKK

SEQ ID NO: 65 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKWXVGEXAKK

SEQ ID NO: 66 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXWGEXAKK

SEQ ID NO: 67 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVWEXAKK

SEQ ID NO: 68 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGWXAKK

SEQ ID NO: 69 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFGKAXVGEXAKW

SEQ ID NO: 70 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKWGKAXVGEXAKK

SEQ ID NO: 71 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXHSKKKFWKAXVGEXAKK

SEQ ID NO: 72 - protein, 19 aa, synthetic construct
  VARIANT 2: note = X can be any natural or artificial amino acid
  VARIANT 6: note = X can be any natural or artificial amino acid
  VARIANT 12: note = X can be any natural or artificial amino acid
  VARIANT 16: note = X can be any natural or artificial amino acid
GXGKFXKKKKKXVGEXAKK

SEQ ID NO: 73 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXFSKXKGKKIKNLXISGXKG

SEQ ID NO: 74 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
GXKSKXKGKKIKNLXISGXKG

SEQ ID NO: 75 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXFSKXKGKKIKNLXISKXKG

SEQ ID NO: 76 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXFSKXKGKKIKNLXISGXKK

SEQ ID NO: 77 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXKSKXKGKKIKNLXISKXKG

SEQ ID NO: 78 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXKVKXKGKKIKNLXISGXKG

SEQ ID NO: 79 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXKSKXKVKKIKNLXISGXKG

SEQ ID NO: 80 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXKSKXKGKKVKNLXISGXKG

SEQ ID NO: 81 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
GXKSKXAGKKLKNLXISGXKN

SEQ ID NO: 82 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXFSKXAGKKIKNLXISGXKN

SEQ ID NO: 83 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXKSKXAGKKIKNLXISGXKN

SEQ ID NO: 84 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXKSKXAGKKLKNLXISGXKG

SEQ ID NO: 85 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
GXFSKXAGKKLKNLXISGXKN

SEQ ID NO: 86 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
  SITE 5: note = Dbu
  SITE 7: note = Dbu
  SITE 9..10: note = Dbu
  SITE 12: note = Dbu
  SITE 20: note = Dbu
GXFSXXXGXXIXNLXISGXXG

SEQ ID NO: 87 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
  SITE 5: note = Orn
  SITE 7: note = Orn
  SITE 9..10: note = Orn
  SITE 12: note = Orn
  SITE 20: note = Orn
GXFSXXXGXXIXNLXISGXXG

SEQ ID NO: 88 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
  SITE 5: note = Dpr
  SITE 7: note = Dpr
  SITE 9..10: note = Dpr
  SITE 12: note = Dpr
  SITE 20: note = Dpr
GXFSXXXGXXIXNLXISGXXG

SEQ ID NO: 89 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXKSKXKGKKIKNLXISGXKN

SEQ ID NO: 90 - protein, 21 aa, synthetic construct
  VARIANT 2: note = X can be any natural or artificial amino acid
  VARIANT 6: note = X can be any natural or artificial amino acid
  SITE 15: note = X can be any natural or artificial amino acid
  VARIANT 19: note = X can be any natural or artificial amino acid
  SITE 11: note = L-4-t-butyl phenylalanine
GXKSKXKGKKFKNLXISGXKN

SEQ ID NO: 91 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
  SITE 14: note = L-4-t-butyl phenylalanine
GXKSKXKGKKIKNFXISGXKN

SEQ ID NO: 92 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
  SITE 16: note = L-4-t-butyl phenylalanine
GXKSKXKGKKIKNLXFSGXKN

SEQ ID NO: 93 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXKSKXKGKKIKNLXISVXKN

SEQ ID NO: 94 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXKSKXKGKKIKNLXISFXKN

SEQ ID NO: 95 - protein, 22 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXKSKXKGKKIKNLXISFXKNE

SEQ ID NO: 96 - protein, 25 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXKSKXKGKKIKNLXISFXKNGGGE

SEQ ID NO: 97 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
KXFSKXKGKKIKNLXISGXKG

SEQ ID NO: 98 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXFKKXKGKKIKNLXISGXKG

SEQ ID NO: 99 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXFSKXKKKKIKNLXISGXKG

SEQ ID NO: 100 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXFSKXKGKKKKNLXISGXKG

SEQ ID NO: 101 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXFSKXKGKKIKKLXISGXKG

SEQ ID NO: 102 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXFSKXKGKKIKNKXISGXKG

SEQ ID NO: 103 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXFSKXKGKKIKNLXKSGXKG

SEQ ID NO: 104 - protein, 21 aa, synthetic construct
  VARIANT 2: note = X can be any natural or artificial amino acid
  VARIANT 6: note = X can be any natural or artificial amino acid
  VARIANT 15: note = X can be any natural or artificial amino acid
  VARIANT 19: note = X can be any natural or artificial amino acid
GXFSKXKGKKIKNLXIKGXKG

SEQ ID NO: 105 - protein, 21 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXFSKXKGKKIKNLLXSGLXG

SEQ ID NO: 106 - protein, 21 aa, synthetic construct
  VARIANT 2: note = X can be any natural or artificial amino acid
  VARIANT 6: note = X can be any natural or artificial amino acid
  VARIANT 14: note = X can be any natural or artificial amino acid
  VARIANT 18: note = X can be any natural or artificial amino acid
GXFSKXKGKKIKNXLISXLKG

SEQ ID NO: 107 - protein, 21 aa, synthetic construct
  VARIANT 2: note = X can be any natural or artificial amino acid
  VARIANT 6: note = X can be any natural or artificial amino acid
  VARIANT 13: note = X can be any natural or artificial amino acid
  VARIANT 17: note = X can be any natural or artificial amino acid
GXFSKXKGKKIKXLLIXGLKG

SEQ ID NO: 108 - protein, 21 aa, synthetic construct
  VARIANT 2: note = X can be any natural or artificial amino acid
  VARIANT 6: note = X can be any natural or artificial amino acid
  VARIANT 11: note = X can be any natural or artificial amino acid
  VARIANT 15: note = X can be any natural or artificial amino acid
GXFSKXKGKKXKNLXISGLKG

SEQ ID NO: 109 - protein, 21 aa, synthetic construct
  VARIANT 7: note = X can be any natural or artificial amino acid
  VARIANT 11: note = X can be any natural or artificial amino acid
GIFSKLXGKKXKNLLISGLKG

SEQ ID NO: 110 - protein, 17 aa, synthetic construct
  VARIANT 2: note = X can be any natural or artificial amino acid
  VARIANT 6: note = X can be any natural or artificial amino acid
  VARIANT 11: note = X can be any natural or artificial amino acid
  VARIANT 15: note = X can be any natural or artificial amino acid
GXFSKXKGKKXISGXKG

SEQ ID NO: 111 - protein, 21 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXFSKXKGKKIKNLLXSGKXG

SEQ ID NO: 112 - protein, 21 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXFSKXKGKKIKNLLXSGLXK

SEQ ID NO: 113 - protein, 24 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXFSKXKGKKIKNLLXSGLXKGGE

SEQ ID NO: 114 - protein, 21 aa, synthetic construct
  VARIANT 7: note = X can be any natural or artificial amino acid
  VARIANT 11: note = X can be any natural or artificial amino acid
GIFSKLXGKKXKNLKISGLKG

SEQ ID NO: 115 - protein, 21 aa, synthetic construct
  VARIANT 1: note = X can be any natural or artificial amino acid
  VARIANT 5: note = X can be any natural or artificial amino acid
XIFSXLAGKKIKNLLISGLKG

SEQ ID NO: 116 - protein, 21 aa, synthetic construct
  VARIANT 2: note = X can be any natural or artificial amino acid
  VARIANT 6: note = X can be any natural or artificial amino acid
GXFSKXAGKKIKNLLISGLKG

SEQ ID NO: 117 - protein, 21 aa, synthetic construct
  VARIANT 3: note = X can be any natural or artificial amino acid
  VARIANT 7: note = X can be any natural or artificial amino acid
GIXSKLXGKKIKNLLISGLKG

SEQ ID NO: 118 - protein, 21 aa, synthetic construct
  VARIANT 4: note = X can be any natural or artificial amino acid
  VARIANT 8: note = X can be any natural or artificial amino acid
GIFXKLAXKKIKNLLISGLKG

SEQ ID NO: 119 - protein, 21 aa, synthetic construct
  VARIANT 5: note = X can be any natural or artificial amino acid
  VARIANT 9: note = X can be any natural or artificial amino acid
GIFSXLAGXKIKNLLISGLKG

SEQ ID NO: 120 - protein, 21 aa, synthetic construct
  VARIANT 6: note = X can be any natural or artificial amino acid
  VARIANT 10: note = X can be any natural or artificial amino acid
GIFSKXAGKXIKNLLISGLKG

SEQ ID NO: 121 - protein, 21 aa, synthetic construct
  VARIANT 7: note = X can be any natural or artificial amino acid
  VARIANT 11: note = X can be any natural or artificial amino acid
GIFSKLXGKKXKNLLISGLKG

SEQ ID NO: 122 - protein, 21 aa, synthetic construct
  VARIANT 8: note = X can be any natural or artificial amino acid
  VARIANT 12: note = X can be any natural or artificial amino acid
GIFSKLAXKKIXNLLISGLKG

SEQ ID NO: 123 - protein, 21 aa, synthetic construct
  VARIANT 9: note = X can be any natural or artificial amino acid
  VARIANT 13: note = X can be any natural or artificial amino acid
GIFSKLAGXKIKXLLISGLKG

SEQ ID NO: 124 - protein, 21 aa, synthetic construct
  VARIANT 10: note = X can be any natural or artificial amino acid
  VARIANT 14: note = X can be any natural or artificial amino acid
GIFSKLAGKXIKNXLISGLKG

SEQ ID NO: 125 - protein, 21 aa, synthetic construct
  VARIANT 11: note = X can be any natural or artificial amino acid
  PEPTIDE 15: note = 15
GIFSKLAGKKXKNLXISGLKG

SEQ ID NO: 126 - protein, 21 aa, synthetic construct
  VARIANT 12: note = X can be any natural or artificial amino acid
  VARIANT 16: note = X can be any natural or artificial amino acid
GIFSKLAGKKIXNLLXSGLKG

SEQ ID NO: 127 - protein, 21 aa, synthetic construct
  VARIANT 13: note = X can be any natural or artificial amino acid
  VARIANT 17: note = X can be any natural or artificial amino acid
GIFSKLAGKKIKXLLIXGLKG

SEQ ID NO: 128 - protein, 21 aa, synthetic construct
  VARIANT 14: note = X can be any natural or artificial amino acid
  VARIANT 18: note = X can be any natural or artificial amino acid
GIFSKLAGKKIKNXLISXLKG

SEQ ID NO: 129 - protein, 21 aa, synthetic construct
  VARIANT 15: note = X can be any natural or artificial amino acid
  VARIANT 19: note = X can be any natural or artificial amino acid
GIFSKLAGKKIKNLXISGXKG

SEQ ID NO: 130 - protein, 21 aa, synthetic construct
  VARIANT 16: note = X can be any natural or artificial amino acid
  VARIANT 20: note = X can be any natural or artificial amino acid
GIFSKLAGKKIKNLLXSGLXG

SEQ ID NO: 131 - protein, 21 aa, synthetic construct
  VARIANT 17: note = X can be any natural or artificial amino acid
  VARIANT 21: note = X can be any natural or artificial amino acid
GIFSKLAGKKIKNLLIXGLKX

SEQ ID NO: 132 - protein, 21 aa, synthetic construct
  VARIANT 1: note = X can be any natural or artificial amino acid
  VARIANT 8: note = X can be any natural or artificial amino acid
XIFSKLAXKKIKNLLISGLKG

SEQ ID NO: 133 - protein, 21 aa, synthetic construct
  VARIANT 2: note = X can be any natural or artificial amino acid
  VARIANT 9: note = X can be any natural or artificial amino acid
GXFSKLAGXKIKNLLISGLKG

SEQ ID NO: 134 - protein, 21 aa, synthetic construct
  VARIANT 3: note = X can be any natural or artificial amino acid
  VARIANT 10: note = X can be any natural or artificial amino acid
GIXSKLAGKXIKNLLISGLKG

SEQ ID NO: 135 - protein, 21 aa, synthetic construct
  VARIANT 4: note = X can be any natural or artificial amino acid
  VARIANT 11: note = X can be any natural or artificial amino acid
GIFXKLAGKKXKNLLISGLKG

SEQ ID NO: 136 - protein, 21 aa, synthetic construct
  VARIANT 5: note = X can be any natural or artificial amino acid
  VARIANT 12: note = X can be any natural or artificial amino acid
GIFSXLAGKKIXNLLISGLKG

SEQ ID NO: 137 - protein, 21 aa, synthetic construct
  VARIANT 6: note = X can be any natural or artificial amino acid
  VARIANT 13: note = X can be any natural or artificial amino acid
GIFSKXAGKKIKXLLISGLKG

SEQ ID NO: 138 - protein, 21 aa, synthetic construct
  VARIANT 8: note = X can be any natural or artificial amino acid
  VARIANT 15: note = X can be any natural or artificial amino acid
GIFSKLAXKKIKNLXISGLKG

SEQ ID NO: 139 - protein, 21 aa, synthetic construct
  VARIANT 9: note = X can be any natural or artificial amino acid
  VARIANT 16: note = X can be any natural or artificial amino acid
GIFSKLAGXKIKNLLXSGLKG

SEQ ID NO: 140 - protein, 21 aa, synthetic construct
  VARIANT 10: note = X can be any natural or artificial amino acid
  VARIANT 17: note = X can be any natural or artificial amino acid
GIFSKLAGKXIKNLLIXGLKG

SEQ ID NO: 141 - protein, 21 aa, synthetic construct
  VARIANT 11: note = X can be any natural or artificial amino acid
  VARIANT 18: note = X can be any natural or artificial amino acid
GIFSKLAGKKXKNLLISXLKG

SEQ ID NO: 142 - protein, 21 aa, synthetic construct
  VARIANT 12: note = X can be any natural or artificial amino acid
  VARIANT 19: note = X can be any natural or artificial amino acid
GIFSKLAGKKIXNLLISGXKG

SEQ ID NO: 143 - protein, 21 aa, synthetic construct
  VARIANT 13: note = X can be any natural or artificial amino acid
  VARIANT 20: note = X can be any natural or artificial amino acid
GIFSKLAGKKIKXLLISGLXG

SEQ ID NO: 144 - protein, 21 aa, synthetic construct
  VARIANT 14: note = X can be any natural or artificial amino acid
  VARIANT 21: note = X can be any natural or artificial amino acid
GIFSKLAGKKIKNXLISGLKX

SEQ ID NO: 145 - protein, 21 aa, synthetic construct
  VARIANT 7: note = X can be any natural or artificial amino acid
  VARIANT 14: note = X can be any natural or artificial amino acid
GIFSKLXGKKIKNXLISGLKG

SEQ ID NO: 146 - protein, 21 aa, synthetic construct
  VARIANT 7: note = X can be any natural or artificial amino acid
  VARIANT 11: note = X can be any natural or artificial amino acid
  VARIANT 17: note = X can be any natural or artificial amino acid
  VARIANT 21: note = X can be any natural or artificial amino acid
GIFSKLXGKKXKNLLIXGLKX

SEQ ID NO: 147 - protein, 23 aa, synthetic construct
  VARIANT 7: note = X can be any natural or artificial amino acid
  VARIANT 11: note = X can be any natural or artificial amino acid
GIFSKLXGKKXKNLLISGLKGGG

SEQ ID NO: 148 - protein, 21 aa, synthetic construct
  VARIANT 14: note = X can be any natural or artificial amino acid
  VARIANT 18: note = X can be any natural or artificial amino acid
KALETLRRVGDGVXRNHXTAF

SEQ ID NO: 149 - protein, 16 aa, synthetic construct
  VARIANT 9: note = X can be any natural or artificial amino acid
RWPQXILDXHVRRVWR

SEQ ID NO: 150 - protein, 24 aa, synthetic construct
  VARIANT 14: note = X can be any natural or artificial amino acid
  VARIANT 18: note = X can be any natural or artificial amino acid
LSQEQLEHRERSLXTLRXIQRBLF

SEQ ID NO: 151 - skipped

SEQ ID NO: 152 - protein, 14 aa, synthetic construct
  SITE 4: note = R-2-(7'-octenyl) alanine
  SITE 11: note = S-2-(4'-pentenyl) alanine
LTFXEYWAQLXSAA

SEQ ID NO: 153 - protein, 23 aa, Xenopus laevis
GIGKFLHSAGKFGKAFVGEIMKS

SEQ ID NO: 154 - protein, 23 aa, Xenopus laevis
GIGKFLHSAKKFGKAFVGEIMNS

SEQ ID NO: 155 - protein, 46 aa, synthetic construct
GIFSKLAGKKIKNLLISGLKNVGKEVGMDVVRTGIDIAGCKIKGEC

SEQ ID NO: 156 - protein, 4 aa, synthetic construct
GGGE

SEQ ID NO: 157 - protein, 4 aa, synthetic construct
GGEE

SEQ ID NO: 158 - protein, 6 aa, synthetic construct
GGSGGS

SEQ ID NO: 159 - skipped

SEQ ID NO: 160 - protein, 4 aa, synthetic construct
GGFG

SEQ ID NO: 161 - protein, 14 aa, synthetic construct
  VARIANT 4: note = X can be any natural or artificial amino acid
  VARIANT 11: note = X can be any natural or artificial amino acid
LTFXEYWAQLXSAA

SEQ ID NO: 162 - protein, 17 aa, synthetic construct
  VARIANT 4: note = X can be any natural or artificial amino acid
  VARIANT 11: note = X can be any natural or artificial amino acid
LTFXEYWAQLXSAAGGG

SEQ ID NO: 163 - protein, 17 aa, synthetic construct
  VARIANT 4: note = X can be any natural or artificial amino acid
  VARIANT 11: note = X can be any natural or artificial amino acid
LTFXEYWAQLXSAAPEG

SEQ ID NO: 164 - protein, 14 aa, synthetic construct
LTFREYWAQLSSAA

SEQ ID NO: 165 - protein, 17 aa, synthetic construct
LTFREYWAQLSSAAGGG

SEQ ID NO: 166 - protein, 14 aa, synthetic construct
LTFREYWAQLSSAA

SEQ ID NO: 167 - protein, 24 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dpr
  SITE 7..11: note = Dpr
  SITE 14: note = Dpr
  SITE 22..23: note = Dpr
GXGXFXXXXXXFGXAXVGEXAXXG

SEQ ID NO: 168 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXAKKGGQ

SEQ ID NO: 169 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dpr
  SITE 7..11: note = Dpr
  SITE 14: note = Dpr
GXGXFXXXXXXFGXAXVGEXAKK

SEQ ID NO: 170 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dbu
GXGKFXXKKKKFGKAXVGEXAKK

SEQ ID NO: 171 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFGKAXVGEXAKKGGE

SEQ ID NO: 172 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 9: note = Dpr
GXGKFXKKXKKFGKAXVGEXAKKGGE

SEQ ID NO: 173 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
  SITE 9: note = Dpr
GXGKFXXKXKKFGKAXVGEXAKK

SEQ ID NO: 174 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
  SITE 9: note = Dpr
GXGKFXXKXKKFGKAXVGEXAKKGGE

SEQ ID NO: 175 - protein, 23 aa, synthetic construct
  SITE 1: note = L-propargyl glycine
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 4: note = Dpr
  SITE 7..11: note = Dpr
  SITE 14: note = Dpr
  SITE 22..23: note = Dpr
JXGXFXXXXXXFGXAXVGEXAXX

SEQ ID NO: 176 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXSKKKFGKAXVGEXAKK

SEQ ID NO: 177 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFSKAXVGEXAKK

SEQ ID NO: 178 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFGKSXVGEXAKK

SEQ ID NO: 179 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFGKAXVSEXAKK

SEQ ID NO: 180 - protein, 23 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 7: note = Dpr
GXGKFXXKKKKFGKAXVGEXSKK

SEQ ID NO: 181 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKSKKKFGKAXVGEXAKKGGE

SEQ ID NO: 182 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFSKAXVGEXAKKGGE

SEQ ID NO: 183 - protein, 26 aa, synthetic construct
  VARIANT 2: note = X can be any natural or artificial amino acid
  UNSURE 6: note = X can be any natural or artificial amino acid
  VARIANT 16: note = X can be any natural or artificial amino acid
  VARIANT 20: note = X can be any natural or artificial amino acid
GXGKFXKKKKKFGKSXVGEXAKKGGE

SEQ ID NO: 184 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVSEXAKKGGE

SEQ ID NO: 185 - protein, 26 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
GXGKFXKKKKKFGKAXVGEXSKKGGE

SEQ ID NO: 186 - protein, 27 aa, synthetic construct
  VARIANT 2: note = X can be any natural or artificial amino acid
  VARIANT 6: note = X can be any natural or artificial amino acid
  VARIANT 16: note = X can be any natural or artificial amino acid
  VARIANT 20: note = X can be any natural or artificial amino acid
  SITE 27: note = L-propargyl glycine
GXGKFXKKKKKFGKAXVGEXAKKGGEJ

SEQ ID NO: 187 - protein, 4 aa, synthetic construct
GGGC

(19) United States
(12) Patent Application Publication
(10) Pub. No.: US 2026/0263623 A1
(43) Pub. Date: Sep. 10, 2026

(54) STAPLED PEPTIDE-ANTIBODY CONJUGATES (SPACS) AND USES THEREOF

(71) Applicant: Lytica Therapeutics, Inc., Boston, MA (US)

(72) Inventors: Rida Mourtada, Boston, MA (US); Daniel T. Cohen, Boston, MA (US); John Ernest Vallarta Bajacan, Boston, MA (US)

(73) Assignee: Lytica Therapeutics, Inc., Boston, MA (US)

(21) Appl. No.: 18/716,717

(22) PCT Filed: Dec. 9, 2022
(86) PCT No.: PCT/US2022/052358

§ 371 Date: Jun. 5, 2024

Related U.S. Application Data
(60) Provisional application No. 63/288,498, filed on Dec. 10, 2021.
(60) Provisional application No. 63/321,968, filed on Mar. 21, 2022.
(60) Provisional application No. 63/353,275, filed on Jun. 17, 2022.

(51) Int. Cl. A61K 47/68; A61P 35/00

(52) U.S. Cl. CPC A61K 47/6855; A61K 47/6811; A61K 47/6849; A61K 47/6889; A61P 35/00

(57) ABSTRACT

Provided herein are stapled peptide-antibody conjugates (SPACs) comprising a stapled peptide conjugated to an antibody or antigen-binding fragment thereof. In certain embodiments, the stapled peptide is conjugated to the antibody or antigen-binding fragment thereof via a linker. In certain embodiments, the SPACs provided herein can be used to deliver stapled peptides to cells (e.g., cancer cells) with relatively high selectivity and relatively low off-target toxicity. Also provided herein are pharmaceutical compositions and kits comprising the SPACs described herein, methods of using the same (e.g., to treat cancer and/or inhibit tumor growth in a subject), and methods of preparing the same.

### RELATED APPLICATIONS

This application claims priority under 35 U.S.C. § 119(e) to United States Provisional Patent Applications, U.S. Ser. No. 63/288,498, filed Dec. 10, 2021; U.S. Ser. No. 63/321,968, filed Mar. 21, 2022; and U.S. Ser. No. 63/353,275, filed Jun. 17, 2022, the entire contents of which is incorporated herein by reference, BACKGROUND

Antibody-drug conjugates (ADCs) are a unique class of oncology drugs that can carry highly cytotoxic payloads directly into cells (e.g., cancer cells). This advance in precision medicine has allowed for the use of very potent chemotherapeutics that previously had very narrow therapeutic windows. ADCs are described in, e.g., Drago et al., Nature Reviews Clinical Oncology, 2021, vol. 18, 327-344; Baah et al., Molecules, 2021, 26(10), 2943; Khongorzul et al., Mol. Cancer Res., 2020, 18(1):3-19; and Beck et al., Nature Reviews Drug Discovery, 2017, vol. 16, 315-337.

Interestingly, due to the high potency of their cargo, ADCs can be used to treat cancer patients that have been heavily pretreated with other chemotherapeutics, including chemotherapeutics in the same therapeutic class, and even the same unconjugated antibody. However, despite major advances in the field, some critical liabilities of ADCs still need to be resolved. One of these issues is the non-specific toxicity of the ADC cargo due to, e.g., linker instability. Since these cargos tend to be highly cytotoxic, they can damage healthy tissue when they are unintentionally released outside of a tumor cell. Efforts to resolve this problem have been mainly focused on creating new linkers with higher stability and release selectivity.

### SUMMARY OF THE INVENTION

Described herein is a new class of therapeutic agents that resolves many of the issues with current antibody-drug conjugates (ADCs) and expands the payload beyond the current, highly cytotoxic agents. Relying on a unique class of peptides known as stapled peptides, the conjugates described herein have distinct advantages without many of the liabilities of current ADCs.

Stapled peptides are synthetic peptides comprising at least two unnatural amino acids connected via a crosslink (i.e., “staple”). For example, a stapled peptide can comprise at least two unnatural olefin-bearing amino acids cyclized via metathesis to form an internal hydrocarbon crosslink (i.e., “hydrocarbon staple”). This process of cyclization, or “stapling,” can help fold the peptide in an alpha-helical confirmation or other secondary structure, recapturing the natural three-dimensional orientation of protein structures to mimic their biological functions. Examples of stapled peptide technology can be found in, e.g., International PCT Application Publication Nos. WO 2017/004591, published Jan. 5, 2017; WO 2019/018499, published Jan. 24, 2019; and WO 2021/126827, published Jun. 24, 2021, the entire contents of each of which are incorporated herein by reference. See also, e.g., Mourtada et al., Nature Biotechnology, 2019, vol. 37, 1186-1197, the entire contents of which is incorporated herein by reference.

Since their development in the early 2000s, stapled peptides have been shown to be able to disrupt protein-protein interactions (PPIs), both extracellularly and intracellularly. Currently, Aileron Therapeutics has a stapled peptide drug candidate, ALRN-6924, in Phase 2 clinical trials as a prophylactic treatment to prevent chemotherapy-induced neutropenia. Although stapled peptides themselves are promising therapeutic agents, a challenge for some development efforts has been the inability to deliver stapled peptides intracellularly without triggering lysis of off-target cells. This is because many stapled peptides have exhibited low cell penetrance.

In the context of antibody conjugates, however, the low cell penetrance of some stapled peptides can be an asset rather than a liability. By conjugating a stapled peptide to an antibody or antigen-binding fragment thereof, one can take advantage of the conjugate's internalization to deliver a cargo/payload (i.e., a stapled peptide) into a cell that would otherwise be inactive or have low activity outside of the cell. Similarly, the ability of antibodies to aggregate payloads at the plasma membrane of receptor-positive cells may allow for targeted delivery of otherwise non-specifically lytic stapled peptides. Leveraging the unique properties of stapled peptides in these systems allows for the delivery of therapeutic agents with high potency, but without off-target toxicity of previous ADCs.

Provided herein are stapled peptide-antibody conjugates (SPACs) comprising a stapled peptide conjugated to an antibody or antigen-binding fragment thereof. In certain embodiments, the stapled peptide is conjugated to the antibody or antigen-binding fragment thereof via a linker. In certain embodiments, the SPACs provided herein can be used to deliver stapled peptides to cells (e.g., cancer cells, bacterial cells) with relatively high selectivity and/or relatively low off-target toxicity.

SPACs provided herein comprise an antibody or antigen-binding fragment thereof. Antibodies (including intact antibodies and antigen-binding fragments), variants, and derivatives thereof include, but are not limited to, polyclonal, monoclonal, multispecific, human, humanized, primatized, or chimeric antibodies, heteroconjugate antibodies (e.g., bi-tri- and quad-specific antibodies, diabodies, triabodies, and tetrabodies), single-domain antibodies (sdAb), epitope-binding fragments (e.g., Fab, Fab′, and F(ab′)2, Fd, Fvs, single-chain Fvs (scFv), rlgG, single-chain antibodies, disulfide-linked Fvs (sdFv), fragments containing either a VL or VH domain, fragments produced by an Fab expression library), and anti-idiotypic (anti-Id) antibodies. Fab and F(ab′)2 fragments, for example, lack the Fc fragment of an intact antibody. Antibody molecules of the conjugates can be of any type (e.g., IgG, IgE, IgM, IgD, IgA, and IgY), class (e.g., IgG1, IgG2, IgG3, IgG4, IgA1 and IgA2) or subclass of immunoglobulin molecule.

In certain embodiments, the antibody is a monoclonal antibody (mAb) or antigen-binding fragment thereof. In certain embodiments, the antibody is an anti-cancer antibody or antigen-binding fragment thereof. In certain embodiments, the antibody or antigen-binding fragment thereof is directed against a target antigen expressed on a cancer cell (e.g., expressed specifically on a cancer cell). In certain embodiments, the antibody is a homolog of an antibody or antigen-binding fragment thereof described herein.

In certain embodiments, the antibody is an antibody-drug conjugate (ADC), or antigen binding fragment thereof. In such embodiments, the SPAC comprises an antibody or antigen binding fragment thereof conjugated to dual payloads: (i) a stapled peptide or pharmaceutically acceptable salt thereof; and (ii) a second agent (i.e., the “drug” component of the antibody-drug conjugate).

The stapled peptide component of the SPAC may be any stapled peptide (e.g., any singly stapled, doubly stapled, multiply stapled, or stitched peptide). In certain embodiments, the stapled peptide is a stapled anti-cancer peptide. In certain embodiments, the stapled peptide is a stapled antimicrobial peptide (StAMP). In certain embodiments, the stapled peptide is a stapled Magainin peptide (e.g., stapled Magainin II peptide). In certain embodiments, the stapled peptide is a stapled Esculentin peptide (e.g., stapled Esculentin-1A peptide).

In certain embodiments, the stapled peptide is an inhibitor of a protein-protein interaction (PPI). In certain embodiments, the stapled peptide is an inhibitor of a BCL-2 family member protein (e.g., BCL-x_{L}, BCL-2, BCL-W, or MCL1). In certain embodiments, the stapled peptide is an activator of a BCL-2 family member protein effector (e.g., BAX, BAK, or BOK). In certain embodiments, the stapled peptide is a stapled BCL-2-interacting mediator of cell death (BIM) peptide. In certain embodiments, the stapled peptide is a β-catenin inhibitor (e.g., an inhibitor of Wnt/β-catenin signaling). In certain embodiments, the stapled peptide is an MDM2 and/or MDMX inhibitor (e.g., inhibits the binding of MDM2 and/or MDMX to p53). Other examples of stapled peptides are provided herein.

In certain embodiments, the antibody or antigen-binding fragment thereof is directly conjugated to the stapled peptide (e.g., via a bond). In certain embodiments, the antibody or antigen-binding fragment thereof is conjugated to the stapled peptide via a linker. In certain embodiments, the linker is a cleavable linker (e.g., a pH cleavable linker, or a linker cleavable by a protease, esterase, or intracellular disulfide reduction). In certain embodiments, the linker is a peptidic linker (e.g., a cleavable peptidic linker).

Also provided herein are pharmaceutical compositions comprising a SPAC provided herein and a pharmaceutically acceptable carrier. In certain embodiments, the pharmaceutical composition comprises a therapeutically effective amount of a SPAC provided herein (e.g., for treating cancer or inhibiting tumor growth in a subject).

Stapled-peptide antibody conjugates (SPACs) provided herein can deliver biologically active stapled peptides to cells (e.g., cancer cells, bacterial cells), and are therefore useful in the treatment and/or prevention of various diseases (e.g., proliferative diseases such as cancer, infectious diseases). Methods provided herein include:
(i) Methods of treating and/or preventing a disease in a subject comprising administering to the subject a therapeutically and/or prophylactically effective amount of a SPAC provided herein, or a pharmaceutical composition thereof. In certain embodiments, the disease is a proliferative disease (e.g., cancer);
(ii) Methods of treating a cancer in a subject comprising administering to the subject a therapeutically effective amount of a SPAC provided herein, or a pharmaceutical composition thereof;
(iii) Methods of inhibiting tumor growth in a subject comprising administering to the subject an effective amount of a SPAC provided herein, or a pharmaceutical composition thereof;
(iv) Methods of delivering a stapled peptide into a cell comprising contacting the cell with a SPAC provided herein, or a pharmaceutical composition thereof. In certain embodiments, the cell is a cancer cell. In certain embodiments, the stapled peptide in delivered to the cell in vitro. In certain embodiments, the stapled peptide is delivered to the cell in vivo (i.e., in a subject); and
(v) Method of triggering cancer cell death comprising contacting the cancer cell with an effective amount of a SPAC provided herein, or a pharmaceutical composition thereof. In certain embodiments, the stapled peptide in delivered to the cell in vitro. In certain embodiments, the stapled peptide is delivered to the cell in vivo (i.e., in a subject).
(vi) Methods of treating and/or preventing an infectious disease (e.g., bacterial infection) in a subject comprising administering to the subject an effective amount of a SPAC provided herein, or a pharmaceutical composition thereof,
(vii) Methods of killing and/or inhibiting the growth of bacteria comprising contacting the bacteria with an effective amount of a SPAC provided herein, or a pharmaceutical composition thereof,

Also provided here are SPACs described herein, and pharmaceutical compositions thereof, for use in any of the methods described herein. In another aspect, provided herein are uses of SPACs described herein, and pharmaceutical compositions thereof, for the manufacture of medicaments.

In another aspect, provided herein are kits comprising a SPAC provided herein, or a pharmaceutical composition thereof. The kits described herein may include a single dose or multiple doses of the SPAC or pharmaceutical composition thereof. The kits described herein are useful in any method or use provided herein, and optionally further comprise instructions for using the kit (e.g., instructions for using the SPAC or composition included in the kit).

Also provided herein a methods of preparing a SPAC described herein.

The details of certain embodiments of the invention are set forth in the Detailed Description of Certain Embodiments, as described below. Other features, objects, and advantages of the invention will be apparent from the Definitions, Examples, Figures, and Claims.

### Definitions

### General Definitions

The following definitions are general terms used throughout the present application.

The terms “peptide” and “polypeptide” are used interchangeably and refer to a polymer of amino acid residues linked together by peptide bonds. The terms also include proteins, and refer to peptides, polypeptides, and proteins, of any size, structure, or function. Typically, a peptide will be at least three amino acids long, or at least the length required by an amino acid sequence provided herein. A peptide may refer to an individual peptide or a collection of peptides. Peptides provided herein can include natural amino acids and/or unnatural amino acids (i.e., compounds that do not occur in nature but that can be incorporated into a peptide chain) in any combination. One or more of the amino acids in a peptide may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A peptide may be a fragment or modified version of a naturally occurring peptide or protein. A peptide may be naturally occurring, recombinant, synthetic, or any combination of these.

The term “amino acid” refers to a molecule containing both an amino group and a carboxyl group. Amino acids include alpha-amino acids, the generic structure of which is depicted below. Each amino acid referred to herein may be denoted by a 1- to 4-letter code (e.g., R and Arg represent L-Arginine, hArg represents L-homoarginine).

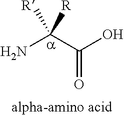

Suitable amino acids include, without limitation, natural alpha-amino acids such as D- and L-isomers of the 20 common naturally occurring alpha-amino acids found in peptides (e.g., A, R, N, C, D, Q, E, G, H, I, L, K, M, F, P, S, T, W, Y, V, as provided below), and unnatural alpha-amino acids.

Exemplary natural alpha-amino acids (with one-letter code provided in parentheses) include L-alanine (A), L-arginine (R), L-asparagine (N), L-aspartic acid (D), L-cysteine (C), L-glutamic acid (E), L-glutamine (Q), glycine (G), L-histidine (H), L-isoleucine (I), L-leucine (L), L-lysine (K), L-methionine (M), L-phenylalanine (F), L-proline (P), L-serine (S), L-threonine (T), L-tryptophan (W), L-tyrosine (Y), and L-valine (V).

Exemplary unnatural alpha-amino acids include D-arginine, D-asparagine, D-aspartic acid, D-cysteine, D-glutamic acid, D-glutamine, D-histidine, D-isoleucine, D-leucine, D-lysine, D-methionine, D-phenylalanine, D-proline, D-serine, D-threonine, D-tryptophan, D-tyrosine, D-valine, Di-vinyl, α-methyl-alanine (Aib), α-methyl-arginine, α-methyl-asparagine, α-methyl-aspartic acid, α-methyl-cysteine, α-methyl-glutamic acid, α-methyl-glutamine, α-methyl-histidine, α-methyl-isoleucine, α-methyl-leucine, α-methyl-lysine, α -methyl-methionine, α-methyl-phenylalanine, α-methyl-proline, α-methyl-serine, α-methyl-threonine, α-methyl-tryptophan, α-methyl-tyrosine, α-methyl-valine, norleucine, and terminally unsaturated alpha-amino acids. There are many known unnatural amino acids any of which may be included in the peptides of the present disclosure. See for example, S. Hunt, The Non-Protein Amino Acids: In Chemistry and Biochemistry of the Amino Acids, edited by G. C. Barrett, Chapman and Hall, 1985. Unnatural amino acids also include amino acids comprising nitrogen substituents.

Certain amino acids referred to herein are provided in Table 1 below (represented by name, structure, and 1- to 4-letter code).

TABLE 1
Certain Amino Acids

| Name | Code | Structure |
|---|---|---|
| L-Norleucine | B | 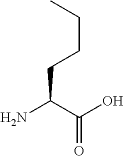 |
| L-Ornithine | Orn | 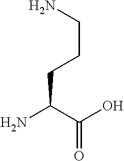 |
| L-Diaminobutyric Acid | Dab | 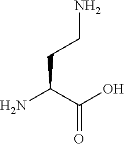 |
| L-Diaminopropionic Acid | Dap | 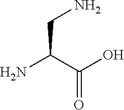 |
| β-Alanine | βAla | 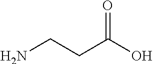 |
| L-Homoarginine | hArg | 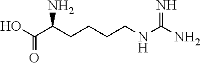 [SMILES: N=C(N)NCCCC[C@H](N)C(=O)O] |
| D-Phenylalanine | F¹ | 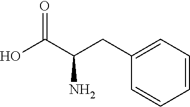 [SMILES: N[C@H](Cc1ccccc1)C(=O)O] |
| L-4-trifluoromethyl phenylalanine | F² | 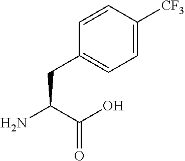 [SMILES: N[C@@H](Cc1ccc(C(F)(F)F)cc1)C(=O)O] |
| L-4-t-butyl phenylalanine | F³ | 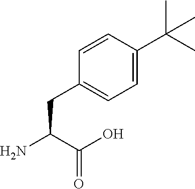 [SMILES: CC(C)(C)c1ccc(C[C@H](N)C(=O)O)cc1] |
| L-4-fluoro phenylalanine | F⁴ | 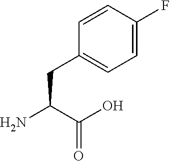 [SMILES: N[C@@H](Cc1ccc(F)cc1)C(=O)O] |
| L-4-methyl phenylalanine | F⁵ | 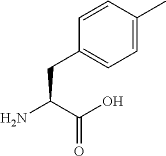 |
| S-2-(4′-pentenyl) alanine | S⁵ | 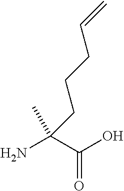 |
| R-2-(7′-octeny1)alanine | R⁸ | 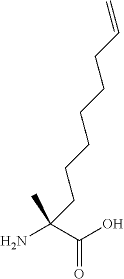 |
| L-propargyl glycine | J | 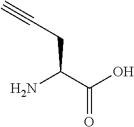 |
| L-Azidolysine | Azi | 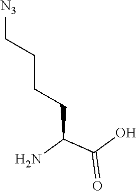 [SMILES: [N-]=[N+]=NCCCC[C@H](N)C(=O)O] |

The term “amino acid substitution” when used in reference to an amino acid sequence refers to an amino acid of the amino acid sequence being replaced by a different amino acid (e.g., replaced by a natural or unnatural amino acid). An amino acid sequence provided herein may comprise or include one or more amino acid substitutions. Specific amino acid substitutions are denoted by commonly used colloquial nomenclature in the art of peptide sequencing to denote amino acid sequence variations. For example, when referring to SEQ ID NO: 2 (below), an “amino acid substitution at H7” refers to the histidine (H) at position 7 of the amino acid sequence being replaced by a different amino acid (e.g., a natural or unnatural amino acid other than histidine). Also, for example, when referring to SEQ ID NO: 2, the amino acid substitution “H7K” refers to replacing the histidine (H) at position 7 of the amino acid sequence of SEQ ID NO: 2 with lysine (K), resulting in an amino acid sequence represented by SEQ ID NO: 3 (below).

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | Position # |
|---|---|---|---|---|---|---|---|---|---|---|---|---|---|---|---|---|---|---|---|---|---|---|---|
| G | X¹ | G | K | F | X² | H | S | K | K | K | F | G | K | A | X³ | V | G | E | X⁴ | A | K | K | SEQ ID NO: 2 |
| G | X¹ | G | K | F | X² | K | S | K | K | K | F | G | K | A | X³ | V | G | E | X⁴ | A | K | K | SEQ ID NO: 3 |

The term “amino acid addition” when used in reference to an amino acid sequence refers to an amino acid (e.g., a natural or unnatural amino acid) being inserted between two amino acids of the amino acid sequence, or added at either end of the sequence. Standard colloquial nomenclature is used to represent specific amino additions (e.g., when referring to SEQ ID NO: 2, “G3_K4insX” denotes that a hypothetical amino acid X is inserted between amino acids G3 and K4 of the amino acid sequence). In certain embodiments, an amino acid sequence herein can comprise 0, 1, 2, 3, 4, 5, 6, 7, 8, 9, or 10 amino acid additions.

The term “amino acid deletion” when used in reference to an amino acid sequence refers to an amino acid of the amino acid sequence being deleted from the amino acid sequence. Standard colloquial nomenclature is used to represent specific amino deletions (e.g., when referring to SEQ ID NO: 2, “G1del” denotes that the amino acid G1 is deleted from the sequence). In certain embodiments, an amino acid sequence herein can comprise 0, 1, 2, 3, 4, 5, 6, 7, 8, 9, or 10 amino acid deletions.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the peptides of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the peptides of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Throughout the present disclosure, references to a “stapled peptide” are intended to encompass peptides comprising any amino acid sequence provided herein (including any disclosed amino acid substitutions, additions, deletions, and/or modifications), and pharmaceutically acceptable salts, stereoisomers, tautomers, isotopically labeled derivatives, solvates, hydrates, polymorphs, co-crystals, and prodrugs thereof.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a peptide described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a SPAC described herein refers to an amount sufficient to elicit the desired biological response. An effective amount of a SPAC described herein may vary depending on such factors as the desired biological endpoint, severity of side effects, disease, or disorder, the identity, pharmacokinetics, and pharmacodynamics of the particular peptide, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a SPAC described herein in a single dose. In certain embodiments, an effective amount is the combined amounts of a SPAC described herein in multiple doses.

A “therapeutically effective amount” of a SPAC described herein is an amount sufficient to provide a therapeutic benefit in the treatment of a condition or to delay or minimize one or more symptoms associated with the condition. A therapeutically effective amount of a SPAC means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a SPAC described herein is an amount sufficient to prevent a condition, or one or more symptoms associated with the condition or prevent its recurrence. A prophylactically effective amount of a SPAC means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Peptides described herein can comprise one or more asymmetric centers, and thus can exist in various stereoisomeric forms, e.g., enantiomers and/or diastereomers. For example, the peptides described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses peptides as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond. Additionally, the bond [symbol] or [symbol] is a double or triple bond.

Unless otherwise provided, formulae and structures depicted herein include peptides that do not include isotopically enriched atoms, and also include peptides that include isotopically enriched atoms (“isotopically labeled derivatives”). For example, peptides having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such peptides are useful, for example, as analytical tools or probes in biological assays. The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 6 carbon atoms (“C₁₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like.

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”).

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). The one or more carbon-carbon double bonds can be internal (such as in 2-butenyl) or terminal (such as in 1-butenyl). In an alkenyl group, a C═C double bond for which the stereochemistry is not specified (e.g., —CH═CHCH₃ or

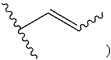

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”).

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). The one or more carbon-carbon triple bonds can be internal (such as in 2-butynyl) or terminal (such as in 1-butynyl).

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”).

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 6 ring carbon atoms (“C₃₋₆ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur. In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom or the ring that does not contain a heteroatom.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A chemical moiety is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, heteroaryl, acyl groups are optionally substituted. In general, the term “substituted” when referring to a chemical group means that at least one hydrogen present on the group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The invention is not limited in any manner by the exemplary substituents described herein.

Exemplary substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SCN, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})Ra, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —R^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂—NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl; wherein X⁻ is a counterion;
or two geminal hydrogens on a carbon atom are replaced with the group ═O, ═S, ═NN(R^{bb})₂, —NNR^{bb}C(═O)R^{aa}, —NNR^{bb}C(═O)OR^{aa}, —NNR^{bb}S(═O)₂R^{aa}, =NR^{bb}, or =NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring; and each X⁻ is a counterion.

In certain embodiments, each substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —N₃, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{cc})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S—SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino.

In certain embodiments, the “substituted amino” is a monosubstituted amino or a disubstituted amino group. The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen. The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen. The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

These and other exemplary substituents are described in more detail in the Detailed Description, Examples, Figures, and Claims. The invention is not limited in any manner by the above exemplary listing of substituents.

### BRIEF DESCRIPTION OF THE DRAWINGS

The accompanying drawings, which are incorporated in and constitute a part of this specification, illustrate several embodiments of the invention and together with the description, provide non-limiting examples of the invention.

FIGS. 1A-1B show cytotoxicity of anti-HER2 SPACs in breast cancer cell lines. FIG. 1A shows cytotoxicity of anti-HER2 SPACs (SPAC 1 and SPAC 2) in BT-474 cell line with high HER2 expression (HER2+++). FIG. 1B shows cytotoxicity of anti-HER2 SPACs (SPAC 1 and SPAC 2) in MCF7 cell line with low HER2 expression (HER2+).

FIG. 2 shows cytotoxicity of an anti-CD38 SPAC (SPAC 3) in a CD38+ multiple myeloma cell line, RPMI 8226.

FIG. 3 shows inhibition of cellular proliferation with an anti-CD38 SPAC (SPAC 11) comprising a stapled MCL-1 inhibitor peptide in two CD38+ multiple myeloma cell lines (NCI-H929 and RPMI 8226).

FIG. 4 shows inhibition of cellular proliferation with an anti-CD38 SPAC (SPAC 8) comprising a stapled MDM2 inhibitor peptide in a CD38+ multiple myeloma cell line (RPMI 8226).

FIG. 5 shows inhibition of cellular proliferation with an anti-CD38 SPAC (SPAC 16) comprising a stapled β-catenin inhibitor peptide in a CD38+ multiple myeloma cell line (RPMI 8226).

FIGS. 6A-6B show activity of an anti-HER2 SPAC (SPAC 7) comprising a stapled MDM2 inhibitor peptide compared to a traditional ADC (trastuzumab emtansine) in a HER2-low p53 WT breast cancer cell line (MCF7) (FIG. 6A) and a HER2+++p53 mut breast cancer cell line (SK-BR-3) (FIG. 6B).

FIG. 7A shows cytotoxic activity of a stapled peptide comprising SEQ ID NO: 73 in breast cancer cell lines. FIG. 7B shows cytotoxic activity of a stapled peptide comprising SEQ ID NO: 24 in breast cancer cell lines.

### DETAILED DESCRIPTION OF CERTAIN EMBODIMENTS

Provided herein are stapled peptide-antibody conjugates (SPACs) comprising a stapled peptide conjugated to an antibody or antigen-binding fragment thereof. In certain embodiments, the stapled peptide is conjugated to the antibody or antigen-binding fragment thereof via a linker. Also provided herein are pharmaceutical compositions and kits comprising SPACs provided herein, methods of preparing SPACs provided herein, as well as methods of using the SPACs provided herein (e.g., for treating a disease (e.g., cancer) in a subject, delivering a stapled peptide to the cell (e.g., cancer cell) of a subject, etc.).

### Stapled Peptide-Antibody Conjugates (SPACs)

The present disclosure provides stapled peptide-antibody conjugates (SPACs) comprising a stapled peptide conjugated to an antibody or antigen-binding fragment thereof. In certain embodiments, the stapled peptide is conjugated to the antibody or antigen-binging fragment thereof via a linker (e.g., a cleavable linker). In certain embodiments, the antibody or antigen-binding fragment thereof is an anti-cancer antibody or antigen binding fragment thereof; and the stapled peptide is a stapled anti-cancer peptide.

### Antibodies and Antigen-Binding Fragments

The stapled peptide-antibody conjugates (SPACs) provided herein comprise an antibody or antigen-binding fragment thereof. As used herein, the term “antibody” refers to a molecule that specifically binds to, or is immunologically reactive with, a particular antigen and includes at least the variable domain of a heavy chain, and normally includes at least the variable domains of a heavy chain and of a light chain of an immunoglobulin. Unless otherwise indicated, the term “antibody” (Ab) is meant to include both intact (whole) molecules as well as antibody fragments (e.g., Fab and F(ab′)₂ fragments) that are capable of specifically binding to a target antigen. Antibodies (including intact antibodies and antigen-binding fragments), variants, and derivatives thereof include, but are not limited to, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments (e.g., Fab, Fab′, and F(ab′)₂, Fd, Fvs, single-chain Fvs (scFv), rlgG, single-chain antibodies, disulfide-linked Fvs (sdFv), fragments containing either a V_{L} or V_{H} domain, fragments produced by an Fab expression library), and anti-idiotypic (anti-Id) antibodies. Fab and F(ab′)₂ fragments, for example, lack the Fc fragment of an intact antibody. Antibody molecules of the conjugates can be of any type (e.g., IgG, IgE, IgM, IgD, IgA, and IgY), class (e.g., IgG1, IgG2, IgG3, IgG4, IgA1 and IgA2) or subclass of immunoglobulin molecule.

The term “antigen-binding fragment,” as used herein, refers to one or more fragments of an immunoglobulin that retain the ability to specifically bind to a target antigen. The antigen-binding function of an immunoglobulin can be performed by fragments of a full-length antibody. The antibody fragments can be, e.g., a Fab, F(ab′)₂, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment containing two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb (Ward et al., Nature, 1989, 341, 544-546) including V_{H} and V_{L} domains; (vi) a dAb fragment that consists of a V_{H} domain; (vii) a dAb that consists of a V_{H} or a V_{L} domain; (viii) an isolated complementarity determining region (CDR); and (ix) a combination of two or more isolated CDRs which may optionally be joined by a linker, e.g., a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv)). These antibody fragments (i.e., “antigen-binding fragments”) can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

Antibodies described herein can be murine, rat, human, or of any other origin (including chimeric or humanized antibodies and fragments thereof). Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In certain embodiments, the antibody is a monoclonal antibody (mAb) or antigen-binding fragment thereof. In certain embodiments the antibody is an intact (i.e., whole) mAb. In certain embodiments, the antibody is an antigen-binding fragment of a mAb. Examples of monoclonal antibodies (mAbs) (including generic name, trade name(s), known target antigen(s), and exemplary use(s)) are provided below in Table 2. Therapeutic applications of the mAbs listed below are not limited the particular known target antigens and exemplary uses provided.

TABLE 2
Examples of Monoclonal Antibodies (mAbs)

| | | Known Target | |
|---|---|---|---|
| Generic Name | Trade Name(s) | Antigen(s) | Exemplary Use(s) |
| 3F8 | | GD2 ganglioside | neuroblastoma |
| Abagovomab | | CA-125 | ovarian cancer |
| Abciximab | ReoPro | CD41 (integrin alpha-IIb) | platelet aggregation inhibitor |
| Abituzumab | | CD51 | cancer |
| Abrezekimab | | interleukin 13 | |
| Abrilumab | | integrin α₄ β₇ | inflammatory bowel |
| | | | disease, ulcerative colitis, Crohn's |
| | | | disease |
| Actoxumab | | Clostridium difficile | Clostridium difficile colitis |
| Adalimumab | Humira | TNF-α | rheumatoid arthritis, Crohn's |
| | | | disease, plaque psoriasis, psoriatic |
| | | | arthritis, ankylosing |
| | | | spondylitis, juvenile idiopathic |
| | | | arthritis, hemolytic disease of the |
| | | | newborn |
| Adecatumumab | | EpCAM | prostate and breast cancer |
| Aducanumab | Aduhelm | beta-amyloid | Alzheimer's disease |
| Afasevikumab | | IL17A and IL17F | multiple sclerosis |
| Afelimomab | | TNF-α | sepsis |
| Alacizumab | | VEGFR2 | cancer |
| Alemtuzumab | Lemtrada, Campath | CD52 | multiple sclerosis |
| Alirocumab | Praluent | PCSK9 | hypercholesterolemia |
| Altumomab | Hybri-ceaker | Carcinoembryonic | colorectal cancer |
| | (Altumomab | antigen (CEA) | |
| | pentetate) | | |
| Amatuximab | | mesothelin | cancer |
| Amivantamab | Rybrevant | Epidermal growth factor | non-small cell lung cancer |
| | | receptor (EGFR), cMet | (NSCLC) |
| Anatumomab | | Tumor-associated | non-small cell lung cancer |
| | | glycoprotein 72 (TAG-72) | |
| Andecaliximab | | gelatinase B | gastric cancer, gastroesophageal |
| | | | cancer |
| Anetumab | | mesothelin (MSLN) | cancer |
| Anifrolumab | Saphnelo | interferon α/β receptor | systemic lupus erythematosus |
| Ansuvimab | Ebanga | Ebola virus glycoprotein | Ebola virus |
| Anrukinzumab | | IL-13 | asthma |
| Apolizumab | | HLA-DR | hematological cancers |
| Aprutumab | | FGFR2 | |
| Arcitumomab | CEA-Scan | Carcinoembryonic | gastrointestinal cancers |
| | | antigen (CEA) | |
| Ascrinvacumab | | activin receptor-like | cancer |
| | | kinase 1 | |
| Aselizumab | | L-selectin (CD62L) | severely injured patients |
| Atezolizumab | Tecentriq | PD-L1 | bladder, non-small cell lung, and |
| | | | triple-negative breast cancers |
| Atidortoxumab | | Staphylococcus | |
| | | aureus alpha toxin | |
| Atinumab | | RTN4 | |
| Atoltivimab | | | Zaire ebolavirus (Ebola virus) |
| Atorolimumab | | Rhesus factor | hemolytic disease of the newborn |
| Avelumab | Bavencio | PD-L1 | urothelial carcinoma and Merkel |
| | | | cell carcinoma |
| Azintuxizumab | | CD319 | cancer |
| Bamlanivimab | | spike protein receptor | coronavirus disease 2019 (COVID- |
| | | binding domain (RBD) of | 19) |
| | | SARS-CoV-2 | |
| Bapineuzumab | | beta amyloid | Alzheimer's disease |
| Basiliximab | Simulect | CD25 (α chain of IL-2 | prevention of organ transplant |
| | | receptor) | rejections |
| Bavituximab | | phosphatidylserine | cancer, viral infections |
| BCD-100 | | PD-1 | melanoma |
| Bectumomab | LymphoScan | CD22 | non-Hodgkin's |
| | | | lymphoma (detection) |
| Begelomab | | DPP4 | |
| Belantamab | Blenrep | B-cell maturation | multiple myeloma |
| | (Belantamab | antigen (BCMA) | |
| | mafodotin) | | |
| Belimumab | Benlysta | B-cell activating | systemic lupus erythematosus |
| | | factor (BAFF) | without renal or CNS involvement |
| Bemarituzumab | | FGFR2 | gastric cancer or gastroesophageal |
| | | | junction adenocarcinoma |
| Benralizumab | Fasenra | CD125 | asthma |
| Berlimatoxumab | | Staphylococcus aureus bi- | |
| | | component leukocidin | |
| Bermekimab | Xilonix | IL1A | colorectal cancer |
| Bersanlimab | | ICAM-1 | |
| Bertilimumab | | CCL11 (eotaxin-1) | severe allergic disorders |
| Besilesomab | Scintimun | Carcinoembryonic | inflammatory lesions and |
| | | antigen (CEA)-related | metastases (detection) |
| | | antigen | |
| Bevacizumab | Avastin | VEGF | colorectal, non-small cell lung, |
| | | | renal, glioblastoma, and ovarian |
| | | | cancers |
| Bezlotoxumab | Zinplava | Clostridium difficile | Clostridium difficile colitis |
| Biciromab | FibriScint | fibrin II, beta chain | thromboembolism (diagnosis) |
| Bimagrumab | | ACVR2B | myostatin inhibitor |
| Bimekizumab | Bimzelx | IL17A, IL17F, IL17AF | psoriasis |
| Birtamimab | | serum amyloid A protein | amyloidosis |
| Bivatuzumab | | CD44 v6 | squamous cell carcinoma |
| Bleselumab | | CD40 | organ transplant rejection |
| Blinatumomab | Blincyto | CD19 | pre-B Acute lymphoblastic |
| | | | leukemia (ALL) (CD19+) |
| Blontuvetmab | Blontress | CD20 | |
| Blosozumab | | SOST | osteoporosis |
| Bococizumab | | PCSK9 | dyslipidemia |
| Brazikumab | | IL23 | Crohn's disease |
| Brentuximab | Adcentris | CD30 (TNFRSF8) | Hodgkin's lymphoma, |
| | (Brentuximab | | anaplastic large-cell lymphoma |
| | vedotin) | | |
| Briakinumab | | IL-12, IL-23 | psoriasis, rheumatoid |
| | | | arthritis, inflammatory bowel |
| | | | diseases, multiple sclerosis |
| Brodalumab | Siliq | IL-17 | plaque psoriasis |
| Brolucizumab | Beovu | vascular endothelial | wet age-related macular |
| | | growth factor A (VEGFA) | degeneration |
| Brontictuzumab | | Notch 1 | cancer |
| Burosumab | Crysvita | FGF 23 | X-linked hypophosphatemia |
| Cabiralizumab | | CSF1R | metastatic pancreatic cancer |
| Camidanlumab | | CD25 (α chain of IL-2 | B-cell Hodgkin's lymphoma, non- |
| | | receptor) | Hodgkin lymphoma, acute |
| | | | lymphoblastic leukemia, acute |
| | | | myeloid leukemia |
| Camrelizumab | | PD-1 | hepatocellular carcinoma |
| Canakinumab | Ilaris | IL-1 | cryopyrin-associated periodic |
| | | | syndrome |
| Cantuzumab | | CanAg (a glycoform | cancer |
| | | of MUC1) | |
| Caplacizumab | Cablivi | VWF | thrombotic thrombocytopeni |
| | | | purpura, thrombosis |
| Casirivimab | | spike protein receptor | coronavirus disease 2019 (COVID- |
| | | binding domain (RBD) of | 19) |
| | | SARS-CoV-2 | |
| Capromab | Prostascint | Glutamate | prostate cancer |
| | | carboxypeptidase II | |
| Carlumab | | MCP-1 | oncology/immune indications |
| Carotuximab | | endoglin | angiosarcoma |
| Catumaxomab | Removab | EpCAM, CD3 | ovarian cancer, malignant ascites, |
| | | | gastric cancer |
| CBR96 | | Lewis-Y antigen | cancer |
| Cedelizumab | | CD4 | prevention of organ transplant |
| | | | rejections, treatment of |
| | | | autoimmune diseases |
| Cemiplimab | Libtayo | PD-1 | cutaneous squamous cell carcinoma |
| Cergutuzumab | | IL2 | cancer |
| Certolizumab | Cimzia | TNF-α | Crohn's disease, rheumatoid |
| | (Certolizumab | | arthritis, axial |
| | pegol) | | spondyloarthritis, psoriasis arthritis |
| Cetrelimab | | PD-1 | cancer |
| Cetuximab | Erbitux | Epidermal growth factor | colorectal cancer and head and |
| | | receptor (EGFR) | neck squamous cell carcinoma |
| Cibisatamab | | CEACAM5 | cancer |
| Cirmtuzumab | | ROR1 | chronic lymphocytic leukemia |
| Citatuzumab | | EpCAM | ovarian cancer and other solid |
| | | | tumors |
| Cixutumumab | | IGF-1 receptor (CD221) | solid tumors |
| Clazakizumab | | Interleukin 6 (IL-6) | rheumatoid arthritis |
| Clenoliximab | | CD4 | rheumatoid arthritis |
| Clivatuzumab | hPAM4-Cide | MUC1 | pancreatic cancer |
| | (Clivatuzumab | | |
| | tetraxetan) | | |
| Codrituzumab | | glypican 3 | cancer |
| Cofetuzumab | | PTK7 | cancer |
| Coltuximab | | CD19 | cancer |
| Conatumumab | | TRAIL-R2 | cancer |
| Concizumab | | tissue factor pathway | bleeding |
| | | inhibitor (TFPI) | |
| Cosfroviximab | ZMapp | ebolavirus glycoprotein | Ebola virus |
| Crenezumab | | 1-40-β-amyloid | Alzheimer's disease |
| Crizanlizumab | Adakveo | P-selectin | sickle-cell disease |
| Crotedumab | | glucagon | diabetes |
| | | receptor (GCGR) | |
| CR6261 | | Influenza A | infectious disease/influenza A |
| | | hemagglutinin | |
| Cusatuzumab | | CD70 | cancer |
| Dacetuzumab | | CD40 | hematologic cancers |
| Daclizumab | Zenapax | CD25 (α chain of IL-2 | prevention of organ transplant |
| | | receptor) | rejections, multiple sclerosis |
| Dalotuzumab | | IGF-1 receptor (CD221) | cancer |
| Dapirolizumab | | CD154 (CD40L) | |
| Daratumumab | Darzalex | CD38 | multiple myeloma |
| Dectrekumab | | IL-13 | |
| Demcizumab | | DLL4 | cancer |
| Denintuzumab | | CD19 | cancer |
| Denosumab | Prolia | RANKL | osteoporosis, bone metastases |
| Depatuxizumab | | EGFR | glioblastoma |
| Derlotuximab | | histone complex | recurrent glioblastoma multiforme |
| Detumomab | | B-lymphoma cell | lymphoma |
| Dezamizumab | | serum amyloid P | |
| | | component | |
| Dinutuximab | Unituxin | GD2 ganglioside | neuroblastoma |
| Dinutuximab | Qarziba | GD2 ganglioside | neuroblastoma |
| beta | | | |
| Diridavumab | | hemagglutinin | influenza A |
| Domagrozumab | | GDF-8 | Duchenne muscular dystrophy |
| Dorlimomab | | | |
| Dostarlimab | Jemperli | PD-1 | endometrial cancer |
| Drozitumab | | DR5 | cancer |
| DS-8201 | | HER2 | gastric or gastroesophageal |
| | | | junction adenocarcinoma |
| Duligotuzumab | | ERBB3 (HER3) | testicular cancer |
| Dupilumab | Dupixent | IL-4Rα | atopic dermatitis, asthma, nasal |
| | | | polyps |
| Durvalumab | Imfinzi | PD-L1 | bladder cancer |
| Dusigitumab | | ILGF2 | B-cell malignancies |
| Duvortuxizumab | | CD19, CD3E | cancer |
| Ecromeximab | | GD3 ganglioside | malignant melanoma |
| Eculizumab | Soliris | C5 | paroxysmal nocturnal |
| | | | hemoglobinuria, atypical hemolytic |
| | | | uremic syndrome |
| Edobacomab | | endotoxin | sepsis caused by Gram- |
| | | | negative bacteria |
| Edrecolomab | Panorex | EpCAM | colorectal carcinoma |
| Efalizumab | Raptiva | LFA-1 (CD11a) | psoriasis |
| Efungumab | Mycograb | Hsp90 | invasive Candida infection |
| Eldelumab | | interferon gamma-induced | Crohn's disease, ulcerative colitis |
| | | protein | |
| Elezanumab | | repulsive guidance | spinal cord injury and multiple |
| | | molecule A (RGMA) | sclerosis |
| Elgemtumab | | ERBB3 (HER3) | cancer |
| Elotuzumab | Empliciti | SLAMF7 | multiple myeloma |
| Elsilimomab | | IL-6 | |
| Emactuzumab | | CSF1R | cancer |
| Emapalumab | Gamifant | interferon gamma | hemophagocytic |
| | | | lymphohistiocytosis |
| Emibetuzumab | | hHGFR | cancer |
| Emicizumab | Hemlibra | activated F9, F10 | hemophilia A |
| Enapotamab | | AXL | cancer |
| Enavatuzumab | | TWEAK receptor | cancer |
| Enfortumab | Padcev | nectin-4 | bladder cancer |
| | (Enfortumab | | |
| | vedotin) | | |
| Enlimomab | | ICAM-1 (CD54) | |
| Enoblituzumab | | CD276 | cancer |
| Enokizumab | | IL9 | asthma |
| Enoticumab | | DLL4 | |
| Ensituximab | | MUC5AC | cancer |
| Epcoritamab | | CD3, CD20 | B-cell lymphoma |
| Epitumomab | | episialin | |
| Epratuzumab | | CD22 | cancer, systemic lupus |
| | | | erythematosus (SLE) |
| Eptinezumab | Vyepti | calcitonin gene-related | migraine |
| | | peptide | |
| Erenumab | Aimovig | calcitonin gene-related | migraine |
| | | peptide receptor (CGRP) | |
| Erlizumab | | ITGB2 (CD18) | heart attack, stroke, traumatic |
| | | | shock |
| Ertumaxomab | Rexomun | HER2/neu, CD3 | breast cancer |
| Etaracizumab | Abegrin | integrin αᵥβ₃ | melanoma, prostate cancer, ovarian |
| | | | cancer etc. |
| Etesevimab | | spike protein receptor | coronavirus disease 2019 (COVID- |
| | | binding domain (RBD) of | 19) |
| | | SARS-CoV-2 | |
| Etigilimab | | TIGIT | |
| Etrolizumab | | integrin β₇ | inflammatory bowel disease |
| Evinacumab | Evkeeza | angiopoietin 3 | dyslipidemia |
| Evolocumab | Repatha | PCSK9 | hypercholesterolemia |
| Exbivirumab | | hepatitis B surface antigen | hepatitis B |
| Fanolesomab | NeutroSpec | CD15 | appendicitis |
| Faralimomab | | interferon receptor | |
| Faricimab | | VEGF-A and Ang-2 | angiogenesis, ocular vascular |
| | | | diseases |
| Farletuzumab | | folate receptor 1 | ovarian cancer |
| Fasinumab | | Nerve growth | acute sciatic pain |
| | | factor (HNGF) | |
| FBTA05 | Lymphomun | CD20 | chronic lymphocytic leukemia |
| Felvizumab | | respiratory syncytial virus | respiratory syncytial virus infection |
| Fezakinumab | | IL-22 | rheumatoid arthritis, psoriasis |
| Fibatuzumab | | ephrin receptor A3 | |
| Ficlatuzumab | | Hepatocyte growth | cancer |
| | | factor (HGF) | |
| Figitumumab | | IGF-1 receptor (CD221) | adrenocortical carcinoma, non- |
| | | | small cell lung carcinoma etc. |
| Firivumab | | influenza A virus | |
| | | hemagglutinin | |
| Flanvotumab | | TYRP1 (glycoprotein 75) | melanoma |
| Fletikumab | | IL 20 | rheumatoid arthritis |
| Flotetuzumab | | IL 3 receptor | hematological malignancies |
| Fontolizumab | HuZAF | IFN-γ | Crohn's disease |
| Foralumab | | CD3 epsilon | |
| Foravirumab | | rabies virus glycoprotein | rabies (prophylaxis) |
| Fremanezumab | Ajovy | calcitonin gene-related | migraine |
| | | peptide alpha and beta | |
| Fresolimumab | | TGF-β | idiopathic pulmonary fibrosis, focal |
| | | | segmental glomerulosclerosis, |
| | | | cancer |
| Frovocimab | | PCSK9 | hypercholesterolemia |
| Frunevetmab | | nerve growth | |
| | | factor (NGF) | |
| Fulranumab | | Nerve growth | pain |
| | | factor (NGF) | |
| Futuximab | | Epidermal growth factor | cancer |
| | | receptor (EGFR) | |
| Galcanezumab | Emgality | calcitonin | migraine |
| Galiximab | | CD80 | B-cell lymphoma |
| Gancotamab | | HER2/neu | cancer |
| Ganitumab | | IGF-1 receptor (CD221) | cancer |
| Gantenerumab | | beta amyloid | Alzheimer's disease |
| Gatipotuzumab | | MUC1 | cancer |
| Gavilimomab | | CD147 (basigin) | graft versus host disease |
| Gedivumab | | hemagglutinin HA | |
| Gemtuzumab | Mylotarg | CD33 | acute myelogenous leukemia |
| | (Gemtuzumab | | |
| | ozogamicin) | | |
| Gevokizumab | | IL-1β | diabetes |
| Gilvetmab | | PCDC1 | |
| Gimsilumab | | CSF2 | rheumatoid arthritis |
| Girentuximab | Rencarex | carbonic anhydrase | clear cell renal cell carcinoma |
| | | 9 (CA-IX) | |
| Glembatumumab | | GPNMB | melanoma, breast cancer |
| Golimumab | Simponi | TNF-α | rheumatoid arthritis, psoriatic |
| | | | arthritis, ankylosing spondylitis |
| Gomiliximab | | CD23 (IgE receptor) | allergic asthma |
| Gosuranemab | | tau protein | progressive supranuclear palsy |
| Guselkumab | Tremfya | IL23 | psoriasis |
| Ianalumab | | BAFF-R | autoimmune hepatitis |
| Ibalizumab | Trogarzo | CD4 | HIV infection |
| Sintilimab | | PD-1 | squamous cell non-small cell lung |
| | | | cancer |
| Ibritumomab | Zevalin | CD20 | non-Hodgkin's lymphoma |
| | (Ibritumomab | | |
| | tiuxetan) | | |
| Icrucumab | | VEGFR-1 | cancer etc. |
| Idarucizumab | Praxbind | dabigatran | reversal of anticoagulant effects of |
| | | | dabigatran |
| Ifabotuzumab | | EPHA3 | glioblastoma multiforme |
| Igovomab | Indimacis-125 | CA-125 | ovarian cancer |
| Iladatuzumab | | CD79B | cancer |
| Imalumab | | macrophage migration | cancer |
| | | inhibitory factor (MIF) | |
| Imaprelimab | | melanoma cell adhesion | |
| | | molecule (MCAM) | |
| Imciromab | Myoscint | cardiac myosin | cardiac imaging |
| Imdevimab | | spike protein receptor | coronavirus disease 2019 (COVID- |
| | | binding domain (RBD) of | 19) |
| | | SARS-CoV-2 | |
| Imgatuzumab | | Epidermal growth factor | cancer |
| | | receptor (EGFR) | |
| Inclacumab | | selectin P | cardiovascular disease |
| Indatuximab | | SDC1 | cancer |
| Indusatumab | | GUCY2C | cancer |
| Inebilizumab | Uplizna | CD19 | cancer, systemic sclerosis, multiple |
| | | | sclerosis |
| Infliximab | Remicade | TNF-α | rheumatoid arthritis, ankylosing |
| | | | spondylitis, psoriatic arthritis, |
| | | | psoriasis, Crohn's disease, |
| | | | ulcerative colitis |
| Intetumumab | | CD51 | solid tumors (prostate cancer, |
| | | | melanoma) |
| Inolimomab | | CD25 (α chain of IL-2 | graft versus host disease |
| | | receptor) | |
| Inotuzumab | Besponsa | CD22 | acute lymphoblastic |
| | (Inotuzumab | | leukemia (ALL) |
| | ozogamicin) | | |
| Ipilimumab | Yervoy | CD152, CTLA-4 | melanoma and renal cell carcinoma |
| Iomab-B | | CD45 | ablation of bone marrow |
| Iratumumab | | CD30 (TNFRSF8) | Hodgkin's lymphoma |
| Isatuximab | Sarclisa | CD38 | multiple myeloma |
| Iscalimab | | CD40 | |
| Istiratumab | | IGF1R, CD221 | advanced solid tumors |
| Itolizumab | Alzumab | CD6 | psoriasis |
| Ixekizumab | Taltz | IL 17A | autoimmune diseases |
| Keliximab | | CD4 | chronic asthma |
| Labetuzumab | CEA-Cide | Carcinoembryonic | colorectal cancer |
| | | antigen (CEA) | |
| Lacnotuzumab | | CSF1, macrophage colony | cancer |
| | | stimulating factor (MCSF) | |
| Ladiratuzumab | | LIV-1 | cancer |
| Lampalizumab | | Complement factor | geographic atrophy secondary to |
| | | D (CFD) | age-related macular degeneration |
| Lanadelumab | Takhzyro | kallikrein | angioedema |
| Landogrozumab | | GDF-8 | muscle wasting disorders |
| Laprituximab | | epidermal growth factor | |
| | | receptor (EGFR) | |
| Larcaviximab | | ebolavirus glycoprotein | Ebola virus |
| Lebrikizumab | | IL-13 | asthma |
| Lemalesomab | | NCA-90 (granulocyte | diagnostic agent |
| | | antigen) | |
| Lendalizumab | | C5 | |
| Lenvervimab | | hepatitis B surfage | hepatitis B |
| | | antigen | |
| Lenzilumab | | CSF2 | chronic myelomonocytic |
| | | | leukemia and juvenile |
| | | | myelomonocytic leukemia |
| Lerdelimumab | | TGF beta 2 | reduction of scarring |
| | | | after glaucoma surgery |
| Leronlimab | | CCR5 | breast cancer, HIV |
| Lesofavumab | | hemagglutinin HA | |
| Letolizumab | | tumor necrosis factor | inflammatory diseases |
| | | related activation | |
| | | protein (TRAP) | |
| Lexatumumab | | TRAIL-R2 | cancer |
| Libivirumab | | hepatitis B surface antigen | hepatitis B |
| Lifastuzumab | | phosphate-sodium co- | cancer |
| | | transporter | |
| Ligelizumab | | IGHE | severe asthma and chronic |
| | | | spontaneous urticaria |
| Loncastuximab | Zynlonta | CD19 | diffuse large B-cell lymphoma |
| | (Loncastuximab | | |
| | tesirine) | | |
| Losatuxizumab | | epidermal growth receptor | cancer |
| | | factor (EGRF), ERBB1 H | |
| | | ER1 | |
| Lilotomab | | CD37 | cancer |
| Lintuzumab | | CD33 | cancer |
| Lirilumab | | KIR2D | solid and hematological cancers |
| Lodelcizumab | | PCSK9 | hypercholesterolemia |
| Lokivetmab | Cytopoint | Canis lupus | clinical signs of atopic dermatitis in |
| | | familiaris IL31 | dogs |
| Lorvotuzumab | | CD56 | cancer |
| Lucatumumab | | CD40 | multiple myeloma, non-Hodgkin's |
| | | | lymphoma, Hodgkin's lymphoma |
| Lulizumab | | CD28 | autoimmune diseases |
| Lumiliximab | | CD23 (IgE receptor) | chronic lymphocytic leukemia |
| Lumretuzumab | | ERBB3 (HER3) | cancer |
| Lupartumab | | | |
| Lupartumab | | LYPD3 | |
| Lutikizumab | | interleukin 1 alpha | |
| Maftivimab | | | Zaire ebolavirus (Ebola virus) |
| Mapatumumab | | TRAIL-R1 | cancer |
| Margetuximab | Margenza | HER2 | breast cancer |
| Marstacimab | | tissue factor pathway | bleeding with hemophilia |
| | | inhibitor (TFPI) | |
| Maslimomab | | T-cell receptor | |
| Mavrilimumab | | GMCSF receptor α-chain | rheumatoid arthritis |
| Matuzumab | | Epidermal growth factor | colorectal, lung and stomach cancer |
| | | receptor (EGFR) | |
| Mepolizumab | Bosatria | IL-5 | asthma and white blood cell |
| | | | diseases |
| Metelimumab | | TGF beta 1 | systemic scleroderma |
| Milatuzumab | | CD74 | multiple myeloma and other |
| | | | hematological malignancies |
| Minretumomab | | TAG-72 | tumor detection and therapy |
| Mirikizumab | | IL23A | psoriasis |
| Mirvetuximab | | folate receptor alpha | ovarian cancer |
| soravtansine | | | |
| Mitumomab | | GD3 ganglioside | small cell lung carcinoma |
| Modotuximab | | EGFR extracellular | cancer |
| | | domain III | |
| Mogamulizumab | Poteligeo | CCR4 | cutaneous T-cell lymphoma |
| Monalizumab | | NKG2A | rheumatoid arthritis, gynecologic |
| | | | malignancies, and other cancers |
| Morolimumab | | Rhesus factor | |
| Mosunetuzumab | | CD3E, MS4A1, CD20 | cancer |
| Motavizumab | Numax | respiratory syncytial virus | respiratory syncytial virus |
| | | | (prevention) |
| Moxetumomab | Lumoxiti | CD22 | hairy cell leukemia |
| | (Moxetumomab | | |
| | pasudotox) | | |
| Muromonab- | Orthoclone OKT3 | CD3 | prevention of organ transplant |
| CD3 | | | rejections |
| Nacolomab | | C242 antigen | colorectal cancer |
| Namilumab | | CSF2 | |
| Naptumomab | | 5T4 | non-small cell lung |
| | | | carcinoma, renal cell carcinoma |
| Naratuximab | | CD37 | |
| Narnatumab | | MST1R (aka RON) | cancer |
| Natalizumab | Tysabri | integrin α₄ | multiple sclerosis, Crohn's disease |
| Navicixizumab | | DLL4 and VEGFA | cancer |
| Navivumab | | influenza A | |
| | | virus hemagglutinin HA | |
| Naxitamab | Danyelza | c-Met, GD2 | neuroblastoma |
| Nebacumab | | endotoxin | sepsis |
| Necitumumab | Portrazza | Epidermal growth factor | Non-small cell lung cancer |
| | | receptor (EGFR) | |
| Nemolizumab | | IL31RA | eczema |
| NEOD001 | | amyloid | primary systemic amyloidosis |
| Nerelimomab | | TNF-α | |
| Nesvacumab | | angiopoietin 2 | cancer |
| Netakimab | Efleira | interleukin 17A | plaque psoriasis |
| Nimotuzumab | BioMab-EGFR, | epidermal growth factor | squamous cell carcinoma, head and |
| | Theracim, Theraloc | receptor (EGFR) | neck cancer, nasopharyngeal |
| | | | cancer, glioma |
| Nirsevimab | | RSV fusion glycoprotein | respiratory syncytial virus |
| Nivolumab | Opdivo | PD-1 | melanoma, lung, and renal cancers |
| Nofetumomab | Verluma | | cancer |
| | (Nofetumomab | | |
| | merpentan) | | |
| Obiltoxaximab | Anthim | Bacillus anthracis anthrax | Bacillus anthracis spores |
| Obinutuzumab | Gazyva | CD20 | chronic lymphocytic leukemia |
| Ocaratuzumab | | CD20 | cancer |
| Ocrelizumab | Ocrevus | CD20 | multiple sclerosis |
| Odesivimab | | | Zaire ebolavirus (Ebola virus) |
| Odulimomab | | LFA-1 (CD11a) | prevention of organ transplant |
| | | | rejections, immunological diseases |
| Ofatumumab | Arzerra, Kesimpta | CD20 | chronic lymphocytic leukemia, |
| | | | multiple sclerosis |
| Olaratumab | Lartruvo | PDGFRα | Sarcoma |
| Oleclumab | | 5'-nucleotidase | pancreatic and colorectal cancer |
| Olendalizumab | | complement C5a | systemic lupus |
| | | | erythematosus, lupus nephritis, |
| | | | acute graft-versus-hose disease |
| Olokizumab | | IL6 | rheumatoid arthritis |
| Omalizumab | Xolair | IgE Fc region | allergic asthma |
| Omburtamab | | CD276 | cancer |
| OMS721 | | MASP-2 | atypical hemolytic uremic |
| | | | syndrome |
| Onartuzumab | | human scatter factor | cancer |
| | | receptor kinase | |
| Ontuxizumab | | TEM1 | cancer |
| Onvatilimab | | VISTA (protein) (VSIR) | |
| Opicinumab | | LINGO-1 | multiple sclerosis |
| Oportuzumab | Vicinium | EpCAM | bladder cancer |
| | (Oportuzumab | | |
| | monatox) | | |
| Oregovomab | OvaRex | CA-125 | ovarian cancer |
| Orticumab | | oxLDL | |
| Otelixizumab | | CD3 | diabetes mellitus type 1 |
| Otilimab | | GMCSF | osteoarthritis, rheumatoid arthritis |
| Otlertuzumab | | CD37 | cancer |
| Oxelumab | | OX-40 | asthma |
| Ozanezumab | | NOGO-A | ALS and multiple sclerosis |
| Ozoralizumab | | TNF-α | inflammation |
| Pagibaximab | | lipoteichoic acid | sepsis (Staphylococcus) |
| Palivizumab | Synagis, | F protein of respiratory | respiratory syncytial virus |
| | Abbosynagis | syncytial virus | (prevention) |
| Pamrevlumab | | connective tissue growth | idiopathic pulmonary fibrosis |
| | | factor (CTGF) | (IPF), pancreatic cancer |
| Panitumumab | Vectibix | epidermal growth factor | colorectal cancer |
| | | receptor (EGFR) | |
| Pankomab | | tumor specific | ovarian cancer |
| | | glycosylation of MUC1 | |
| Panobacumab | | Pseudomonas aeruginosa | Pseudomonas aeruginosa infection |
| Parsatuzumab | | EGFL7 | cancer |
| Pascolizumab | | IL-4 | asthma |
| Pasotuxizumab | | folate hydrolase | cancer |
| Pateclizumab | | lymphotoxin alpha (LTA) | TNF |
| Patritumab | | ERBB3 (HER3) | cancer |
| PDR001 | | PD-1 | melanoma |
| Pembrolizumab | Keytruda | PD-1 | melanoma and other cancers |
| Pemtumomab | Theragyn | MUC1 | cancer |
| Penpulimab | | PD-1 | cancer |
| Perakizumab | | IL 17A | arthritis |
| Pertuzumab | Perjeta | HER2 | breast cancer |
| Pexelizumab | | C5 | reduction of side effects of cardiac |
| | | | surgery |
| Pidilizumab | | PD-1 | cancer and infectious diseases |
| Pinatuzumab | | CD22 | cancer |
| Pintumomab | | adenocarcinoma antigen | adenocarcinoma |
| Placulumab | | human TNF | pain and inflammatory diseases |
| Prezalumab | | human TNF | |
| Plozalizumab | | CCR2 | diabetic |
| | | | nephropathy and arteriovenous |
| | | | graft patency |
| Pogalizumab | | tumor necrosis factor | |
| | | receptor (TNFR) | |
| | | superfamily member 4 | |
| Polatuzumab | Polivy | CD79B | B-cell lymphoma |
| | (Polatuzumab | | |
| | vedotin) | | |
| Ponezumab | | human beta-amyloid | Alzheimer's disease |
| Porgaviximab | | Zaire ebolavirus | Ebola virus disease |
| | | glycoprotein | |
| Prasinezumab | | Alpha-synuclein | Parkinson's disease |
| Prezalizumab | | inducible T-cell co- | |
| | | stimulatory | |
| | | ligand (ICOSL) | |
| Priliximab | | CD4 | Crohn's disease, multiple sclerosis |
| Pritoxaximab | | E. coli shiga toxin type-1 | |
| Pritumumab | | vimentin | brain cancer |
| PRO 140 | | CCR5 | HIV infection |
| Quilizumab | | IGHE | asthma |
| Racotumomab | Vaxira | NGNA ganglioside | non-small cell lung cancer |
| Radretumab | | fibronectin extra domain- | cancer |
| | | B | |
| Rafivirumab | | rabies virus glycoprotein | rabies (prophylaxis) |
| Ralpancizumab | | PCSK9 | dyslipidemia |
| Ramucirumab | Cyramza | VEGFR2 | gastric cancer |
| Ranevetmab | | NGF | osteoarthritis in dogs |
| Ranibizumab | Lucentis | VEGF-A | macular degeneration (wet form) |
| Raxibacumab | | anthrax toxin, protective | anthrax (prophylaxis and treatment) |
| | | antigen | |
| Ravagalimab | | CD40 | Crohn's disease |
| Ravulizumab | Ultomiris | C5 | paroxysmal nocturnal |
| | | | hemoglobinuria, atypical hemolytic |
| | | | uremic syndrome |
| Refanezumab | | myelin-associated | recovery of motor function after |
| | | glycoprotein | stroke |
| Regavirumab | | cytomegalovirus glycopro | cytomegalovirus infection |
| | | tein B | |
| Regdanvimab | Regkirona | spike protein receptor | coronavirus disease 2019 (COVID- |
| | | binding domain (RBD) of | 19) |
| | | SARS-CoV-2 | |
| Relatlimab | | LAG3 | melanoma |
| Remtolumab | | interleukin 17 alpha, TNF | |
| Reslizumab | Cinqair | IL-5 | inflammations of the airways, skin |
| | | | and gastrointestinal tract |
| Rilotumumab | | hepatocyte growth | solid tumors |
| | | factor (HGF) | |
| Rinucumab | | platelet-derived growth | neovascular age-related macular |
| | | factor receptor beta | degeneration |
| Risankizumab | Skyrizi | IL23A | Crohn's disease, psoriasis, psoriatic |
| | | | arthritis, and asthma |
| Rituximab | Mab Thera, Rituxan | CD20 | B-Cell Lymphoma |
| Rivabazumab | | Pseudomonas | |
| | | aeruginosa type III | |
| | | secretion system | |
| Robatumumab | | IGF-1 receptor (CD221) | cancer |
| Rmab | RabiShield | rabies virus G | post-exposure prophylaxis of rabies |
| | | glycoprotein | |
| Roledumab | | RHD (gene) (RHD) | Rh disease |
| Romilkimab | | interleukin 13 | |
| Romosozumab | Evenity | sclerostin | osteoporosis |
| Rontalizumab | | IFN-α | systemic lupus erythematosus |
| Rosmantuzumab | | root plate-specific spondin | cancer |
| | | 3 | |
| Rovalpituzumab | | DLL3 | small cell lung cancer |
| Rovelizumab | LeukArrest | CD11, CD18 | haemorrhagic shock etc. |
| Rozanolixizumab | | FCGRT | Immune thrombocytopeni |
| | | | purpura (ITP), myasthenia gravis |
| Ruplizumab | Antova | CD154 (CD40L) | rheumatic diseases |
| SA237 | | IL-6R | neuromyelitis optica and |
| | | | neuromyelitis optica spectrum |
| | | | disorders |
| Sacituzumab | Trodelvy | TROP-2 | triple-negative breast cancer |
| | (Sacituzumab | | |
| | govitecan) | | |
| Samalizumab | | CD200 | cancer |
| Samrotamab | | LRRC15 | cancer |
| Sarilumab | Kevzara | IL6 | rheumatoid arthritis, ankylosing |
| | | | spondylitis |
| Satralizumab | Enspryng | IL6 receptor | neuromyelitis optica |
| Satumomab | | TAG-72 | cancer |
| Secukinumab | Cosentyx | IL 17A | uveitis, rheumatoid |
| | | | arthritis psoriasis |
| Selicrelumab | | CD40 | |
| Seribantumab | | ERBB3 (HER3) | cancer |
| Setoxaximab | | E. coli shiga toxin type-2 | |
| Setrusumab | | sclerostin (SOST) | |
| Sevirumab | | cytomegalovirus | cytomegalovirus infection |
| Sibrotuzumab | | FAP (gene) (FAP) | cancer |
| SGN-CD19A | | CD19 | acute lymphoblastic leukemia and |
| | | | B-cell non-Hodgkin lymphoma |
| SHP647 | | mucosal addressin cell | Crohn's disease |
| | | adhesion molecule | |
| Sifalimumab | | IFN-α | systemic lupus |
| | | | erythematosus (SLE), dermatomyo |
| | | | sitis, polymyositis |
| Siltuximab | Sylvant | IL-6 | cancer |
| Simtuzumab | | LOXL2 | fibrosis |
| Siplizumab | | CD2 | psoriasis, graft-versus-host |
| | | | disease (prevention) |
| Sirtratumab | | SLITRK6 | cancer |
| Sirukumab | | IL-6 | rheumatoid arthritis |
| Sofituzumab | | CA-125 | ovarian cancer |
| Solanezumab | | beta amyloid | Alzheimer's disease |
| Solitomab | | EpCAM | gastrointestinal, lung, and other |
| | | | cancers |
| Sonepcizumab | | sphingosine-1-phosphate | choroidal and retinal neovascularization |
| Sontuzumab | | episialin | |
| Sotrovimab | Xevudy | spike protein receptor | coronavirus disease 2019 (COVID- |
| | | binding domain (RBD) of | 19) |
| | | SARS-CoV-2 | |
| Spartalizumab | | PDCD1, CD279 | melanoma |
| Stamulumab | | myostatin | muscular dystrophy |
| Sulesomab | LeukoScan | NCA-90 (granulocyte | osteomyelitis |
| | | antigen) | |
| Suptavumab | | RSVFR | medically attended lower |
| | | | respiratory disease |
| Sutimlimab | | C1s | cold agglutinin disease |
| Suvizumab | | HIV-1 | viral infections |
| Suvratoxumab | | Staphylococcus | nosocomial pneumonia |
| | | aureus alpha toxin | |
| Tabalumab | | B-cell activating | B-cell cancers |
| | | factor (BAFF) | |
| Tacatuzumab | AFP-Cide | alpha-fetoprotein | cancer |
| | (Tacatuzumab | | |
| | tetraxetan) | | |
| Tadocizumab | | integrin α_{IIb}β₃ | percutaneous coronary intervention |
| Tafasitamab | Monjuvi | CD19 | Diffuse large B-cell lymphoma |
| Talacotuzumab | | CD123 | leukemia |
| Talizumab | | IgE | allergic reaction |
| Talquetamab | | GPRC5D, CD3 | relapsed or refractory multiple |
| | | | myeloma |
| Tamtuvetmab | Tactress | CD52 | |
| Tanezumab | | nerve growth | pain |
| | | factor (NGF) | |
| Taplitumomab | | CD19 | cancer |
| Tarextumab | | Notch receptor | cancer |
| Tavolimab | | CD134 | cancer |
| Teclistamab | | B-cell maturation | relapsed or refractory multiple |
| | | antigen (BCMA), CD3 | myeloma |
| Tefibazumab | Aurexis | clumping factor A | Staphylococcus aureus infection |
| Telimomab | | | |
| aritox | | | |
| Telisotuzumab | | HGFR | cancer |
| Telisotuzumab | | HGFR | cancer |
| Tenatumomab | | tenascin C | cancer |
| Teneliximab | | CD40 | autoimmune diseases and |
| | | | prevention of organ transplant |
| | | | rejection |
| Teplizumab | | CD3 | diabetes mellitus type 1 |
| Tepoditamab | | dendritic cell- | cancer |
| | | associated lectin 2 | |
| Teprotumumab | Tepezza | IGF-1 receptor (CD221) | thyroid eye disease |
| Tesidolumab | | C5 | |
| Tetulomab | | CD37 | cancer |
| Tezepelumab | | thymic stromal | asthma, atopic dermatitis |
| | | lymphopoietin (TSLP) | |
| TGN1412 | | CD28 | chronic lymphocytic |
| | | | leukemia, rheumatoid arthritis |
| Tibulizumab | | B-cell activating | autoimmune disorders |
| | | factor (BAFF) | |
| Tildrakizumab | Ilumya | IL23 | immunologically mediated |
| | | | inflammatory disorders |
| Tigatuzumab | | TRAIL-R2 | cancer |
| Timigutuzumab | | HER2 | cancer |
| Timolumab | | AOC3 | |
| tiragolumab | | | |
| Tiragotumab | | TIGIT | cancer |
| Tislelizumab | | PCDC1, CD279 | non-small cell lung cancer |
| Tisotumab | Tivdak (Tisotumab | coagulation factor III | Cervical cancer |
| | vedotin) | | |
| TNX-650 | | IL-13 | Hodgkin's lymphoma |
| Tocilizumab | Actemra, | IL-6 receptor | rheumatoid arthritis |
| | RoActemra | | |
| Tomuzotuximab | | epidermal growth factor | cancer |
| | | receptor (EGFR), HER1 | |
| Toralizumab | | CD154 (CD40L) | rheumatoid arthritis, lupus |
| | | | nephritis, etc. |
| Toripalimab | Tuoyi | PD-1 | cancer |
| Tosatoxumab | | Staphylococcus aureus | |
| Tositumomab | Bexxar | CD20 | Non-Hodgkin's lymphoma |
| Tovetumab | | PDGFRA | cancer |
| Tralokinumab | Adtralza | IL-13 | atopic dermatitis |
| Trastuzumab | Herceptin, Kadcyla | HER2 | breast cancer |
| | (Trastuzumab | | |
| | duocarmazine), | | |
| | Kadcyla | | |
| | (Trastuzumab | | |
| | emtansine) | | |
| TRBS07 | Ektomab | GD2 ganglioside | melanoma |
| Tregalizumab | | CD4 | |
| Tremelimumab | | CTLA-4 | non-small cell lung, head & neck, |
| | | | urothelial cancer |
| Trevogrumab | | growth differentiation | muscle atrophy due to orthopedic |
| | | factor 8 | disuse and sarcopenia |
| Tucotuzumab | | EpCAM | cancer |
| Tuvirumab | | hepatitis B virus | chronic hepatitis B |
| Ublituximab | | MS4A1 | multiple sclerosis, chronic |
| | | | lymphocytic leukemia |
| Ulocuplumab | | CXCR4 (CD184) | hematologic malignancies |
| Urelumab | | 4-1BB (CD137) | cancer |
| Urtoxazumab | | Escherichia coli | diarrhoea caused by E. coli |
| Ustekinumab | Stelara | IL-12, IL-23 | multiple sclerosis, psoriasis, |
| | | | psoriatic arthritis |
| Utomilumab | | 4-1BB (CD137) | diffuse large B-cell lymphoma |
| Vadastuximab | | CD33 | acute myeloid leukemia |
| Vanalimab | | CD40 | |
| Vandortuzumab | | STEAP1 | cancer |
| Vantictumab | | Frizzled receptor | cancer |
| Vanucizumab | | angiopoietin 2 | cancer |
| Vapaliximab | | AOC3 (VAP-1) | |
| Varisacumab | | VEGF-A | angiogenesis |
| Varlilumab | | CD27 | solid tumors and hematologic |
| | | | malignancies |
| Vatelizumab | | ITGA2 (CD49b) | |
| Vedolizumab | Entyvio | integrin α₄ β₇ | Crohn's disease, ulcerative colitis |
| Veltuzumab | | CD20 | non-Hodgkin's lymphoma |
| Vepalimomab | | AOC3 (VAP-1) | inflammation |
| Vesencumab | | NRP1 | solid malignancies |
| Visilizumab | Nuvion | CD3 | Crohn's disease, ulcerative colitis |
| Vobarilizumab | | IL6R | inflammatory autoimmune diseases |
| Volociximab | | integrin α₅β₁ | solid tumors |
| Vonlerolizumab | | CD134 | cancer |
| Vopratelimab | | CD278, aka ICOS | |
| Vorsetuzumab | | CD70 | cancer |
| Votumumab | HumaSPECT | tumor | colorectal tumors |
| | | antigen CTAA16.88 | |
| Vunakizumab | | interleukin 17 alpha | |
| Xentuzumab | | IGF1, IGF2 | |
| XMAB-5574 | | CD19 | diffuse large B-cell lymphoma |
| Zalutumumab | | Epidermal growth factor | squamous cell carcinoma of the |
| | | receptor (EGFR) | head and neck |
| Zanolimumab | | CD4 | rheumatoid arthritis, psoriasis, T- |
| | | | cell lymphoma |
| Zatuximab | | HER1 | cancer |
| Zenocutuzumab | | ERBB3, HER3 | cancer |
| Ziralimumab | | CD147 (basigin) | |
| Zolbetuximab | | Claudin 18 Isoform 2 | gastric cancer, gastrointestinal |
| | | | adenocarcinoma, and pancreatic |
| | | | cancer |
| Zolimomab | | CD5 | systemic lupus erythematosus, |
| | | | graft-versus-host disease |

In certain embodiments, the antibody is an anti-cancer antibody or antigen-binding fragment thereof. As used herein, “anti-cancer antibody” refers to an antibody that targets an antigen expressed on a cancer cell (e.g., a cancer-associated antigen or cancer-specific antigen), can inhibit the growth of a cancer or tumor, and/or can trigger cancer cell death. In certain embodiments, the antibody is an anti-cancer mAb or an antigen-binding fragment thereof. Table 3 below provides examples of anti-cancer mAbs (including generic name, known target antigen(s), and exemplary use(s)). Other non-limiting examples of anti-cancer mAbs are provided above in Table 2. Therapeutic applications of the anti-cancer mAbs listed below are not limited the particular known target antigen(s) and exemplary use(s) provided.

TABLE 3
Examples of Anti-Cancer Monoclonal Antibodies (mAbs)

| Generic Name | Known Target Antigen(s) | Exemplary Use(s) |
|---|---|---|
| Atezolizumab | PD-L1 | bladder, non-small cell lung, and triple-negative breast |
| | | cancers |
| Avelumab | PD-L1 | urothelial carcinoma and Merkel cell carcinoma |
| Bevacizumab | VEGF | colorectal, non-small cell lung, renal, glioblastoma, and |
| | | ovarian cancers |
| Cemiplimab | PD-1 | cutaneous squamous cell carcinoma |
| Cetuximab | EGFR | colorectal cancer and head and neck squamous cell |
| | | carcinoma |
| Daratumumab | CD38 | multiple myeloma |
| Dinutuximab | GD2 | neuroblastoma |
| Durvalumab | PD-L1 | bladder cancer |
| Elotuzumab | SLAMF7 | multiple myeloma |
| Ipilimumab | CTLA-4 | melanoma and renal cell carcinoma |
| Isatuximab | CD38 | multiple myeloma |
| Mogamulizumab | CCR4 | cutaneous T-cell lymphoma |
| Necitumumab | EGFR | non-small cell lung cancer |
| Nivolumab | PD-1 | melanoma, lung, and renal cancers |
| Obinutuzumab | CD20 | chronic lymphocytic leukemia |
| Ofatumumab | CD20 | chronic lymphocytic leukemia |
| Olaratumab | PDGFRα | sarcoma |
| Panitumumab | EGFR | colorectal cancer |
| Pembrolizumab | PD-1 | melanoma and other cancers |
| Pertuzumab | HER2 | breast cancer |
| Ramucirumab | VEGFR2 | gastric cancer |
| Rituximab | CD20 | B-cell lymphoma |
| Trastuzumab | HER2 | breast cancer |
| Gemtuzumab | CD33 | acute myeloid leukemia |
| Brentuximab | CD30 | Hodgkin's lymphoma and anaplastic large-cell |
| | | lymphoma |
| Inotuzumab | CD22 | acute lymphoblastic leukemia |
| Polatuzumab | CD79B | B-cell lymphoma |
| Enfortumab | Nectin-4 | bladder cancer |
| Sacituzumab | TROP2 | triple negative breast cancer |
| Moxetumomab | CD22 | hairy-cell leukemia |
| Ibritumomab | CD20 | non-Hodgkin's lymphoma |
| tositumomab | CD20 | non-Hodgkin's lymphoma |
| Blinatumomab | CD19, CD3 | acute lymphoblastic leukemia |
| Tisotumab | Tissue factor | cervical cancer |
| Amivantamab | EGFR, cMET | non-small cell lung cancer |
| Loncastuximab | CD19 | diffuse large B-cell lymphoma |
| Dostarlimab | PD-1 | endometrial cancer |
| Margetuximab | HER2 | breast cancer |
| Naxitamab | GD2 | neuroblastoma |
| Belantamab | BCMA | multiple myeloma |
| Tafasitamab | CD19 | diffuse large B-cell lymphoma |

In certain embodiments, the antibody is a homolog of an antibody provided herein, or an antigen-binding fragment thereof. As used herein, the term “homolog” refers to an antibody of similar amino acid composition or sequence to the disclosed antibody, allowing for variations that do not have an adverse effect on the ability of the antibody to carry out its normal function (e.g., binding to a target antigen). Homologs may be the same length, shorter, or longer than the disclosed antibody. Homologs may have at least about 60% (e.g., at least about 60%, at least about 62%, at least about 64%, at least about 66%, at least about 68%, at least about 70%%, at least about 72%, at least about 74% at least about 76%, at least about 78%, at least about 80%, at least about 82%, at least about 84%, at least about 86%, at least about 88%, at least about 90%%, at least about 92%, at least about 94% at least about 96%, at least about 98%, or at least about 99%) sequence identity (i.e., homology) to the amino acid sequence of the disclosed antibody. A homolog can be, for example, an antibody sequence that is modified by deletion, addition, mutation, or substitution of one or more amino acid residues.

In certain embodiments, the antibody is a homolog of trastuzumab, or an antigen-binding fragment thereof. In certain embodiments, the antibody is a homolog of daratumumab, or an antigen-binding fragment thereof.

Antibodies not disclosed herein and/or not yet known in the art may be used in the SPACs provided herein.

In certain embodiments, the antibody is an antibody-drug conjugate (ADC) or antigen-binding fragment thereof. In such embodiments, the SPAC comprises an antibody or antigen-binding fragment thereof conjugated to (i) a stapled peptide or pharmaceutically acceptable salt thereof; and (ii) a second agent (i.e., the “drug” component of the ADC). In other words, a SPAC provided herein can comprise two or more distinct payloads which can be selected from different classes of agents (e.g., with different mechanisms of action). For example, a SPAC provided herein can comprise an anti-cancer stapled peptide or pharmaceutically acceptable salt thereof conjugated to trastuzumab emtansine (i.e., trastuzumab conjugated to (i) the anti-cancer stapled peptide or pharmaceutically acceptable salt thereof; and (ii) emtansine). Table 8 below provides examples of antibody-drug conjugates (ADCs) (including generic name, trade name, and exemplary use(s)). Any of these ADCs may be conjugated to a stapled peptide or pharmaceutically acceptable salt thereof to form a SPAC provided herein. Therapeutic applications of the ADCs listed below are not limited the particular known target antigen(s) and exemplary use(s) provided.

TABLE 8
Examples of Antibody-Drug Conjugates (ADCs)

| | | Trade |
|---|---|---|
| Generic Name | Exemplary Use(s) | Name |
| Gemtuzumab ozogamicin | acute myelogenous leukemia (AML) | Mylotarg |
| Brentuximab vedotin | Hodgkin lymphoma (HL) and systemic anaplastic large-cell | Adcetris |
| | lymphoma (ALCL) | |
| Trastuzumab emtansine | breast cancer (e.g., HER2-positive metastatic breast cancer) | Kadcyla |
| Inotuzumab ozogamicin | acute lymphoblastic leukemia (ALL) | Besponsa |
| Polatuzumab vedotin | diffuse large B-cell lymphoma (DLBCL) | Polivy |
| Enfortumab vedotin | urothelial cancer | Padcev |
| Trastuzumab deruxtecan | breast cancer (e.g., HER2-positive metastatic breast cancer) | Enhertu |
| Sacituzumab govitecan | breast cancer (e.g., metastatic triple-negative breast cancer) | Trodelvy |
| Belantamab mafodotin | multiple myeloma | Blenrep |
| Moxetumomab pasudotox | hairy cell leukemia (HCL) | Lumoxiti |
| Loncastuximab tesirine | large B-cell lymphoma (including diffuse large B-cell | Zynlonta |
| | lymphoma (DLBCL), DLBCL arising from low-grade | |
| | lymphoma, and high-grade B-cell lymphoma) | |
| Tisotumab vedotin-tftv | cervical | Tivdak |

As used herein, “antigen” refers to an agent which is targeted by and binds an antibody. In some instances, antigens trigger the immune system to produce antibodies against the antigens in what is known as an immune response. In certain embodiments, an antigen is expressed in a cell or on the surface of a cell (e.g., a cancer cell). In certain embodiments, an antibody described herein is an antibody directed against a cluster of differentiation (CD) antigen (e.g., CD2, CD3, CD4, CD5, CD6, CD8, CD11, CD11a (LFA-1), CD15, CD18 (ITGB2), CD19, CD20 (MS4A1), CD22, CD23, CD25, CD27, CD28, CD30, CD33, CD37, CD38, CD40, CD41, CD44, CD49b (ITGA2), CD51, CD52, CD54 (ICAM-1), CD56, CD62L, CD70, CD74, CD79B, CD80, CD125, CD140a, CD142, CD147, CD152 (CTLA4), CD154, CD200, CD221, CD240D, CD248, CD257 (BAFF), CD274 (PD-L1), CD276, CD279 (PD-1)). Other examples of antigens (i.e., that an antibody described herein may be directed against) include, but are not limited to, glycoproteins (e.g., TROP2, TPBG, EpCAM, CEA, gpA33, Mucins, TAG-72, CA-IX, CA-125 (MUC16), PSMA, endoglin, fibronectin, MUC1, mucin CanAg, rabies virus glycoprotein), glycolipids (e.g., gangliosides (e.g., GD2, GD3, GM2), myelin-associated glycoprotein, TAG-72, TN-C, TYRP1), carbohydrates (e.g., Lewis-Y²), folate binding proteins (e.g., folate receptor 1, folate receptor alpha), vascular targets (e.g., VEGF, VEGFR, αVβ3, α5β1, VAP-1, VEGF-A, VEGFR-1, VEGFR-2), growth factors (e.g., HGF, IGF-1, NGF, HNGF, TGF-β, TGF-β1, TGF-β2, EGFL7, GDF-8), growth factor receptors (e.g., EGFR/ERBB1/HER1, ERBB2/HER2, ERBB3/HER3, HGFR/c-Met, HGFR, HHGFR, IGF-1 receptor, PDGF-Rα, PDGF-Rβ, EphA3, TRAIL-R1, TRAIL-R2 (DR5), RANKL), stromal and extracellular matrix antigens (e.g., FAP, Tensacin), activin receptor (e.g., ACVR2B), activin receptor-like kinase (e.g., activin receptor-like kinase 2), angiopoietin (e.g., angiopoietin-2, angiopoietin-3), interferons (e.g., INF-α, INF-β, INF-γ), interleukins (e.g., IL 17A, IL 17F, IL20, IL-12, IL-23, IL-13, IL-17, IL-1, IL-22, IL-4, IL-5, IL-6, IL-6 receptor, IL-2, IL-23A, IL-31RA, IL-4, IL-6, IL-9, ILGF2), integrins (e.g., α₄β₇, α₅β₁, α₇β₇, α_{IIb}β₃, αᵥβ₃), complement component (e.g., C5, CFD), chemokines (e.g., CCL11, CCL2 (MCP-1)), chemokine receptors (e.g., CCR2, CCR4, CCR5), Notch receptors (e.g., Notch 1, NRP1), virulence factor (e.g., ClfA), colony stimulating factor (e.g., CSF2), colony stimulating factor receptors (e.g., CSF1R), delta-like ligands (e.g., DLL3, DLL4), Lipopolysaccharides (e.g., endotoxins), human leukocyte antigen (e.g., HLA-DR), heat shock proteins (e.g., Hsp90), SLAM proteins (e.g., SLAMF7), tissue factor pathway inhibitors, tumor necrosis factors (e.g., TNF-α), tumor necrosis factor receptors (TNFR superfamily member 4), microphage migration inhibitory factor, rhesus factor, neurite outgrowth inhibitor, alpha-fetoprotein, amyloid beta, carcinoembryonic antigen (CEA), neural apoptosis-regulated proteinase 1, Ch4D5, CLDN18.2, LOXL2, MSLN, NCA-90, PCSK9, sclerostin, syndecan 1, STEAP1, TSLP, TWEAK receptor, tumor antigen CTAA16.88, nectin-4, and TM4SF1. Other examples of known antigens can be found in, e.g., Table 2 and Table 3 above.

In certain embodiments, the antibody is directed against an antigen expressed on a cancer cell. Different classes of antigens expressed on cancer cells include: (i) “cancer-associated antigens” (CAAs), meaning antigens expressed on cancer cells that can also be present on normal cells; (ii) “cancer-specific antigens” (CSAs), meaning antigens expressed on cancer cells that are not found on normal cells; (iii) “tumor-associated antigens” (TAAs), meaning antigens expressed on solid tumor cells that can also be present on normal cells; and (iv) “tumor-specific antigens” (TSAs), meaning antigens expressed on solid tumor cells that are not found on normal cells. In certain embodiments, the antibody is directed against a cancer-associated antigen. In certain embodiments, the antibody is directed against a cancer-specific antigen. In certain embodiments, the antibody is directed against a tumor-associated antigen. In certain embodiments, the antibody is directed against a tumor-specific antigen.

In certain embodiments, the antibody is directed against a growth factor receptor (e.g., EGFR/ERBB1/HER1, ERBB2/HER2, ERBB3/HER3, HGFR/c-Met, HGFR, HHGFR, IGF-1 receptor, PDGF-Rα, PDGF-Rβ, EphA3, TRAIL-R1, TRAIL-R2 (DR5), RANKL). In certain embodiments, the antibody is an antibody directed against human epidermal growth factor receptor 2 (HER2), or an antigen-binding fragment thereof. In certain embodiments, the antibody is a mAb directed against HER2, or an antigen-binding fragment thereof. In certain embodiments, the antibody is trastuzumab. In certain embodiments, antibody is pertuzumab. In certain embodiments, the antibody is margetuximab.

In certain embodiments, the antibody is an antibody directed against CD38, CD33, CD22, TROP2, CD30, CD79b, or Nectin-4, or antigen-binding fragment thereof. In certain embodiments, the antibody is a mAb directed against CD38, CD33, CD22, TROP2, CD30, CD79b, or Nectin-4, or an antigen-binding fragment thereof.

In certain embodiments, the antibody is an antibody directed against CD38, or an antigen-binding fragment thereof. In certain embodiments, the antibody is a mAb directed against CD38, or an antigen-binding fragment thereof. In certain embodiments, the antibody daratumumab. In certain embodiments, the antibody is isatuximab.

### TM4SF1-Targeting Antibodies

TM4SF1 (Transmembrane-4 L Six Family member 1) is a membrane glycoprotein that was originally discovered on many human epithelial tumor cells. In cancer cells, TM4SF1 is known to support growth, invasion, and metastasis and is therefore a therapeutic target in cancer. See, e.g., Hellström et al., Proc. Natl. Acad. Sci. USA, 1986, 83(18), 7059-7063; Kao et al., Clin. Cancer Res., 2003, 9(7), 2807-2816; Gao et al., Cell, 2016, 166(1), 47-62; Shih et al., Cancer Res., 2009, 69(8), 3272-3277; Lin et al., Angiogenesis, 2014, 17(4), 897-907; and Zukauskas et al., Angiogenesis, 2011, 14(3), 345-354, the entire contents of each of which is incorporated herein by reference.

In certain embodiments, a SPAC provided herein comprises an antibody directed against TM4SF1, or an antigen-binding fragment thereof. In certain embodiments, the antibody is a mAb directed against TM4SF1, or an antigen-binding fragment thereof. In certain embodiments, the antibody or antigen-binding fragment thereof targets the nucleus of a cancer cell (e.g., targets an antigen in the nucleus of a cancer cell). In certain embodiments, the antibody is capable of being internalized to the nucleus of a cancer cell.

Antibodies directed against TM4SF1 are described in, e.g., Sciuto et al., Biochem Biophys Res. Commun., 2015, 465(3), 338-43; and Visintin et al., Mol. Cancer. Ther., 2015, 14(8), 1868-1876. In certain embodiments, the antibody is one disclosed in WO 2020/176794, published Sep. 3, 2020; WO 2021/222783, published Nov. 4, 2021; WO 2021/195598, published Sep. 30, 2021; WO 2019/046338, published Mar. 7, 2019; or WO 2019/241430, published Dec. 19, 2019, the entire contents of each of which is incorporated herein by reference.

### Stapled Peptides

The stapled peptide-antibody conjugates (SPACs) described herein comprise a stapled peptide conjugated to the antibody. The terms “stapled” and “crosslinked” are used interchangeably and refer to peptides wherein two amino acids (i.e., “crosslinked amino acids”) are connected via an internal crosslink (i.e., “staple”) to form a macrocycle. The terms “crosslink” and “staple” are used interchangeably and refer to a covalent linking moiety other than the peptide backbone which connects a pair of crosslinked amino acids to form a macrocycle.

Stapled peptide technology is described in, e.g., U.S. Pat. Nos. 7,192,713; 7,786,072; 8,895,699; 9,505,801; 9,951,099; and 10,487,110, the entire contents of each of which is incorporated herein by reference. Other examples of stapled peptide technology can be found in, e.g., International PCT Application Publication Nos. WO 2017/004591, published Jan. 5, 2017; WO 2019/018499, published Jan. 24, 2019; WO 2021/126827, published Jun. 24, 2021; WO 2014/052647, published Apr. 3, 2014; WO 2014/159969, published Oct. 2, 2014; WO 2011/008260, published Jan. 20, 2011; WO 2009/126292, published Oct. 15, 2009; WO 2013/123266, published Aug. 22, 2013; and WO 2021/188659, published Sep. 23, 2021, the entire contents of each of which are incorporated herein by reference. See also, e.g., Mourtada et al., Nature Biotechnology, 2019, vol. 37, 1186-1197.

Stapled peptides of the disclosure include (i) “singly stapled” peptides, meaning peptides including one internal crosslink connecting two crosslinked amino acids; (ii) “doubly stapled” peptides, meaning peptides including two internal crosslinks, each connecting a different pair of crosslinked amino acids; and (iii) “stitched” peptides, meaning peptides including at least two tandem staples, i.e., staples attached to the same crosslinked amino acid. Stapled peptides can include more than two crosslinks (i.e., multiply stapled), with any number of the staples in the stitched configuration.

In certain embodiments, a crosslink is attached to the α-positions of the crosslinked amino acids. In certain embodiments, crosslinked amino acids are separated by 3 amino acids in the amino acid sequence, forming an “i+4 crosslink.” In certain embodiments, crosslinked amino acids are separated by 4 amino acids in the amino acid sequence, forming an “i+5 crosslink.” In certain embodiments, crosslinked amino acids are separated by 6 amino acids in the amino acid sequence, forming an “i+7 crosslink.” In certain embodiments, crosslinked amino acids are separated by 7 amino acids in the amino acid sequence, forming an “i+8 crosslink.”

Stapling (e.g., crosslinking) a peptide can stabilize a secondary structure (e.g., α-helical secondary structure) of the peptide. In certain embodiments, one or more crosslinks of a stapled peptide provided herein stabilize an α-helix of the peptide. In certain embodiments, a peptide has increased α-helicity as compared to a corresponding unstapled (e.g., uncrosslinked) peptide.

A stapled peptide can exhibit α-helical stability by the maintenance of α-helical structure as measured by circular dichroism or NMR. For example, in certain embodiments, the stapled peptide exhibits at least a 1.1, 1.2, 1.25, 1.3, 1.4, 1.5, 1.6, 1.7, 1.75, 1.8, 1.9, or 2-fold increase in α-helicity (e.g., as determined by circular dichroism or NMR) compared to a corresponding unstapled peptide. In certain embodiments, a stapled peptide provided herein can exhibit about 10%, 15%, 20%, 25%, 30%, 35%, 40%, 45%, 50%, 55%, 60%, 65%, 70%, 75%, 80%, 85%, 90%, 95%, or 100% α-helicity (e.g., as determined by circular dichroism or NMR) compared to a corresponding unstapled peptide.

A stapled peptide can be of any length. In certain embodiments, the stapled peptide is 100 amino acids or fewer in length. In certain embodiments, the stapled peptide is 90 amino acids or fewer in length. In certain embodiments, the stapled peptide is 80 amino acids or fewer in length. In certain embodiments, the stapled peptide is 70 amino acids or fewer in length. In certain embodiments, the stapled peptide is 60 amino acids or fewer in length. In certain embodiments, the stapled peptide is 50 amino acids or fewer in length. In certain embodiments, the stapled peptide is 45 amino acids or fewer in length. In certain embodiments, the stapled peptide is 40 amino acids or fewer in length. In certain embodiments, the stapled peptide is 35 amino acids or fewer in length. In certain embodiments, the stapled peptide is 30 amino acids or fewer in length. In certain embodiments, the stapled peptide is 25 amino acids or fewer in length. In certain embodiments, the stapled peptide is 20 amino acids or fewer in length. In certain embodiments, the stapled peptide is 19, 20, 21, 22, 23, 24, 25, 26, 27, 28, 29, 30, 31, 32, 33, 34, 35, 36, 37, 38, 39, or 40 amino acids or fewer in length. In certain embodiments, the stapled peptide is at least the length of an amino acid sequence provided herein. In certain embodiments, the stapled peptide is the length of any amino acid sequence provided herein.

In certain embodiments, the stapled peptide is conjugated to one or more stapled peptides (e.g., via a linker, such as a cleavable linker). In certain embodiments, the stapled peptide is part of a string of two or more stapled peptides linked to one another in a linear or non-linear fashion (e.g., via linkers, such as cleavable linkers).

In certain embodiments, the stapled peptide is a stapled anti-cancer peptide. “Stapled anti-cancer peptide” (also referred to as “stapled oncolytic peptide”) as used herein refers to a stapled variant of a peptide having anti-cancer activity (i.e., a peptide capable of triggering cancer cell death and/or inhibiting the growth of a cancer or tumor). In certain embodiments, the stapled peptide triggers cancer cell death. Examples of stapled anti-cancer peptides (i.e., stapled oncolytic peptides), any of which can be used as the stapled peptide component of the SPACs provided herein, can be found in International PCT Application Publication Nos. WO 2021/126827, published Jun. 24, 2021, the entire contents of which is incorporated herein by reference.

In certain embodiments, the stapled peptide is a stapled antimicrobial peptide (StAMP). “Stapled anti-microbial peptide” as used herein refers to a stapled variant of a peptide having antimicrobial (e.g., antibacterial) activity. Examples of StAMPs, any of which can be used as the stapled peptide component of the SPACs provided herein, can be found in International PCT Application Publication Nos. WO 2017/004591, published Jan. 5, 2017; and WO 2019/018499, published Jan. 24, 2019, the entire contents of each of which are incorporated herein by reference. A stapled peptide may have both anti-cancer and antimicrobial activity.

### Stapled Peptide Inhibitors of Protein-Protein Interaction (PPI)

In certain embodiments, the stapled peptide is an inhibitor of a protein-protein interaction (PPI). In certain embodiments, the PPI is associated with cancer formation and/or proliferation. See, e.g., Wang et al. Chem. Sci., 2021, 12, 5977; Ali et al., Computational and Structural Biotechnology, Journal, 2019, vol. 17, 263-281; Iyer, Current Medicinal Chemistry, 2016, vol. 23, no. 27, pp. 3025-3043; and Jamieson et al., Reports in Organic Chemistry, 2015, 5, 65-74, the entire contents of each of which is incorporated herein by reference.

### Stapled BCL-2 Family Member Protein Inhibitors

For example, in certain embodiments, the stapled peptide is an inhibitor of a BCL-2 family member protein (e.g., BCL-xL, BCL-2, BCL-W, or MCL1). In certain embodiments, the stapled peptide is an MCL-1 inhibitor (e.g., MCL-1 SAHB_{A}, MCL-1 SAHB_{B}, MCL-1 SAHB_{C}, MCL-1 SAHB_{D}, MCL-1 SAHB_{E}). In certain embodiments, the stapled peptide is a BFL-1 inhibitor. See, e.g., Huhn et al., Cell Chem. Biol., 2016, 23(9): 1123-1134, the entire contents of which is incorporated herein by reference.

In certain embodiments, the stapled peptide is MCL-1 SAHB_{D} as described in, e.g., Stewart et al., Nat. Chem. Biol., 2010, 6(8):595-601, the entire contents of which is incorporated herein by reference. MCL-1 SAHB_{D} comprises the following amino acid sequence:

(SEQ ID NO: 148)
KALETLRRVGDGVXANHXTAF,

wherein both X are connected via the crosslink alk.

In certain embodiments, the stapled peptide is an activator of a BCL-2 family member protein effector (e.g., BAX, BAK, or BOK). In certain embodiments, the stapled peptide is a stapled BH3 peptide. In certain embodiments, the stapled peptide is a stapled BCL-2-interacting mediator of cell death (BIM) peptide. In certain embodiments, the stapled peptide is BIM SAHB_{A1} or BIM SAHB_{A2}. See, e.g., Walensky et al., Science, 2004, 305(5689):1466- 1470; Walensky et al., Mol. Cell., 2006, 24(2):199-210; Gavathiotis et al., Nature, 2008, 455(7216): 1076-1081; Araghi et al., PNAS, 2018, 115(5), E886-E895, the entire contents of each of which is incorporated herein by reference. See also, e.g., International PCT Application Publication Nos. WO 2010/068684, published Jun. 17, 2010; WO 2016/149613, published Sep. 22, 2016; and WO 2017/075349, published May 4, 2017, the entire contents of each of which is incorporated herein by reference. In certain embodiment, the stapled peptide is BID SAHB_{A}. See, e.g., Walensky et al., Molecular Cell, 2006, vol. 24, 199-210. In certain embodiments, the stapled peptide is a stapled PUMA peptide. See, e.g., Edwards et al., Chemistry & Biology, 2013, vol. 20, 888-902.

### Stapled β-Catenin Inhibitors

In certain embodiments, the stapled peptide is a β-catenin inhibitor. In certain embodiments, the stapled peptide is an inhibitor of Wnt/β-catenin signaling. In certain embodiments, the stapled peptide is a stapled BCL9 peptide. See, e.g., Takada et al., Sci. Transl. Med. 2012, 4(148):148ra117, pp. 1-19; Grossman et al., PNAS, 2012, 109(44), 17942-17947; and Liao et al., Cell Discovery, 2020, 6, No. 35, the entire contents of each of which is incorporated herein by reference. See also, e.g., International PCT Application Publication Nos. WO 2012/040459, published Mar. 29, 2012; WO 2012/142604, published Oct. 18, 2012; WO 2019/051327, published Mar. 14, 2019; and WO 2020/041270, published Feb. 7, 2020, the entire contents of each of which is incorporated herein by reference.

For example, in certain embodiments, the stapled peptide is xStAx-34 as described in, e.g., Grossman et al., PNAS, 2012, 109(44), 17942-17947, the entire contents of which is incorporated herein by reference. xStAx-34 comprises the following amino acid sequence: RWPQXILDXHVRRVWR (SEQ ID NO: 149), wherein both X are connected via the crosslink alk.

For example, in certain embodiments, the stapled peptide is SAH-BCL9B as described in, e.g., Takada et al., Sci. Transl. Med. 2012, 4(148):148ra117, pp. 1-19, the entire contents of which is incorporated herein by reference. SAH-BCL9B comprises the following amino acid sequence: LSQEQLEHRERSLXTLRXIQRBLF (SEQ ID NO: 150), wherein both X are connected via the crosslink alk.

### Stapled MDM2 MDMX Inhibitors

In certain embodiments, the stapled peptide is an MDM2 and/or MDMX inhibitor. In certain embodiments, the stapled peptide is an MDM2 inhibitor. In certain embodiments, the stapled peptide is an MDMX inhibitor. In certain embodiments, the stapled peptide inhibits the binding of MDM2 to p53. In certain embodiments, the stapled peptide inhibits the binding of MDMX to p53. In certain embodiments, the stapled peptide is a stapled p53 peptide. See, e.g., Verdine et al., J. Am. Chem. Soc. 2007, 129, 2456-2457; Bernal et al., Cancer Cell, 2010, 18(5):411-422; Chang et al., PNAS, 2013, 110(36), E3445-E3454. See, e.g., International PCT Application Publication Nos. WO 2021/222243, published Nov. 4, 2021; WO 2017/165617, published Sep. 28, 2017; WO 2008/095063, published Aug. 7, 2008; WO 2009/126292, published Oct. 15, 2009; and WO 2013/123266, published Aug. 22, 2013, the entire contents of each of which is incorporated herein by reference. See, e.g., U.S. Pat. Nos. 7,192,713; 7,786,072; 8,895,699; 9,505,801; 9,951,099; and 10,487,110, the entire contents of each of which is incorporated herein by reference. In certain embodiments, the stapled peptide is ALRN-6924 or ATSP-7041. See, e.g., Ye Che, Methods Mol. Biol., 2019, 2001, 97-106; and Chang et al., PNAS, 2013, 110(36), E3445-E3454, the entire contents of each of which are incorporated herein by reference.

The structures of ALRN-6924 and ATSP-7041 are shown below:

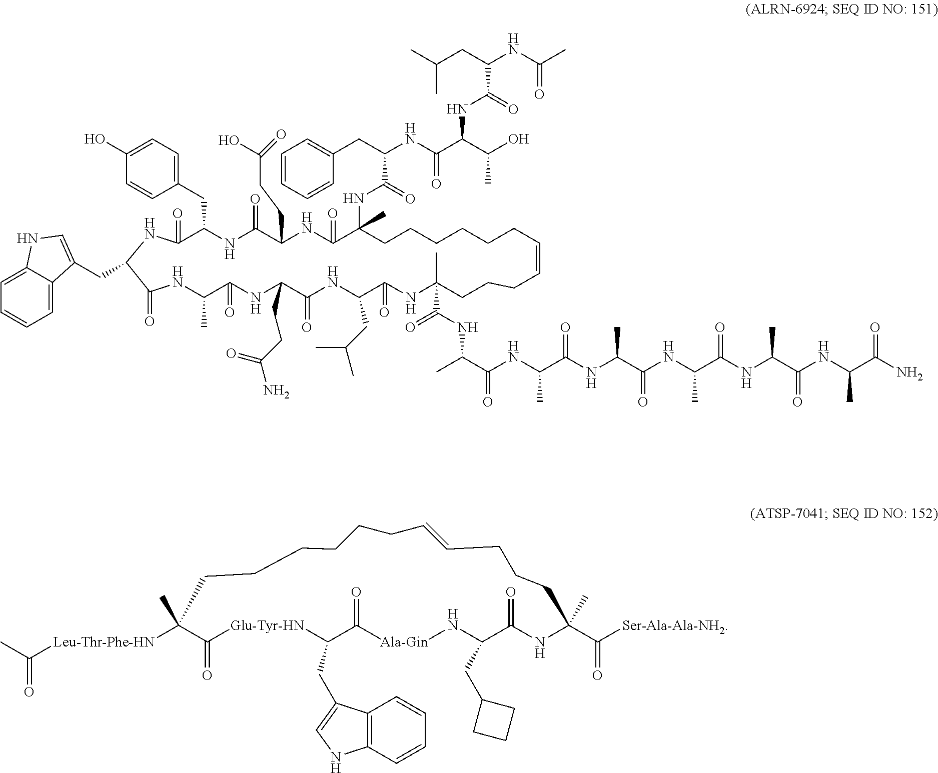

In certain embodiments, the stapled peptide is ATSP-7041 wherein the cyclobutylalanine amino acid (Cba) is substituted by leucine (L) (i.e., SEQ ID NO: 152 with a Cba10L amino acid substitution) (referred to herein as “ATSP-7041 Cba10L”). In certain embodiments, the stapled peptide comprises one of the following amino acid sequences (wherein both X are amino acids connected via a crosslink):

(SEQ ID NO: 161)
LTFXEYWAQLXSAA,
(SEQ ID NO: 162)
LTFXEYWAQLXSAAGGG,
(SEQ ID NO: 163)
LTFXEYWAQLXSAA-PEG3,

or a pharmaceutically acceptable salt thereof.

In certain embodiments, the stapled peptide comprises one of the following (wherein R⁸ and S⁵ are joined together to form the crosslink alk2):

(SEQ ID NO: 164)
LTFR⁸EYWAQLS⁵SAA—NH₂,
(SEQ ID NO: 165)
LTFR⁸EYWAQLS⁵SAAGGG—NH₂,
(SEQ ID NO: 166)
LTFR⁸EYWAQLS⁵SAA-PEG3—NH₂,

or a pharmaceutically acceptable salt thereof.

### Stapled Magainin Peptides

In certain embodiments, the stapled peptide is based on the amino acid sequence of a Magainin peptide (e.g., Magainin II). The Magainins are a class of antimicrobial peptides (AMPs) originally found in the African clawed frog (Xenopus laevis). The peptides are cationic, generally lack a stable conformation in water but form amphipathic α-helices in membranes. They are generally known to disrupt the cell membranes of a broad spectrum of cells, including bacteria, protozoa, and fungi. They have also been reported to have anti-cancer activity. The amino acid sequences of the peptides known as “Magainin I” and “Magainin II” are provided below.

| | | |
|---|---|---|
| Magainin I: | GIGKFLHSAGKFGKAFVGEIMKS | (SEQ ID NO: 153) |
| Magainin II: | GIGKFLHSAKKFGKAFVGEIMNS | (SEQ ID NO: 154) |

Examples of stapled Magainin peptides, any of which can be used as the stapled peptide component of the SPACs provided herein, can be found in International PCT Application Publication Nos. WO 2017/004591, published Jan. 5, 2017; WO 2019/018499, published Jan. 24, 2019; and WO 2021/126827, published Jun. 24, 2021, the entire contents of each of which are incorporated herein by reference. See also, e.g., U.S. Provisional Application, U.S. Ser. No. 63/160,245, filed Mar. 12, 2021, the entire contents of which is incorporated herein by reference. See also, e.g., Mourtada et al., Nature Biotechnology, 2019, vol. 37, 1186-1197, the entire contents of which is incorporated herein by reference.

In certain embodiments, the stapled peptide is a stapled Magainin peptide. In certain embodiments, the stapled peptide is a stapled Magainin II peptide.

In certain embodiments, the stapled peptide comprises the amino acid sequence:

(SEQ ID NO: 1)
G X¹ G K F X² H S K K K F G K A X³ V G E X⁴,

or a pharmaceutically acceptable salt thereof, wherein:
X¹, X², X³, and X⁴ are amino acids (i.e., crosslinked amino acids);
X¹ and X² are connected via a crosslink, and X³ and X⁴ are connected via a crosslink; and
the amino acid sequence includes 0 to 9 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions. In certain embodiments, the amino acid sequence comprises 9 amino acid substitutions.

In certain embodiments, the stapled peptide comprises the amino acid sequence:

(SEQ ID NO: 2)
G X¹ G K F X² H S K K K F G K A X³ V G E X⁴ A K K,

or a pharmaceutically acceptable salt thereof, wherein:
X¹, X², X³, and X⁴ are amino acids (i.e., crosslinked amino acids);
X¹ and X² are connected via a crosslink, and X³ and X⁴ are connected via a crosslink; the amino acid sequence includes 0 to 11 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions. In certain embodiments, the amino acid sequence comprises 9 amino acid substitutions.

In certain embodiments, a stapled peptide or pharmaceutically acceptable salt thereof comprises one of the following amino acid sequences:

(SEQ ID NO: 3)
G X¹ G K F X² K S K K K F G K A X³ V G E X³ A K K,
(SEQ ID NO: 4)
G X¹ G K F X² H K K K K F G K A X³ V G E X³ A K K,
(SEQ ID NO: 5)
G X¹ G K F X² K K K K K F G K A X³ V G E X³ A K K,
(SEQ ID NO: 6)
G X¹ G K F X² H K K K K F G K A X³ V G E X³ A K K,
(SEQ ID NO: 7)
G X¹ G K F X² H N K K K F G K A X³ V G E X³ A K K,
(SEQ ID NO: 8)
G X¹ G K F X² H Q K K K F G K A X³ V G E X³ A K K,
(SEQ ID NO: 9)
G X¹ G K F X² H T K K K F G K A X³ V G E X³ A K K,
(SEQ ID NO: 10)
G X¹ G K F X² H Y K K K F G K A X³ V G E X³ A K K,
(SEQ ID NO: 11)
G X¹ G K F X² H S K K K F G K A X³ V W E X³ A K K,
(SEQ ID NO: 12)
G X¹ G K F X² H S K K K F G K A X³ V G E X³ A K K,
(SEQ ID NO: 13)
G X¹ G K F X² H S K K K F G K A X³ V L E X³ A K K,
(SEQ ID NO: 14)
G X¹ G K F X² H S K K K F G K A X³ V Y E X³ A K K,
(SEQ ID NO: 15)
G X¹ G K F X² H S K K K F G K A X³ V F E X³ A K K,
(SEQ ID NO: 16)
G X¹ G K F X² H S K K K F G K A X³ V T E X³ A K K,
(SEQ ID NO: 17)
G X¹ G K F X² H S K K K F G K A X³ V G D X³ A K K,
(SEQ ID NO: 18)
G X¹ G K F X² H S K K K F G K A X³ V G Q X³ A K K,
(SEQ ID NO: 19)
G X¹ G K F X² H S K K K F G K A X³ V G N X³ A K K,
(SEQ ID NO: 20)
G X¹ G K F X² K S K K K F G K A X³ V V E X³ A K K,
(SEQ ID NO: 21)
G X¹ G K F X² K S K K K F G K A X³ V F E X³ A K K,
(SEQ ID NO: 22)
G X¹ G K F X² H K K K K F G K A X³ V V E X³ A K K,
(SEQ ID NO: 23)
G X¹ G K F X² H K K K K F G K A X³ V F E X³ A K K,
(SEQ ID NO: 24)
G X¹ G Dab F X² Dab Dab Dab Dab Dab F G Dab A X³ V G E X³ A Dab
Dab,
(SEQ ID NO: 25)
G X¹ G Dab F X² Orn Orn Orn Orn Orn F G Orn A X³ V G E X³ A Orn
Orn,
(SEQ ID NO: 26)
G X¹ G Dap F X² Dap Dap Dap Dap Dap F G Dap A X³ V G E X³ A Dap
Dap,
(SEQ ID NO: 27)
G X¹ G K F X² K K K K K F G K A X³ V G E X³ A K K G G E,
(SEQ ID NO: 28)
G X¹ G K F X² K K K K K F G K A X³ V G E X³ K,
(SEQ ID NO: 29)
G X¹ G K F¹ X² H K K K K F¹ G K A X³ V V E X³ A K K,
(SEQ ID NO: 30)
G X¹ G K F³ X² H K K K K F³ G K A X³ V V E X³ A K K,
(SEQ ID NO: 31)
G X¹ G K F5 X² H K K K K F5 G K A X³ V V E X³ A K K,
(SEQ ID NO: 32)
G X¹ G K F² X² H K K K K F² G K A X³ V V E X³ A K K,
(SEQ ID NO: 33)
G X¹ G K F³ X² H K K K K F³ G K A X³ V V E X³ A K K,
(SEQ ID NO: 34)
G X¹ G K F X² K K K K K F G K A X³ V V E X³ A K K G G E,
(SEQ ID NO: 35)
G X¹ G K F X² K K K K K F G K A X³ V F E X³ A K K G G E,
(SEQ ID NO: 36)
G X¹ G K F X² K K K K K F G K A X³ V F¹ E X³ A K K G G E,
(SEQ ID NO: 37)
G X¹ G K F X² K K K K K F G K A X³ V F² E X³ A K K G G E,
(SEQ ID NO: 38)
G X¹ G K F X² K K K K K F G K A X³ V F³ E X³ A K K G G E,
(SEQ ID NO: 39)
G X¹ G Dab F X² K K K K K F G K A X³ V G E X³ A K K,
(SEQ ID NO: 40)
G X¹ G K F X² K Dab K K K F G K A X³ V G E X³ A K K,
(SEQ ID NO: 41)
G X¹ G K F X² K K K K K F G Dap A X³ V G E X³ A K K,
(SEQ ID NO: 42)
G X¹ G K F X² K Dap K K K F G K A X³ V G E X³ A K K,
(SEQ ID NO: 43)
G X¹ G K F X² Dap K K K K F G K A X³ V G E X³ A K K,
(SEQ ID NO: 44)
G X¹ G K F X² K Dap K K K F G K A X³ V G E X³ A K K,
(SEQ ID NO: 45)
G X¹ G K F X² K K Dap K K F G K A X³ V G E X³ A K K,
(SEQ ID NO: 46)
G X¹ G K F X² K K K K K F G K A X³ V G E X³ A Dap K,
(SEQ ID NO: 47)
G X¹ G Dap F X² K K K K K F G K A X³ V G E X³ A K K,
(SEQ ID NO: 48)
G X¹ G K F X² Dap K K K K F G K A X³ V G E X³ A K K,
(SEQ ID NO: 49)
G X¹ G K F X² K Dap K K K F G K A X³ V G E X³ A K K,
(SEQ ID NO: 50)
G X¹ G K F X² K K K K K F G K A X³ V G E X³ A K K,
(SEQ ID NO: 51)
G X¹ G K F X² K K K K Dap F G K A X³ V G E X³ A K K,
(SEQ ID NO: 52)
G X¹ G K F X² K K K K K F G Dap A X³ V G E X³ A K K,
(SEQ ID NO: 53)
G X¹ G K F X² K K K K K F G K A X³ V G E X³ A Dap K,
(SEQ ID NO: 54)
G X¹ G K F X² K K K K K F G K A X³ V G E X³ A K Dap,
(SEQ ID NO: 55)
G X¹ G K F X² K K K K K F G K A X³ V G E X³ A K K G E
(SEQ ID NO: 56)
G X¹ G K F X² K K K K K F G K A X³ V G E X³ A K K G G G E,
(SEQ ID NO: 57)
G X¹ G K F X² K K K K K F G K A X³ V G E X³ A K K G G E E,
(SEQ ID NO: 168)
G X¹ G K F X² K K K K K F G K A X³ V G E X³ A K K G G Q,
(SEQ ID NO: 169)
G X¹ G Dap F X² Dap Dap Dap Dap Dap F G Dap A X³ V G E X³ A K K,
(SEQ ID NO: 170)
G X¹ G K F X² Dab K K K K F G K A X³ V G E X³ A K K,
(SEQ ID NO: 171)
G X¹ G K F X² Dap K K K K F G K A X³ V G E X³ A K K G G E,
(SEQ ID NO: 172)
G X¹ G K F X² K K Dap K K F G K A X³ V G E X³ A K K G G E,
(SEQ ID NO: 173)
G X¹ G K F X² Dap K Dap K K F G K A X³ V G E X³ A K K,
(SEQ ID NO: 174)
G X¹ G K F X² Dap K K K K F G K A X³ V G E X³ A K K G G E,
(SEQ ID NO: 175)
J X¹ G K F X² Dab Dab Dab Dab Dab F G K A X³ V G E X³ A Dab Dab,
(SEQ ID NO: 176)
G X¹ G K F X² Dab K K K K F G K A X³ V G E X³ A K K,
(SEQ ID NO: 177)
G X¹ G K F X² Dab K K K K F G K A X³ V G E X³ A K K,
(SEQ ID NO: 178)
G X¹ G K F X² Dab K K K K F G K A X³ V G E X³ A K K,
(SEQ ID NO: 179)
G X¹ G K F X² Dab K K K K F G K A X³ V G E X³ A K K,
(SEQ ID NO: 180)
G X¹ G K F X² Dab K K K K F G K A X³ V G E X³ A K K,
(SEQ ID NO: 181)
G X¹ G K F X² Dap K K K K F G K A X³ V G E X³ A K K G G E,
(SEQ ID NO: 182)
G X¹ G K F X² Dap K K K K F G K A X³ V G E X³ A K K G G E,
(SEQ ID NO: 183)
G X¹ G K F X² Dap K K K K F G K A X³ V G E X³ A K K G G E,
(SEQ ID NO: 184)
G X¹ G K F X² Dap K K K K F G K A X³ V G E X³ A K K G G E,
(SEQ ID NO: 185)
G X¹ G K F X² Dap K K K K F G K A X³ V G E X³ A K K G G E,
or
(SEQ ID NO: 186)
G X¹ G K F X² Dap K K K K F G K A X³ V G E X³ A K K G G E J.

In certain embodiments, the stapled peptide is of one of SEQ ID NOs: 1-57 and 167-186, or a pharmaceutically acceptable salt thereof. In certain embodiments, the stapled peptide is of one of SEQ ID NOs: 1-57 and 167-186, or a pharmaceutically acceptable salt thereof, wherein the C-terminus of the peptide is amidated with —NH₂. In certain embodiments, the stapled peptide is of one of SEQ ID NOs: 1-57 and 167-186, or a pharmaceutically acceptable salt thereof, wherein the C-terminus of the peptide is amidated with —NH₂; and wherein X¹ and X², and X³ and X⁴, are each connected via the crosslink (alk).

In certain embodiments, the stapled peptide comprises the amino acid sequence:

(SEQ ID NO: 48)
G X¹ G K F X² Dap K K K K F G K A X³ V G E X⁴ A K K,

or a pharmaceutically acceptable salt thereof. In certain embodiments, the stapled peptide or pharmaceutically acceptable salt thereof is of SEQ ID NO: 48, wherein X¹ and X², and X³ and X⁴, are connected via a crosslink of the formula (alk); and wherein the C-terminus of the stapled peptide is amidated with —NH₂.

In certain embodiments, the stapled peptide comprises the amino acid sequence:

(SEQ ID NO: 26)
G X¹ G Dap F X² Dap Dap Dap Dap Dap F G Dap A X³
V G E X⁴ A Dap Dap,

or a pharmaceutically acceptable salt thereof. In certain embodiments, the stapled peptide or pharmaceutically acceptable salt thereof is of SEQ ID NO: 26, wherein X¹ and X², and X³ and X⁴, are connected via a crosslink of the formula (alk); and wherein the C-terminus of the stapled peptide is amidated with —NH₂.

In certain embodiments, the stapled peptide comprises the amino acid sequence:

(SEQ ID NO: 5)
G X¹ G K F X² K K K K K F G K A X³ V G E X⁴ A K K,

or a pharmaceutically acceptable salt thereof. In certain embodiments, the stapled peptide or pharmaceutically acceptable salt thereof is of SEQ ID NO: 5, wherein X¹ and X², and X³ and X⁴, are connected via a crosslink of the formula (alk); and wherein the C-terminus of the stapled peptide is amidated with —NH₂.

In certain embodiments, the stapled peptide comprises the amino acid sequence:

(SEQ ID NO: 23)
G X¹ G K F X² H K K K K F G K A X³ V F E X4 A K K,

or a pharmaceutically acceptable salt thereof. In certain embodiments, the stapled peptide or pharmaceutically acceptable salt thereof is of SEQ ID NO: 23, wherein X¹ and X², and X³ and X⁴, are connected via a crosslink of the formula (alk); and wherein the C-terminus of the stapled peptide is amidated with —NH₂.

In certain embodiments, the stapled peptide comprises the amino acid sequence:

(SEQ ID NO: 24)
G X¹ G Dab F X² Dab Dab Dab Dab Dab F G Dab A X³ V
G E X⁴ A Dab Dab,

or a pharmaceutically acceptable salt thereof. In certain embodiments, the stapled peptide or pharmaceutically acceptable salt thereof is of SEQ ID NO: 24, wherein X¹ and X², and X³ and X⁴, are connected via a crosslink of the formula (alk); and wherein the C-terminus of the stapled peptide is amidated with —NH₂. In certain embodiments, a stapled peptide or pharmaceutically acceptable salt thereof provided herein comprises one of the following amino acid sequences:

(SEQ ID NO: 58)
W X¹ G K F X² H S K K K F G K A X³ V G E X⁴ A K K,
(SEQ ID NO: 59)
W X¹ G K F X² H S K K K F G K A X³ V G E X⁴ A K K,
(SEQ ID NO: 60)
W X¹ G K F X² H S K K K F G K A X³ V G E X⁴ A K K,
(SEQ ID NO: 61)
W X¹ G K F X² H S K K K F G K A X³ V G E X⁴ A K K,
(SEQ ID NO: 62)
W X¹ G K F X² H S K K K F G K A X³ V G E X⁴ A K K,
(SEQ ID NO: 63)
W X¹ G K F X² H S K K K F G K A X³ V G E X⁴ A K K,
(SEQ ID NO: 64)
W X¹ G K F X² H S K K K W W K A X³ V G E X⁴ A K K,
(SEQ ID NO: 65)
W X¹ G K F X² H S K K K F G K A X³ V G E X⁴ A K K,
(SEQ ID NO: 66)
W X¹ G K F X² H S K K K F G K A X³ V G E X⁴ A K K,
(SEQ ID NO: 67)
W X¹ G K F X² H S K K K F G K A X³ V G E X⁴ A K K,
(SEQ ID NO: 68)
W X¹ G K F X² H S K K K F G K A X³ V G E X⁴ A K K,
(SEQ ID NO: 69)
W X¹ G K F X² H S K K K F G K A X³ V G E X⁴ A K K,
(SEQ ID NO: 70)
W X¹ G K F X² H S K K K W G K A X³ V G E X⁴ A K K,
or
(SEQ ID NO: 71)
W X¹ G K F X² H S K K K F W K A X³ V G E X⁴ A K K.

In certain embodiments, the stapled peptide is of one of SEQ ID NOs: 58-71, or a pharmaceutically acceptable salt thereof. In certain embodiments, the stapled peptide is of one of SEQ ID NOs: 58-71, or a pharmaceutically acceptable salt thereof, wherein the C-terminus is amidated with —NH₂. In certain embodiments, the stapled peptide is of one of SEQ ID NOs: 58-71, or a pharmaceutically acceptable salt thereof, wherein the C-terminus of the peptide is amidated with —NH₂; and wherein X¹ and X², and X³ and X⁴, are each connected via the crosslink (alk).

In certain embodiments, the stapled peptide comprises the following amino acid sequence:

(SEQ ID NO: 72)
G X¹ G K F X² K K K K K X³ V G E X⁴ A K K,

or a pharmaceutically acceptable salt thereof, wherein:
X¹, X², X³, and X⁴ are amino acids (i.e., crosslinked amino acids);
X¹ and X² are connected via a crosslink, and X³ and X⁴ are connected via a crosslink; and
the amino acid sequence optionally includes 0 to 5 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions.

In certain embodiments, the stapled peptide or a pharmaceutically acceptable salt thereof comprises SEQ ID NO: 72. In certain embodiments, the stapled peptide is of SEQ ID NO: 72, or a pharmaceutically acceptable salt thereof. In certain embodiments, the stapled peptide is of SEQ ID NO: 72, or a pharmaceutically acceptable salt thereof, wherein the C-terminus is amidated with —NH₂. In certain embodiments, the stapled peptide is of SEQ ID NO: 72, or a pharmaceutically acceptable salt thereof, wherein the C-terminus of the peptide is amidated with —NH₂; and wherein X¹ and X², and X³ and X⁴, are each connected via the crosslink (alk).

### Stapled Esculentin Peptides

In certain embodiments, the stapled peptide is based on the amino acid sequence of an Esculentin peptide (e.g., Esculentin-1A). Esculentins are a class of cytotoxic peptides with antibacterial and antifungal activity that were originally found in the skin secretions of many species of frogs and toads. Esculentin-1A is a particular antimicrobial peptide (AMP) originally isolated from frog skin. Esculentin peptides can also have anti-cancer activity. The amino acid sequence of the peptide known as “Esculentin-1A” is provided below. In certain embodiments, a stapled peptide is based on amino acids 1-21 of SEQ ID NO: 155.

Esculentin-1A:
(SEQ ID NO: 155)
GIFSKLAGKKIKNLLISGLKNVG
KEVGMDVVRTGIDIAGCKIKGEC

In certain embodiments, the stapled peptide is a stapled Esculentin peptide. In certain embodiments, the stapled peptide is a stapled Esculentin-1A peptide. Examples of stapled Esculentin peptides, any of which can be used as the stapled peptide component of the SPACs provided herein, can be found in, e.g., U.S. Provisional Applications, U.S. Ser. No. 63/185,641, filed May 7, 2021; and 63/185,673, filed May 7, 2021, the entire contents of each of which are incorporated herein by reference. See also, e.g., Mourtada, et al., Nature Biotechnology, 2019, vol. 37, 1186-1197, the entire contents of which is incorporated herein by reference.

In certain embodiments, the stapled peptide comprises the amino acid sequence:

(SEQ ID NO: 73)
G X¹ F S K X² K G K K I K N L X³ I S G X K G,

or a pharmaceutically acceptable salt thereof, wherein:
X¹, X², X³, and X⁴ are amino acids (i.e., crosslinked amino acids);
X¹ and X² are connected via a crosslink, and X³ and X⁴ are connected via a crosslink; and
the amino acid sequence includes 0 to 9 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions. In certain embodiments, the amino acid sequence comprises 9 amino acid substitutions.

In certain embodiments, the stapled peptide or pharmaceutically acceptable salt thereof comprises one of the following amino acid sequences:

(SEQ ID NO: 74)
G X¹ K S K X² K G K K I K N L X³ I S G X⁴ K G,
(SEQ ID NO: 75)
G X¹ F S K X² K G K K I K N L X³ I S K X⁴ K G,
(SEQ ID NO: 76)
G X¹ F S K X² K G K K I K N L X³ I S G X⁴ K K,
(SEQ ID NO: 77)
G X¹ K S K X² K G K K I K N L X³ I S K X⁴ K G,
(SEQ ID NO: 78)
G X¹ K V K X² K G K K I K N L X³ I S G X⁴ K G,
(SEQ ID NO: 79)
G X¹ K S K X² K V K K I K N L X³ I S G X⁴ K G,
(SEQ ID NO: 80)
G X¹ K S K X² K G K K V K N L X³ I S G X⁴ K G,
(SEQ ID NO: 81)
G X¹ K S K X² A G K K L K N L X³ I S G X⁴ K N,
(SEQ ID NO: 82)
G X¹ F S K X² A G K K I K N L X³ I S G X⁴ K N,
(SEQ ID NO: 83)
G X¹ K S K X² A G K K I K N L X³ I S G X⁴ K N,
(SEQ ID NO: 84)
G X¹ K S K X² A G K K L K N L X³ I S G X⁴ K G,
(SEQ ID NO: 85)
G X¹ F S K X² A G K K L K N L X³ I S G X⁴ K N,
(SEQ ID NO: 86)
G X¹ F S Dab X² Dab G Dab Dab I Dab N L X³ I S G
X⁴ Dab G,
(SEQ ID NO: 87)
G X¹ F S Orn X² Orn G Orn Orn I Orn N L X³ I S G
X⁴ Orn G,
(SEQ ID NO: 88)
G X¹ F S Dap X² Dap G Dap Dap I Dap N L X³ I S G
X⁴ Dap G,
(SEQ ID NO: 89)
G X¹ K S K X² K G K K I K N L X³ I S G X⁴ K N,
(SEQ ID NO: 90)
G X¹ K S K X² K G K K F3 K N L X³ I S G X⁴ K N,
(SEQ ID NO: 91)
G X¹ K S K X² K G K K I K N F3 X³ I S G X⁴ K N,
(SEQ ID NO: 92)
G X¹ K S K X² K G K K I K N L X³ F³ S G X⁴ K N,
(SEQ ID NO: 93)
G X¹ K S K X² K G K K I K N L X³ I S V X⁴ K N,
(SEQ ID NO: 94)
G X¹ K S K X² K G K K I K N L X³ I S F X⁴ K N,
(SEQ ID NO: 95)
G X¹ K S K X² K G K K I K N L X³ I S F X⁴ K N E,
or
(SEQ ID NO: 96)
G X¹ K S K X² K G K K I K N L X³ I S F X⁴ K N G G
G E.

In certain embodiments, the stapled peptide is of one of SEQ ID NOs: 73-96, or a pharmaceutically acceptable salt thereof. In certain embodiments, the stapled peptide is of one of SEQ ID NOs: 73-96, or a pharmaceutically acceptable salt thereof, wherein the C-terminus of the peptide is amidated with —NH₂. In certain embodiments, the stapled peptide is of one of SEQ ID NOs: 73-96, or a pharmaceutically acceptable salt thereof, wherein the C-terminus of the peptide is amidated with —NH₂; and wherein X¹ and X², and X³ and X⁴, are each connected via the crosslink (alk).

In certain embodiments, the stapled peptide or pharmaceutically acceptable salt thereof comprises SEQ ID NO: 73. In certain embodiments, the stapled peptide is of SEQ ID NO: 73, or a pharmaceutically acceptable salt thereof. In certain embodiments, the stapled peptide is of SEQ ID NO: 73, or a pharmaceutically acceptable salt thereof, wherein the C-terminus is amidated with —NH₂. In certain embodiments, the stapled peptide is of SEQ ID NO: 73, or a pharmaceutically acceptable salt thereof, wherein the C-terminus of the peptide is amidated with —NH₂; and wherein X¹ and X², and X³ and X⁴, are each connected via the crosslink (alk).

In certain embodiments, the stapled peptide or pharmaceutically acceptable salt thereof provided herein comprises one of the following amino acid sequences:

(SEQ ID NO: 97)
K X¹ F S K X² K G K K I K N L X³ I S G X⁴ K G,
(SEQ ID NO: 98)
G X¹ F K K X² K G K K I K N L X³ I S G X⁴ K G,
(SEQ ID NO: 99)
G X¹ F S K X² K K K K I K N L X³ I S G X⁴ K G,
(SEQ ID NO: 100)
G X¹ F S K X² K G K K K K N L X³ I S G X⁴ K G,
(SEQ ID NO: 101)
G X¹ F S K X² K G K K I K K L X³ I S G X⁴ K G,
(SEQ ID NO: 102)
G X¹ F S K X² K G K K I K N K X³ I S G X⁴ K G,
(SEQ ID NO: 103)
G X¹ F S K X² K G K K I K N L X³ K S G X⁴ K G,
or
(SEQ ID NO: 104)
G X¹ F S K X² K G K K I K N L X³ I K G X⁴ K G.

In certain embodiments, the stapled peptide is of one of SEQ ID NOs: 97-104, or a pharmaceutically acceptable salt thereof. In certain embodiments, the stapled peptide is of one of SEQ ID NOs: 97-104, or a pharmaceutically acceptable salt thereof, wherein the C-terminus is amidated with —NH₂. In certain embodiments, the stapled peptide is of one of SEQ ID NOs: 97-104, or a pharmaceutically acceptable salt thereof, wherein the C-terminus is amidated with —NH₂; and wherein X¹ and X², and X³ and X⁴, are each connected via the crosslink (alk).

In certain embodiments, the stapled peptide comprises the amino acid sequence:

(SEQ ID NO: 105)
G X¹ F S K X² K G K K I K N L L X³ S G L X⁴ G,

and pharmaceutically acceptable salts thereof, wherein:
X¹, X², X³, and X⁴ are amino acids (i.e., crosslinked amino acids);
X¹ and X² are connected via a crosslink, and X³ and X⁴ are connected via a crosslink; and
the amino acid sequence optionally includes 0 to 8 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions.

In certain embodiments, the stapled peptide comprises one of the following amino acid sequences:

(SEQ ID NO: 106)
G X¹ F S K X² K G K K I K N X³ L I S X⁴ L K G,
(SEQ ID NO: 107)
G X¹ F S K X² K G K K I K X³ L L I X⁴ G L K G,
(SEQ ID NO: 108)
G X¹ F S K X² K G K K X³ K N L X⁴ I S G L K G,
(SEQ ID NO: 109)
G I F S K L X¹ G K K X² K N L L I S G L K G,
(SEQ ID NO: 110)
G X¹ F S K X² K G K K X³ I S G X⁴ K G,

or a pharmaceutically acceptable salt thereof, wherein:
X¹, X², X³, and X⁴ are amino acids (i.e., crosslinked amino acids);
X¹ and X² are connected via a crosslink, and X³ and X⁴ are connected via a crosslink; and
the amino acid sequence optionally includes 0 to 8 amino acid substitutions, inclusive, at positions other than X¹, X², X³, and X⁴. In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, the amino acid sequence comprises 7 amino acid substitutions. In certain embodiments, the amino acid sequence comprises 8 amino acid substitutions.

In certain embodiments, the stapled peptide or pharmaceutically acceptable salt thereof comprises one of the following amino acid sequences:

(SEQ ID NO: 111)
G X¹ F S K X² K G K K I K N L L X³ S G K X⁴ G,
(SEQ ID NO: 112)
G X¹ F S K X² K G K K I K N L L X³ S G L X⁴ K,
(SEQ ID NO: 113)
G X¹ F S K X² K G K K I K N L L X³ S G L X⁴ K G
G E,
or
(SEQ ID NO: 114)
G I F S K L X¹ G K K X² K N L K I S G L K G.

In certain embodiments, the stapled peptide or pharmaceutically acceptable salt thereof is of one of SEQ ID NOs: 105-114. In certain embodiments, a stapled peptide or pharmaceutically acceptable salt thereof is of one of SEQ ID NOs: 105-114, wherein the C-terminus is amidated with —NH₂. In certain embodiments, a stapled peptide or pharmaceutically acceptable salt thereof is of one of SEQ ID NOs: 105-114, wherein the C-terminus is amidated with —NH₂; and wherein X¹ and X², and X³ and X⁴, are each connected via the crosslink (alk).

In certain embodiments, the stapled peptide or pharmaceutically acceptable salt thereof comprises SEQ ID NO: 105. In certain embodiments, the stapled peptide is of SEQ ID NO: 105, or a pharmaceutically acceptable salt thereof. In certain embodiments, the stapled is of SEQ ID NO: 105, or a pharmaceutically acceptable salt thereof, wherein the C-terminus is amidated with —NH₂. In certain embodiments, the stapled peptide is of SEQ ID NO: 105, or a pharmaceutically acceptable salt thereof, wherein the C-terminus of the peptide is amidated with —NH₂; and wherein X1 and X2, and X3 and X4, are each connected via the crosslink (alk).

In certain embodiments, the stapled peptide comprises the amino acid sequence:

(SEQ ID NO: 113)
G X¹ F S K X² K G K K I K N L L X³ S G L X⁴ K G
G E,

or a pharmaceutically acceptable salt thereof. In certain embodiments, the stapled peptide is of SEQ ID NO: 113, or a pharmaceutically acceptable salt thereof. In certain embodiments, the stapled peptide is of SEQ ID NO: 113, or a pharmaceutically acceptable salt thereof, wherein the C-terminus is amidated with —NH₂. In certain embodiments, the stapled peptide is of SEQ ID NO: 113, or a pharmaceutically acceptable salt thereof, wherein the C-terminus of the peptide is amidated with —NH₂; and wherein X¹ and X², and X³ and X⁴, are each connected via the crosslink (alk).

In some embodiments, the stapled peptide comprises one of the following amino acid sequences:

(SEQ ID NO: 115)
X¹ I F S X² L A G K K I K N L L I S G L K G,
(SEQ ID NO: 116)
G X¹ F S K X² A G K K I K N L L I S G L K G,
(SEQ ID NO: 117)
G I X¹ S K L X² G K K I K N L L I S G L K G,
(SEQ ID NO: 118)
G I F X¹ K L A X² K K I K N L L I S G L K G,
(SEQ ID NO: 119)
G I F S X¹ L A G X² K I K N L L I S G L K G,
(SEQ ID NO: 120)
G I F S K X¹ A G K X² I K N L L I S G L K G,
(SEQ ID NO: 121)
G I F S K L X¹ G K K X² K N L L I S G L K G,
(SEQ ID NO: 122)
G I F S K L A X¹ K K I X² N L L I S G L K G,
(SEQ ID NO: 123)
G I F S K L A G X¹ K I K X² L L I S G L K G,
(SEQ ID NO: 124)
G I F S K L A G K X¹ I K N X² L I S G L K G,
(SEQ ID NO: 125)
G I F S K L A G K K X¹ K N L X² I S G L K G,
(SEQ ID NO: 126)
G I F S K L A G K K I X¹ N L L X² S G L K G,
(SEQ ID NO: 127)
G I F S K L A G K K I K X¹ L L I X² G L K G,
(SEQ ID NO: 128)
G I F S K L A G K K I K N X¹ L I S X² L K G,
(SEQ ID NO: 129)
G I F S K L A G K K I K N L X¹ I S G X² K G,
(SEQ ID NO: 130)
G I F S K L A G K K I K N L L X¹ S G L X² G,
(SEQ ID NO: 131)
G I F S K L A G K K I K N L L I X¹ G L K X2,
(SEQ ID NO: 132)
X¹ I F S K L A X² K K I K N L L I S G L K G,
(SEQ ID NO: 133)
G X¹ F S K L A G X² K I K N L L I S G L K G,
(SEQ ID NO: 134)
G I X¹ S K L A G K X² I K N L L I S G L K G,
(SEQ ID NO: 135)
G I F X¹ K L A G K K X² K N L L I S G L K G,
(SEQ ID NO: 136)
G I F S X¹ L A G K K I X² N L L I S G L K G,
(SEQ ID NO: 137)
G I F S K X¹ A G K K I K X² L L I S G L K G,
(SEQ ID NO: 138)
G I F S K L A X¹ K K I K N L X² I S G L K G,
(SEQ ID NO: 139)
G I F S K L A G X¹ K I K N L L X² S G L K G,
(SEQ ID NO: 140)
G I F S K L A G K X¹ I K N L L I X² G L K G,
(SEQ ID NO: 141)
G I F S K L A G K K X¹ K N L L IS X² L K G,
(SEQ ID NO: 142)
G I F S K L A G K K I X¹ N L L I S G X² K G,
(SEQ ID NO: 143)
G I F S K L A G K K I K X¹ L L I S G L X² G,
(SEQ ID NO: 144)
G I F S K L A G K K I K N X¹ L I S G L K X2,
(SEQ ID NO: 145)
G I F S K L X¹ G K K I K N X² L I S G L K G,
(SEQ ID NO: 146)
G I F S K L X¹ G K K X² K N L L I X³ G L K X⁴,
(SEQ ID NO: 147)
G I F S K L X¹ G K K X² K N L L I S G L K G G G,

or a pharmaceutically acceptable salt thereof, wherein:
X¹ and X² are amino acids (i.e., crosslinked amino acids);
wherein the amino acid sequence includes 0 to 6 amino acid substitutions, inclusive, at positions other than X¹ and X². In certain embodiments, the amino acid sequence comprises 0 amino acid substitutions. In certain embodiments, the amino acid sequence comprises 1 amino acid substitution. In certain embodiments, the amino acid sequence comprises 2 amino acid substitutions. In certain embodiments, the amino acid sequence comprises 3 amino acid substitutions. In certain embodiments, the amino acid sequence comprises 4 amino acid substitutions. In certain embodiments, the amino acid sequence comprises 5 amino acid substitutions. In certain embodiments, the amino acid sequence comprises 6 amino acid substitutions. In certain embodiments, X¹ and X² are connected via a dithio crosslink (e.g., (mxy), (pxy), (but), (bbn), (bbf), (bbp), (pfb), (hfb)).

In certain embodiments, the stapled peptide is one of the following, or a pharmaceutically acceptable salt thereof:

| | | | | | | | | | | | | | | | | | | | | | | | | SEQ | X1—X2 | |
|---|---|---|---|---|---|---|---|---|---|---|---|---|---|---|---|---|---|---|---|---|---|---|---|---|---|---|
| StAMP # | Sequence | | | | | | | | | | | | | | | | | | | | | | | ID NO: | Crosslink | C-terminus |
| E2 | G | I | F | S | K | L | X¹ | G | K | K | X² | K | N | L | L | I | S | G | L | K | G | | | 121 | mxy | —NH₂ |
| E3 | G | I | F | S | K | L | X¹ | G | K | K | X² | K | N | L | L | I | S | G | L | K | G | | | 121 | pxy | —NH₂ |
| E4 | G | I | F | S | K | L | X¹ | G | K | K | X² | K | N | L | L | I | S | G | L | K | G | | | 121 | but | —NH₂ |
| E91 | G | I | F | S | K | L | X¹ | G | K | K | X² | K | N | L | L | I | S | G | L | K | G | | | 121 | mxy | —CO₂H |
| E92 | G | I | F | S | K | L | X¹ | G | K | K | X² | K | N | L | L | I | S | G | L | K | G | G | G | 147 | mxy | —CO₂H |

In certain embodiments, the stapled peptide is one of the following, or a pharmaceutically acceptable salt thereof:

| | | | | | | | | | | | | | | | | | | | | | | SEQ | X¹—X² | |
|---|---|---|---|---|---|---|---|---|---|---|---|---|---|---|---|---|---|---|---|---|---|---|---|---|
| StAMP # | Sequence | | | | | | | | | | | | | | | | | | | | | ID NO: | Crosslink | C-terminus |
| E1 | G | I | X¹ | S | K | L | X² | G | K | K | I | K | N | L | L | I | S | G | L | K | G | 117 | but | —NH₂ |
| E2 | G | I | F | S | K | L | X¹ | G | K | K | X² | K | N | L | L | I | S | G | L | K | G | 121 | mxy | —NH₂ |
| E3 | G | I | F | S | K | L | X¹ | G | K | K | X² | K | N | L | L | I | S | G | L | K | G | 121 | pxy | —NH₂ |
| E4 | G | I | F | S | K | L | X¹ | G | K | K | X² | K | N | L | L | I | S | G | L | K | G | 121 | but | —NH₂ |
| E5 | G | I | F | X¹ | K | L | A | X² | K | K | I | K | N | L | L | I | S | G | L | K | G | 118 | mxy | —NH₂ |
| E6 | G | I | F | X¹ | K | L | A | X² | K | K | I | K | N | L | L | I | S | G | L | K | G | 118 | pxy | —NH₂ |
| E7 | G | I | F | X¹ | K | L | A | X² | K | K | I | K | N | L | L | I | S | G | L | K | G | 118 | but | —NH₂ |
| E8 | G | I | F | S | K | X¹ | A | G | K | X² | I | K | N | L | L | I | S | G | L | K | G | 120 | pxy | —NH₂ |
| E9 | G | I | F | S | K | X¹ | A | G | K | X² | I | K | N | L | L | I | S | G | L | K | G | 120 | but | —NH₂ |
| E10 | G | I | F | S | K | L | A | X¹ | K | K | I | X² | N | L | L | I | S | G | L | K | G | 122 | mxy | —NH₂ |
| E11 | G | I | F | S | K | L | A | X¹ | K | K | I | X² | N | L | L | I | S | G | L | K | G | 122 | pxy | —NH₂ |
| E12 | G | I | F | S | K | L | A | X¹ | K | K | I | X² | N | L | L | I | S | G | L | K | G | 122 | but | —NH₂ |
| E13 | G | I | F | S | K | L | A | G | X¹ | K | I | K | X² | L | L | I | S | G | L | K | G | 123 | pxy | —NH₂ |
| E14 | G | I | F | S | K | L | A | G | K | X¹ | I | K | N | X² | L | I | S | G | L | K | G | 124 | mxy | —NH₂ |
| E15 | G | I | F | S | K | L | A | G | K | X¹ | I | K | N | X² | L | I | S | G | L | K | G | 124 | pxy | —NH₂ |
| E16 | G | I | F | S | K | L | A | G | K | X¹ | I | K | N | X² | L | I | S | G | L | K | G | 124 | but | —NH₂ |
| E17 | G | I | F | S | K | L | A | G | K | K | X¹ | K | N | L | X² | I | S | G | L | K | G | 125 | mxy | —NH₂ |
| E18 | G | I | F | S | K | L | A | G | K | K | X¹ | K | N | L | X² | I | S | G | L | K | G | 125 | pxy | —NH₂ |
| E19 | G | I | F | S | K | L | A | G | K | K | X¹ | K | N | L | X² | I | S | G | L | K | G | 125 | but | —NH₂ |
| E20 | G | I | F | S | K | L | A | G | K | K | I | X¹ | N | L | L | X² | S | G | L | K | G | 126 | mxy | —NH₂ |
| E21 | G | I | F | S | K | L | A | G | K | K | I | X¹ | N | L | L | X² | S | G | L | K | G | 126 | pxy | —NH₂ |
| E22 | G | I | F | S | K | L | A | G | K | K | I | X¹ | N | L | L | X² | S | G | L | K | G | 126 | but | —NH₂ |
| E23 | G | I | F | S | K | L | A | G | K | K | I | K | X¹ | L | L | I | X² | G | L | K | G | 127 | mxy | —NH₂ |
| E24 | G | I | F | S | K | L | A | G | K | K | I | K | X¹ | L | L | I | X² | G | L | K | G | 127 | pxy | —NH₂ |
| E25 | G | I | F | S | K | L | A | G | K | K | I | K | X¹ | L | L | I | X² | G | L | K | G | 127 | but | —NH₂ |
| E26 | G | I | F | S | K | L | A | G | K | K | I | K | N | X¹ | L | I | S | X² | L | K | G | 128 | mxy | —NH₂ |
| E27 | G | I | F | S | K | L | A | G | K | K | I | K | N | X¹ | L | I | S | X² | L | K | G | 128 | pxy | —NH₂ |
| E28 | G | I | F | S | K | L | A | G | K | K | I | K | N | X¹ | L | I | S | X² | L | K | G | 128 | but | —NH₂ |
| E29 | G | I | F | S | K | L | A | G | K | K | I | K | N | L | X¹ | I | S | G | X² | K | G | 129 | mxy | —NH₂ |
| E30 | G | I | F | S | K | L | A | G | K | K | I | K | N | L | X¹ | I | S | G | X² | K | G | 129 | pxy | —NH₂ |
| E31 | G | I | F | S | K | L | A | G | K | K | I | K | N | L | X¹ | I | S | G | X² | K | G | 129 | but | —NH₂ |
| E32 | G | I | F | S | K | L | A | G | K | K | I | K | N | L | L | X¹ | S | G | L | X² | G | 130 | mxy | —NH₂ |
| E33 | G | I | F | S | K | L | A | G | K | K | I | K | N | L | L | X¹ | S | G | L | X² | G | 130 | pxy | —NH₂ |
| E34 | G | I | F | S | K | L | A | G | K | K | I | K | N | L | L | X¹ | S | G | L | X² | G | 130 | but | —NH₂ |
| E35 | G | I | F | S | K | L | A | G | K | K | I | K | N | L | L | I | X¹ | G | L | K | X² | 131 | but | —NH₂ |
| E36 | X¹ | I | F | S | X² | L | A | G | K | K | I | K | N | L | L | I | S | G | L | K | G | 115 | mxy | —NH₂ |
| E37 | X¹ | I | F | S | X² | L | A | G | K | K | I | K | N | L | L | I | S | G | L | K | G | 115 | pxy | —NH₂ |
| E38 | X¹ | I | F | S | X² | L | A | G | K | K | I | K | N | L | L | I | S | G | L | K | G | 115 | but | —NH₂ |
| E39 | G | I | X¹ | S | K | L | X² | G | K | K | I | K | N | L | L | I | S | G | L | K | G | 117 | mxy | —NH₂ |
| E40 | G | I | X¹ | S | K | L | X² | G | K | K | I | K | N | L | L | I | S | G | L | K | G | 117 | pxy | —NH₂ |
| E41 | G | I | F | S | K | X¹ | A | G | K | X² | I | K | N | L | L | I | S | G | L | K | G | 120 | mxy | —NH₂ |
| E42 | G | I | F | S | K | L | A | G | X¹ | K | I | K | X² | L | L | I | S | G | L | K | G | 123 | mxy | —NH₂ |
| E43 | G | I | F | S | K | L | A | G | X¹ | K | I | K | X² | L | L | I | S | G | L | K | G | 123 | but | —NH₂ |
| E44 | G | I | F | S | K | L | A | G | K | K | I | K | N | L | L | I | X¹ | G | L | K | X² | 131 | mxy | —NH₂ |
| E45 | G | I | F | S | K | L | A | G | K | K | I | K | N | L | L | I | X¹ | G | L | K | X² | 131 | pxy | —NH₂ |
| E46 | G | X¹ | F | S | K | X² | A | G | K | K | I | K | N | L | L | I | S | G | L | K | G | 116 | mxy | —NH₂ |
| E47 | G | X¹ | F | S | K | X² | A | G | K | K | I | K | N | L | L | I | S | G | L | K | G | 116 | pxy | —NH₂ |
| E48 | G | X¹ | F | S | K | X² | A | G | K | K | I | K | N | L | L | I | S | G | L | K | G | 116 | but | —NH₂ |

In certain embodiments, the stapled peptide is one of the following, or a pharmaceutically acceptable salt thereof:

| | | | | | | | | | | | | | | | | | | | | | | SEQ | X¹—X² | |
|---|---|---|---|---|---|---|---|---|---|---|---|---|---|---|---|---|---|---|---|---|---|---|---|---|
| StAMP # | Sequence | | | | | | | | | | | | | | | | | | | | | ID NO: | Crosslink | C-terminus |
| E49 | X¹ | I | F | S | K | L | A | X² | K | K | I | K | N | L | L | I | S | G | L | K | G | 132 | bbp | —NH₂ |
| E50 | X¹ | I | F | S | K | L | A | X² | K | K | I | K | N | L | L | I | S | G | L | K | G | 132 | bbn | —NH₂ |
| E51 | X¹ | I | F | S | K | L | A | X² | K | K | I | K | N | L | L | I | S | G | L | K | G | 132 | bbf | —NH₂ |
| E52 | G | X¹ | F | S | K | L | A | G | X² | K | I | K | N | L | L | I | S | G | L | K | G | 133 | bbn | —NH₂ |
| E53 | G | X¹ | F | S | K | L | A | G | X² | K | I | K | N | L | L | I | S | G | L | K | G | 133 | bbf | —NH₂ |
| E54 | G | I | X¹ | S | K | L | A | G | K | X² | I | K | N | L | L | I | S | G | L | K | G | 134 | bbp | —NH₂ |
| E55 | G | I | X¹ | S | K | L | A | G | K | X² | I | K | N | L | L | I | S | G | L | K | G | 134 | bbn | —NH₂ |
| E56 | G | I | X¹ | S | K | L | A | G | K | X² | I | K | N | L | L | I | S | G | L | K | G | 134 | bbf | —NH₂ |
| E57 | G | I | F | X¹ | K | L | A | G | K | K | X² | K | N | L | L | I | S | G | L | K | G | 135 | bbp | —NH₂ |
| E58 | G | I | F | X¹ | K | L | A | G | K | K | X² | K | N | L | L | I | S | G | L | K | G | 135 | bbn | —NH₂ |
| E59 | G | I | F | X¹ | K | L | A | G | K | K | X² | K | N | L | L | I | S | G | L | K | G | 135 | bbf | —NH₂ |
| E60 | G | I | F | S | X¹ | L | A | G | K | K | I | X² | N | L | L | I | S | G | L | K | G | 136 | bbp | —NH₂ |
| E61 | G | I | F | S | X¹ | L | A | G | K | K | I | X² | N | L | L | I | S | G | L | K | G | 136 | bbf | —NH₂ |
| E62 | G | I | F | S | X¹ | L | A | G | K | K | I | X² | N | L | L | I | S | G | L | K | G | 136 | bbn | —NH₂ |
| E63 | G | I | F | S | K | X¹ | A | G | K | K | I | K | X² | L | L | I | S | G | L | K | G | 137 | bbp | —NH₂ |
| E64 | G | I | F | S | K | X¹ | A | G | K | K | I | K | X² | L | L | I | S | G | L | K | G | 137 | bbn | —NH₂ |
| E65 | G | I | F | S | K | X¹ | A | G | K | K | I | K | X² | L | L | I | S | G | L | K | G | 137 | bbf | —NH₂ |
| E66 | G | I | F | S | K | L | A | X¹ | K | K | I | K | N | L | X² | I | S | G | L | K | G | 138 | bbp | —NH₂ |
| E67 | G | I | F | S | K | L | A | X¹ | K | K | I | K | N | L | X² | I | S | G | L | K | G | 138 | bbn | —NH₂ |
| E68 | G | I | F | S | K | L | A | X¹ | K | K | I | K | N | L | X² | I | S | G | L | K | G | 138 | bbf | —NH₂ |
| E69 | G | I | F | S | K | L | A | G | X¹ | K | I | K | N | L | L | X² | S | G | L | K | G | 139 | bbn | —NH₂ |
| E70 | G | I | F | S | K | L | A | G | X¹ | K | I | K | N | L | L | X² | S | G | L | K | G | 139 | bbf | —NH₂ |
| E71 | G | I | F | S | K | L | A | G | X¹ | K | I | K | N | L | L | X² | S | G | L | K | G | 139 | bbp | —NH₂ |
| E72 | G | I | F | S | K | L | A | G | K | X¹ | I | K | N | L | L | I | X² | G | L | K | G | 140 | bbp | —NH₂ |
| E73 | G | I | F | S | K | L | A | G | K | X¹ | I | K | N | L | L | I | X² | G | L | K | G | 140 | bbn | —NH₂ |
| E74 | G | I | F | S | K | L | A | G | K | X¹ | I | K | N | L | L | I | X² | G | L | K | G | 140 | bbf | —NH₂ |
| E75 | G | I | F | S | K | L | A | G | K | K | X¹ | K | N | L | L | I | S | X² | L | K | G | 141 | bbn | —NH₂ |
| E76 | G | I | F | S | K | L | A | G | K | K | X¹ | K | N | L | L | I | S | X² | L | K | G | 141 | bbf | —NH₂ |
| E77 | G | I | F | S | K | L | A | G | K | K | X¹ | K | N | L | L | I | S | X² | L | K | G | 141 | bbp | —NH₂ |
| E78 | G | I | F | S | K | L | A | G | K | K | I | X¹ | N | L | L | I | S | G | X² | K | G | 142 | bbn | —NH₂ |
| E79 | G | I | F | S | K | L | A | G | K | K | I | X¹ | N | L | L | I | S | G | X² | K | G | 142 | bbf | —NH₂ |
| E80 | G | I | F | S | K | L | A | G | K | K | I | C | N | L | L | I | S | G | C | K | G | 142 | bbp | —NH₂ |
| E81 | G | I | F | S | K | L | A | G | K | K | I | K | X¹ | L | L | I | S | G | L | X² | G | 143 | bbp | —NH₂ |
| E82 | G | I | F | S | K | L | A | G | K | K | I | K | X¹ | L | L | I | S | G | L | X² | G | 143 | bbn | —NH₂ |
| E83 | G | I | F | S | K | L | A | G | K | K | I | K | X¹ | L | L | I | S | G | L | X² | G | 143 | bbf | —NH₂ |
| E84 | G | I | F | S | K | L | A | G | K | K | I | K | N | X¹ | L | I | S | G | L | K | X² | 144 | bbp | —NH₂ |
| E85 | G | I | F | S | K | L | A | G | K | K | I | K | N | X¹ | L | I | S | G | L | K | X² | 144 | bbn | —NH₂ |
| E86 | G | I | F | S | K | L | A | G | K | K | I | K | N | X¹ | L | I | S | G | L | K | X² | 144 | bbf | —NH₂ |
| E87 | G | I | F | S | K | L | X¹ | G | K | K | I | K | N | X² | L | I | S | G | L | K | G | 145 | bbn | —NH₂ |
| E88 | G | I | F | S | K | L | X¹ | G | K | K | I | K | N | X² | L | I | S | G | L | K | G | 145 | bbf | —NH₂ |
| E89 | G | I | F | S | K | L | X¹ | G | K | K | I | K | N | X² | L | I | S | G | L | K | G | 145 | bbp | —NH₂ |

In certain embodiments, the stapled peptide is the following, or a pharmaceutically acceptable salt thereof:

| | | | | | | | | | | | | | | | | | | | | | | | X¹—X² and | |
|---|---|---|---|---|---|---|---|---|---|---|---|---|---|---|---|---|---|---|---|---|---|---|---|---|
| | | | | | | | | | | | | | | | | | | | | | | SEQ | X³—X⁴ | |
| StAMP # | Sequence | | | | | | | | | | | | | | | | | | | | | ID NO: | Crosslink | C-terminus |
| E90 | G | I | F | S | K | L | X¹ | G | K | K | X² | K | N | L | L | I | X³ | G | L | K | X⁴ | 146 | pxy | —NH₂ |

### Stapled Peptide Crosslinks

As described herein, stapled peptides comprise crosslinks (e.g., staples), wherein each crosslink connects two amino acids (i.e., crosslinked amino acids) to form a macrocycle. In certain embodiments, when an amino acid sequence (e.g., SEQ ID NOs: 1-147 provided herein) comprises X¹ and X², X¹ and X² are crosslinked amino acids connected via a crosslink. Likewise, in certain embodiments, when an amino acid sequence (e.g., SEQ ID NOs: 1-147 provided herein) comprises X³ and X⁴, X³ and X⁴ are crosslinked amino acids connected via a crosslink. The following certain embodiments describing stapled peptide crosslinks apply to all stapled peptides described herein, including all amino acid sequences (e.g., SEQ ID NOs: 1-147) and variants thereof described herein.

In certain embodiments, the crosslinks are independently attached to the α-positions of the crosslinked amino acids (e.g., α-positions of X¹, X², X³, and X⁴). In certain embodiments, the crosslinks are independently attached to the α-positions of the crosslinked amino acids (e.g., X¹, X², X³, and X⁴), and the crosslinked amino acids are independently α,α-disubstituted amino acids.

In certain embodiments, each crosslink is independently from about 5 Å to about 35 Å in length, inclusive. In certain embodiments, each crosslink is independently from about 5 Å to about 25 Å in length, inclusive (e.g., in the case of i+4 crosslinks). In certain embodiments, each crosslink is independently from about 6 Å to about 22 Å in length, inclusive. In certain embodiments, each crosslink is independently from about 7 Å to about 20 Å in length, inclusive. In certain embodiments, each crosslink is independently from about 8 Å to about 18 Å in length, inclusive. In certain embodiments, each crosslink is independently from about 9 Å to about 17 Ain length, inclusive. each crosslink is independently about 10 Å to about 16 Å in length, inclusive. In certain embodiments, each crosslink is independently from about 11 Å to about 15 Å in length, inclusive. In certain embodiments, each crosslink is independently from about 12 Å to about 14 Å in length, inclusive. In certain embodiments, each crosslink is independently about 5, 6, 7, 8, 9, 10, 11, 12, 13, 14, 15, 16, 17, 18, 19, 20, 21, 22, 23, 24, or 25 Å in length.

In certain embodiments, each crosslink is independently from about 15 Å to about 35 Å in length, inclusive (e.g., in the case of i+7 crosslinks). In certain embodiments, each crosslink is independently from about 17 Å to about 33 Å in length, inclusive. In certain embodiments, each crosslink is independently from about 19 Å to about 31 Å in length, inclusive. In certain embodiments, each crosslink is independently from about 20 Å to about 30 Å in length, inclusive. In certain embodiments, each crosslink is independently from about 22 Å to about 29 Ain length, inclusive. each crosslink is independently about 24 Å to about 28 Å in length, inclusive. In certain embodiments, each crosslink is independently from about 25 Å to about 27 Ain length, inclusive. In certain embodiments, each crosslink is independently about 15, 16, 17, 18, 19, 20, 21, 22, 23, 24, 25, 26, 27, 28, 29, 30, 31, 32, 33, 34, or 35 Ain length.

In certain embodiments, the length of each crosslink is approximately equal to the length of 5 to 25 carbon-carbon and/or carbon-sulfur bonds, inclusive. In certain embodiments, the length of each crosslink is approximately equal to the length of 5, 6, 7, 8, 9, 10, 11, 12, 13, 14, 15, 16, 17, 18, 19, 20, 21, 22, 23, 24, or 25 carbon-carbon and/or carbon-sulfur bonds, inclusive.

In certain embodiments, the length of each crosslink is approximately equal to the length of 5 to 20 carbon-carbon and/or carbon-sulfur bonds, inclusive. In certain embodiments, the length of each crosslink is approximately equal to the length of 5 to 15 carbon-carbon and/or carbon-sulfur bonds, inclusive. In certain embodiments, the length of each crosslink is approximately equal to the length of 5 to 13 carbon-carbon and/or carbon-sulfur bonds, inclusive. In certain embodiments, the length of each crosslink is approximately equal to the length of 6 to 12 carbon-carbon and/or carbon-sulfur bonds, inclusive. In certain embodiments, the length of each crosslink is approximately equal to the length of 7 to 11 carbon-carbon and/or carbon-sulfur bonds, inclusive. In certain embodiments, the length of each crosslink is approximately equal to the length of 8 to 10 carbon-carbon and/or carbon-sulfur bonds, inclusive.

In certain embodiments, the length of each crosslink is approximately equal to the length of 10 to 20 carbon-carbon and/or carbon-sulfur bonds, inclusive. In certain embodiments, the length of each crosslink is approximately equal to the length of 11 to 19 carbon-carbon and/or carbon-sulfur bonds, inclusive. In certain embodiments, the length of each crosslink is approximately equal to the length of 12 to 18 carbon-carbon and/or carbon-sulfur bonds, inclusive. In certain embodiments, the length of each crosslink is approximately equal to the length of 13 to 17 carbon-carbon and/or carbon-sulfur bonds, inclusive. In certain embodiments, the length of each crosslink is approximately equal to the length of 14 to 16 carbon-carbon and/or carbon-sulfur bonds, inclusive.

In certain embodiments, at least one crosslink spans at least one turn of an α-helix of the peptide. In certain embodiments, each crosslink spans at least one turn of an α-helix of the peptide. In certain embodiments, at least one crosslink spans one turn of an α-helix of the peptide. In certain embodiments, each crosslink spans one turn of an α-helix of the peptide.

In certain embodiments, each pair of crosslinked amino acids (e.g., X¹ and X², and X³ and X⁴) are independently connected by a crosslink to form the following formula:

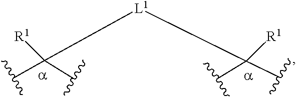

wherein α denotes the α-carbons of the crosslinked amino acids; L¹ is a crosslink; and each instance of R¹ is independently hydrogen or optionally substituted C₁₋₆ alkyl.

In certain embodiments, each crosslink (e.g., L¹) is independently optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof.

In certain embodiments, each crosslink (e.g., L¹) is independently a hydrocarbon crosslink. “Hydrocarbon crosslink” for the purposes of this disclosure is a crosslink consisting of optionally substituted alkylene, optionally substituted alkenylene, optionally substituted alkynylene, and combinations thereof.

In certain embodiments, each crosslink (e.g., L¹) is independently optionally substituted alkenylene (e.g., unsubstituted alkenylene). In certain embodiments, each crosslink is independently of the following formula:

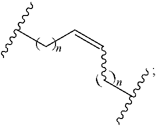

wherein each n is independently an integer from 1-10, inclusive. In certain embodiments, the sum of two n on the same crosslink is 6.

In certain embodiments, the crosslinked amino acids (e.g., X¹, X², X³, and X⁴) are independently α,α-disubstituted amino acids. For instance, in certain embodiments, each pair of crosslinked amino acids (e.g., X¹ and X², and X³ and X⁴) are independently connected by a crosslink to form the following formula:

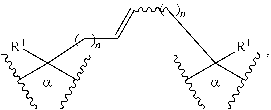

wherein α denotes the α-carbons of the crosslinked amino acids; and wherein each instance of R¹ is independently optionally substituted C₁₋₆ alkyl. In certain embodiments, the sum of two n on the same crosslink is 6.

For example, in certain embodiments, a crosslink (e.g., L¹) is independently of the formula:

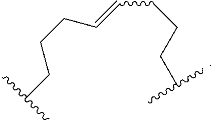 [SMILES: *CCCC=CCCC*]

For example, in certain embodiments, a pair of crosslinked amino acids (e.g., X¹ and X², and X³ and X⁴) are independently connected via a crosslink to form the following formula:

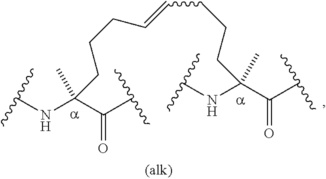 [SMILES: *N[C@@](C)(CCCC=CCCC[C@](C)(N*)C(*)=O)C(*)=O]

wherein α denotes the α-carbons of the crosslinked amino acids.

In certain embodiments, X¹ and X² are connected to form the formula (alk).

In certain embodiments, X³ and X⁴ are connected to form the formula (alk).

In certain embodiments, a crosslink (e.g., L¹) is independently optionally substituted alkylene (e.g., unsubstituted alkylene). In certain embodiments, each crosslink is independently of the following formula:

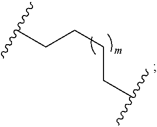

wherein m is an integer from 1-20, inclusive. In certain embodiments, m is 6.

In certain embodiments, a pair of crosslinked amino acids (e.g., X¹ and X², and X³ and X⁴) are independently joined by a crosslink to form the following formula:

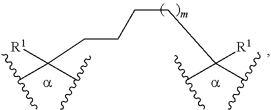

wherein α denotes the α-carbons of the crosslinked amino acids; and wherein each instance of R¹ is independently optionally substituted C₁₋₆ alkyl. In certain embodiments, m is 6.

For example, in certain embodiments, a crosslink (e.g., L¹) is independently of the formula:

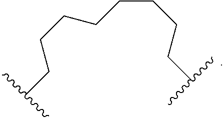 [SMILES: *CCCCCCCC*]

For example, in certain embodiments, a pair of crosslinked amino acids (e.g., X¹ and X², and X³ and X⁴) are connected via a crosslink to form the following formula:

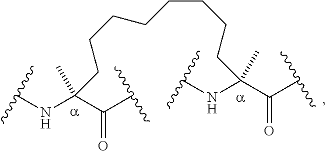 [SMILES: *N[C@@](C)(CCCCCCCC[C@](C)(N*)C(*)=O)C(*)=O]

wherein α denotes the α-carbons of the crosslinked amino acids.

In certain embodiments, a crosslink (e.g., L¹) is independently of the formula:

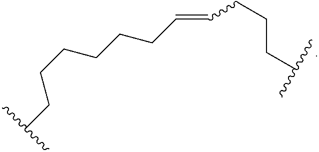 [SMILES: *CCCC=CCCCCCC*]

For example, in certain embodiments, a pair of crosslinked amino acids (e.g., X¹ and X², and X³ and X⁴) are independently connected via a crosslink to form the following formula:

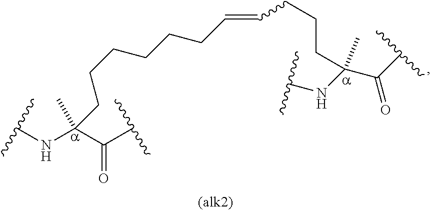 [SMILES: *N[C@@](C)(CCCC=CCCCCCC[C@](C)(N*)C(*)=O)C(*)=O]

wherein α denotes the α-carbons of the crosslinked amino acids.

In certain embodiments, a crosslink (e.g., L¹) is independently of the formula:

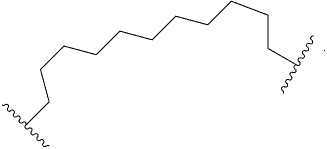 [SMILES: *CCCCCCCCCCC*]

For example, in certain embodiments, a pair of crosslinked amino acids (e.g., X¹ and X², and X³ and X⁴) are independently connected via a crosslink to form the following formula:

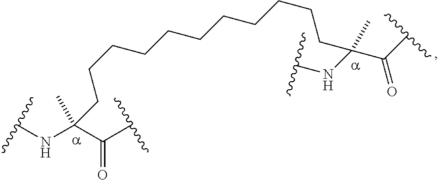 [SMILES: *N[C@@](C)(CCCCCCCCCCC[C@](C)(N*)C(*)=O)C(*)=O]

wherein α denotes the α-carbons of the crosslinked amino acids.

In certain embodiments, a crosslink (e.g., L¹) is independently optionally substituted alkynylene (e.g., unsubstituted alkynylene).

In certain embodiments, a crosslink (e.g., L¹) is independently a dithio crosslink. For the purposes of this disclosure, a “dithio crosslink” (i.e., “dithio staple”) is a crosslink comprising two thioethers (i.e., two —S— groups). In certain embodiments, a crosslink is independently a dithio crosslink of the following formula:

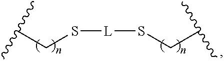

wherein each n is independently an integer from 1-10, inclusive; and

L is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof. In certain embodiments, each instance of n is 1. In certain embodiments, each instance of n is 2.

In certain embodiments, a crosslink is independently a dithio crosslink of the following formula:

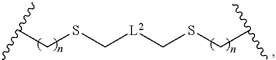

wherein each n is independently an integer from 1-10, inclusive; and

L² is optionally substituted alkylene,

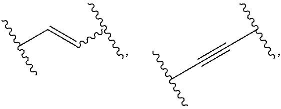

optionally substituted arylene, optionally substituted heteroarylene, or -A¹-A¹-; wherein each instance of A¹ is independently optionally substituted arylene or optionally substituted heteroarylene. In certain embodiments, each instance of n is 1. In certain embodiments, each instance of n is 2.

In certain embodiments, a crosslink is independently a dithio crosslink of one of the following formulae:

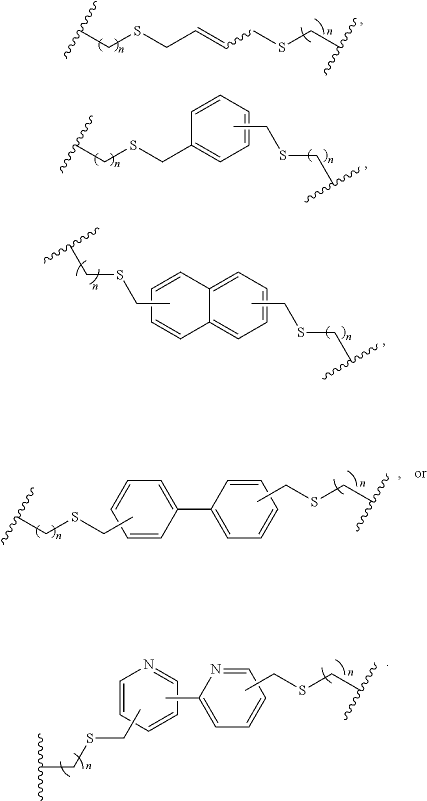

In certain embodiments, each instance of n is 1. In certain embodiments, each instance of n is 2.

For example, in certain embodiments, a crosslink is independently a dithio crosslink of one of the following formulae:

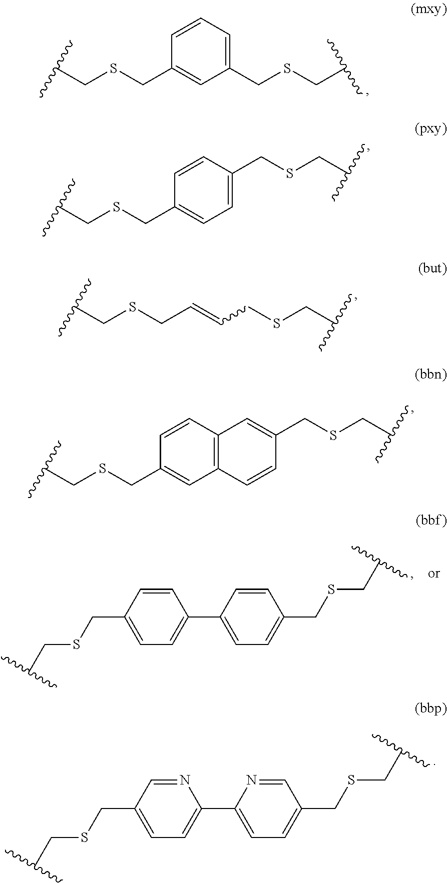 [SMILES: *CSCc1cccc(CSC*)c1 ; *CSCc1ccc(CSC*)cc1 ; *CSCC=CCSC* ; *CSCc1ccc2cc(CSC*)ccc2c1 ; *CSCc1ccc(-c2ccc(CSC*)cc2)cc1 ; *CSCc1ccc(-c2ccc(CSC*)cn2)nc1]

In other embodiments, a crosslink is independently a dithio crosslink of the following formula:

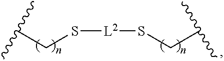

wherein each n is independently an integer from 1-10, inclusive;

L² is an optionally substituted aromatic ring (e.g., a polyhalogenated aryl or heteroaryl ring) or -A¹-A¹-; wherein each instance of A¹ is independently an optionally substituted aromatic ring (e.g., a polyhalogenated aryl or heteroaryl ring). In certain embodiments, each instance of n is 1. In certain embodiments, each instance of n is 2.

In certain embodiments, a crosslink is independently a dithio crosslink of one of the following formulae:

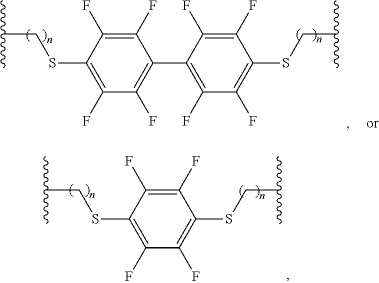

Wherein each n is independently an integer from 1-10, inclusive. In certain embodiments, each instance of n is 1. In certain embodiments, each instance of n is 2.

For example, in certain embodiments, a crosslink is independently of one of the following formulae:

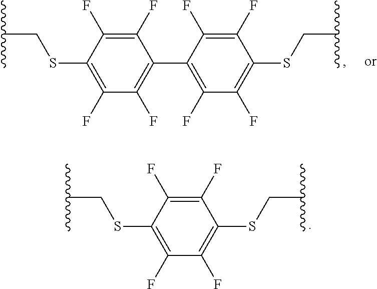 [SMILES: *CSc1c(F)c(F)c(-c2c(F)c(F)c(SC*)c(F)c2F)c(F)c1F ; *CSc1c(F)c(F)c(SC*)c(F)c1F]

For the purposes of this disclosure, the particular crosslinks (mxy), (pxy), (but), (bbn), (bbf), (bbp), (pfb), and (hfb) referenced herein are formed by crosslinking two cysteine (C) residues of a peptide. In other words, a peptide or pharmaceutically acceptable salt thereof provided herein comprising a (mxy), (pxy), (but), (bbn), (bbf), (bbp), (pfb), and/or (hfb) crosslink includes each pair of crosslinked amino acids (e.g., X¹ and X², X³ and X⁴) connected via a dithio crosslink to form the following formula:

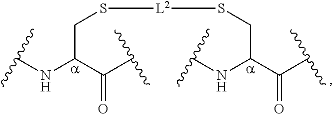

wherein each α represents the alpha-position of a dithio-crosslinked amino acid (e.g., X¹, X², X³, X⁴), and L² is as indicated in Table 4 below.

TABLE 4
Certain Dithio Crosslinks

| Dithio Crosslink | L² |
|---|---|
| mxy | 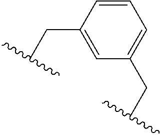 [SMILES: *Cc1cccc(C*)c1] |
| pxy | 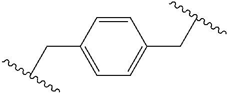 [SMILES: *Cc1ccc(C*)cc1] |
| but | 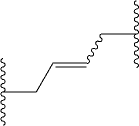 [SMILES: *CC=CC*] |
| bbn | 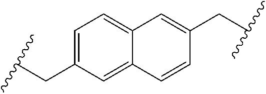 [SMILES: *Cc1ccc2cc(C*)ccc2c1] |
| bbf | 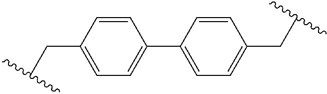 [SMILES: *Cc1ccc(-c2ccc(C*)cc2)cc1] |
| bbp | 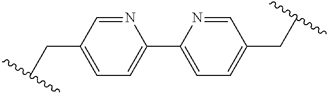 [SMILES: *Cc1ccc(-c2ccc(C*)cn2)nc1] |
| pfb | 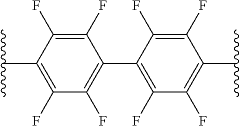 [SMILES: *c1c(F)c(F)c(-c2c(F)c(F)c(*)c(F)c2F)c(F)c1F] |
| hfb | 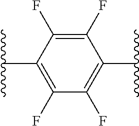 [SMILES: *c1c(F)c(F)c(*)c(F)c1F] |

In certain embodiments, a crosslink (e.g., L¹) is independently a triazolylene crosslink. For the purpose of this disclosure, a “triazolylene crosslink” is a crosslink interrupted by at least one triazolylene moiety (e.g.,

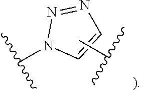

In certain embodiments, a crosslink is independently a triazolylene crosslink of the following formula:

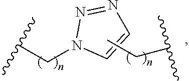

wherein each n is independently an integer from 1-10, inclusive. In certain embodiments, the sum of two n on the same crosslink is 5.

For example, in certain embodiments, a crosslink is independently a triazolylene crosslink of one of the following formulae:

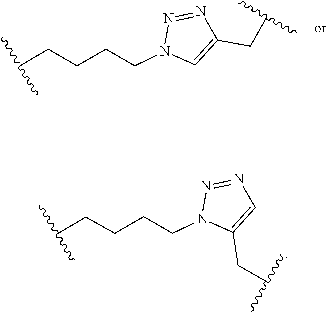 [SMILES: *CCCCn1cc(C*)nn1 ; *CCCCn1nncc1C*]

The following embodiments for n and R¹ apply to all generic formulae and subgenera provided herein, as well as all stapled and unstapled peptides provided herein.

In certain embodiments, the sum of two n on the same crosslink is an integer from 3-9, inclusive. In certain embodiments, the sum of two n on the same crosslink is an integer from 4-8, inclusive. In certain embodiments, the sum of two n on the same crosslink is an integer from 5-7, inclusive. In certain embodiments, the sum of two n on the same crosslink is 5. In certain embodiments, the sum of two n on the same crosslink is 6. In certain embodiments, the sum of two n on the same crosslink is 7.

In certain embodiments, at least one instance of n is 1. In certain embodiments, at least one instance of n is 2. In certain embodiments, at least one instance of n is 3. In certain embodiments, at least one instance of n is 4. In certain embodiments, at least one instance of n is 5. In certain embodiments, at least one instance of n is 6. In certain embodiments, at least one instance of n is 7. In certain embodiments, at least one instance of n is 8. In certain embodiments, at least one instance of n is 9. In certain embodiments, at least one instance of n is 10.

In certain embodiments, m is an integer from 3-9, inclusive. In certain embodiments, m is an integer from 4-8, inclusive. In certain embodiments, m is an integer from 5-7, inclusive. In certain embodiments, m is 5. In certain embodiments, m is 6. In certain embodiments, m is 7.

In certain embodiments, at least one instance of R¹ is hydrogen. In certain embodiments, each instance of R¹ is hydrogen. In certain embodiments, at least one instance of R¹ is unsubstituted C₁₋₆ alkyl. In certain embodiments, at least one instance of R¹ is unsubstituted C₁₋₃ alkyl. In certain embodiments, at least one instance of R¹ is methyl. In certain embodiments, each instance of R¹ is methyl.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### Peptide C-Terminus Modifications

Stapled peptides can comprise one or more additional modifications anywhere on the peptide (e.g., on an amino acid sidechain, on an α-carbon of an amino acid, on a peptidic nitrogen, at the C-terminus, N-terminus, etc.). Stapled peptides can comprise modifications to the C-terminus and/or N-terminus of the polypeptide. In certain embodiments, a stapled peptide comprises a modified C-terminus. Examples of C-terminus modifications are described herein.

In certain embodiments, the stapled peptide comprises an amidated C-terminus. Traditionally, peptides comprise a carboxyl group (—C(═O)OH) at the C-terminus. The stapled peptide may comprise an amide at the C-terminus (e.g., —C(═O)NR₂, wherein the group NR₂ is NH₂, monosubstituted amino, disubstituted amino, or trisubstituted amino), referred to as “amidated C-terminus.” For example, a peptide with a “C-terminus amidated with —NH₂” comprises the group —C(═O)NH₂ at the C-terminus instead of carboxyl (—C(═O)OH). An amidated C-terminus can also be represented by including —NR₂ (e.g., —NH₂) at the end of an amino acid sequence.

Stapled peptides may also be amidated at the C-terminus with an amino acid, peptide, or protein. The amino acid, peptide, or protein can be natural or unnatural. In certain embodiments, the stapled peptide comprises a peptide conjugated to the C-terminus. In certain embodiments, the peptide is from 2 to 6 amino acids in length, inclusive, and comprises amino acids selected from G, E, S, A, and K.

In certain embodiments, the peptide is from 2 to 6 amino acids in length, inclusive, and comprises amino acids selected from G, E, and S. In certain embodiments, the peptide is from 2 to 6 amino acids in length, inclusive, and comprises amino acids selected from G and E. In certain embodiments, the peptide is 2 amino acids in length and comprises amino acids selected from G and E. In certain embodiments, the peptide is 3 amino acids in length and comprises amino acids selected from G and E. In certain embodiments, the peptide is 4 amino acids in length and comprises amino acids selected from G and E. Non-limited examples of peptides which can be conjugated to the C-terminus of the stapled peptide are the following:

GE,
AG,
AA,
GG,
GGE,
GGS,
GGG,
GGK,
GGQ,
(SEQ ID NO: 187)
GGGC,
(SEQ ID NO: 156)
GGGE,
(SEQ ID NO: 157)
GGEE,
or
(SEQ ID NO: 158)
GGSGGS.

Stapled peptides may also comprise a small molecule, lipophilic group, or polymer conjugated to the C-terminus of the peptide.

In certain embodiments, the stapled peptide comprises a lipophilic group conjugated to the C-terminus of the peptide. In certain embodiments, the lipophilic group is a lipid or fatty acid. In certain embodiments, the lipophilic group is a hydrocarbon chain.

In certain embodiments, the stapled peptide comprises a polymer conjugated to the C-terminus of the peptide. In certain embodiments, the polymer is a polyether, e.g., polyethylene glycol (PEG). In certain embodiments, the polymer is PEG. In certain embodiments, the polymer is PEG3. As described herein, PEG3 is of the formula:

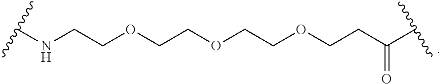 [SMILES: *NCCOCCOCCOCCC(*)=O ; "."]

In certain embodiments, the stapled peptide is amidated at the C-terminus with a group of the following formula: —NH—(PEG)-CONH₂, wherein PEG is polyethylene glycol. In certain embodiments, the stapled peptide is amidated at the C-terminus with a group of the following formula: —NH(CH₂CH₂O)₁₋₂₀CH₂CH₂CONH₂. In certain embodiments, the stapled peptide is amidated at the C-terminus with a group of one of the following formulae: —NHCH₂CH₂OCH₂CH₂CONH₂, —NH(CH₂CH₂O)₂—CH₂CH₂CONH₂, —NH(CH₂CH₂O)₃—CH₂CH₂CONH₂, —NH(CH₂CH₂O)₄—CH₂CH₂CONH₂, or —NH(CH₂CH₂O)₅—CH₂CH₂CONH₂.

In certain embodiments, the stapled peptide comprises a small molecule conjugated to the C-terminus of the peptide. In certain embodiments, the small molecule is an anti-cancer agent.

### Linker and Conjugation

As described herein, a SPAC provided herein comprises a stapled peptide conjugated to an antibody or antigen-binding fragment thereof. In certain embodiments, the antibody or antigen-binding fragment thereof is conjugated to the N-terminus of the stapled peptide. In certain embodiments, the antibody or antigen-binding fragment thereof is conjugated to the C-terminus of the stapled peptide. In certain embodiments, the antibody or antigen-binding fragment thereof is conjugated to an internal position on the stapled peptide (e.g., to an amino acid residue or to the crosslink of the stapled peptide).

In certain embodiments, the stapled peptide is conjugated through a thiol of the antibody or antigen-binding fragment thereof. In certain embodiments, the stapled peptide is conjugated through cysteine residue of the antibody or antigen-binding fragment thereof. In certain embodiments, the stapled peptide is conjugated through an amine of the antibody or antigen-binding fragment thereof. In certain embodiments, the stapled peptide is conjugated through a lysine residue of the antibody or antigen-binding fragment thereof.

In certain embodiments, the antibody or antigen-binding fragment thereof is conjugated to the stapled peptide directly (e.g., via a bond). In certain embodiments, the antibody or antigen-binding fragment thereof is conjugated to the stapled peptide via a linker. “Linker,” as used herein, refers to the moiety linking the antibody or antigen-binding fragment thereof to the stapled peptide, not to be confused with the one or more “crosslinks” connecting amino acids of the stapled peptides.

In certain embodiments, the linker comprises optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof.

In certain embodiments, the linker comprises optionally substituted alkylene. In certain embodiments, the linker comprises optionally substituted alkenylene. In certain embodiments, the linker comprises optionally substituted alkynylene. In certain embodiments, the linker comprises optionally substituted heteroalkylene. In certain embodiments, the linker comprises optionally substituted heteroalkenylene. In certain embodiments, the linker comprises optionally substituted heteroalkynylene. In certain embodiments, the linker comprises optionally substituted carbocyclylene. In certain embodiments, the linker comprises optionally substituted heterocyclylene. In certain embodiments, the linker comprises optionally substituted arylene. In certain embodiments, the linker comprises optionally substituted heteroarylene. In certain embodiments, the linker comprises optionally substituted acylene.

In certain embodiments, the linker is a cleavable linker. “Cleavable linker” as used herein refers to a linker capable of cleaving under physiological conditions. In certain embodiments, the linker is pH cleavable or cleavable by a protease, esterase, or intracellular disulfide reduction. In certain embodiments, the linker is cleavable by a protease. See, e.g., Bargh et al., Chem. Soc. Rev. 2019, 48(16), 4361-4374; Zheng Su et al., “Antibody-drug conjugates: Recent advances in linker chemistry”, Acta Pharmaceutica Sinica B, 2021; and Leriche et al., Bioorganic & Medicinal Chemistry, 2012, vol. 20, 571-582, the entire contents of each of which is incorporated herein by reference.

In certain embodiments, the linker is a peptidic linker. In certain embodiments, the linker is a cleavable peptidic linker. “Peptidic linker” as used herein refers to a linker comprising two or more amino acids linked via peptide bonds. In certain embodiments, the peptidic linker comprises —Y^{A}Y^{B}Y^{C}Y^{D}—(SEQ ID NO: 159), wherein:
Y^{A} is glycine, glutamic acid, or is absent;
Y^{B} is valine, phenylalanine, alanine, tyrosine, or glycine;
Y^{C} is citrulline, arginine, lysine, alanine, or glycine; and
Y^{D} is glycine or is absent.

In certain embodiments, the peptidic linker comprises -GGFG- (SEQ ID NO: 160). In certain embodiments, the peptidic linker comprises -GGG-. In certain embodiments, the peptidic linker comprises -EVC-. See, e.g., Anami et al., Nature Communications, 2018, 9, 2512, the entire contents of which is incorporated herein by reference.

In certain embodiments, the peptidic linker comprises -valine-citrulline- (i.e., -V-C-). Table 5 below shows non-limiting examples of such linkers, represented by linking reagent and the corresponding resulting linker structure.

TABLE 5
Examples of Linkers and Linking Reagents

| Linker | | Exemplary |
|---|---|---|
| Number | Linker Structure* | Linking Reagent |
| L1 | 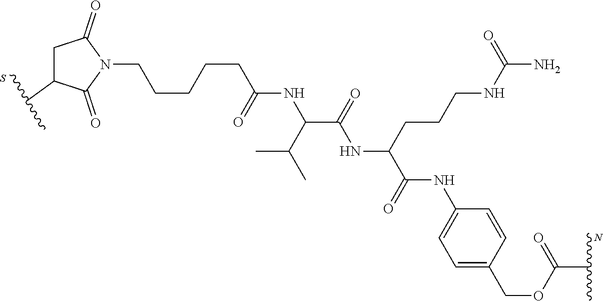 | Maleimidocaproyl-L-valine-L-citrulline-p-aminobenzyl alcohol p-nitrophenyl carbonate (Mc-Val-Cit-PABC-PNP) |
| | (maleimide-caproic acid-valine-citrulline-para- | |
| | aminobenzyl) | |
| L2 | 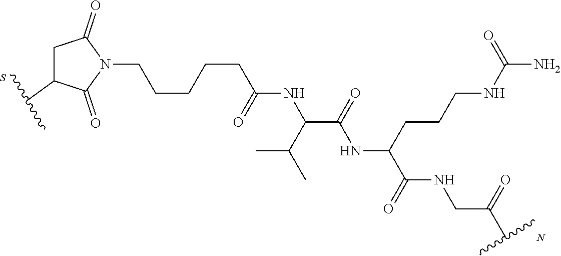 | Maleimidocaproyl-L-valine-L-citrulline-glycine (Mc-Val-Cit-Gly) |
| | (maleimide-caproic acid-valine-citrulline-glycine) | |
| L3 | 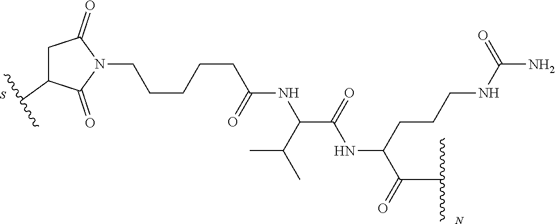 | Maleimidocaproyl-L-valine-L-citrulline (Mc-Val-Cit) |
| | (maleimide-caproic acid-valine-citrulline) | |
| L4 | 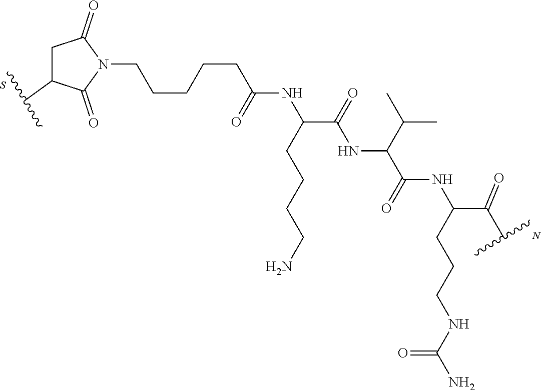 | Maleimidocaproyl-L-lysine-L-valine-L-citrulline (Mc- Lys-Val-Cit) |
| | (maleimide-caproic acid-lysine-valine-citrulline) | |
| L5 | 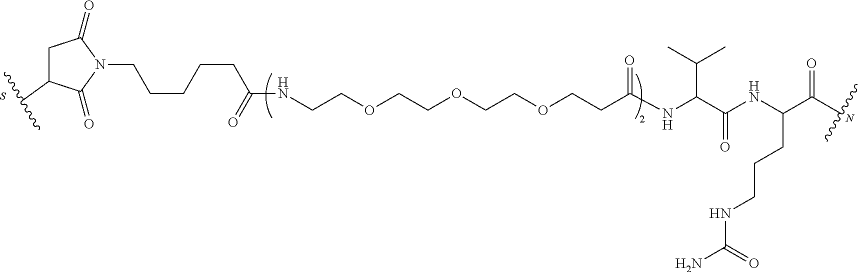 | Maleimidocaproyl-(PEG3)-(PEG3)-L-valine-L-citrulline (Mc-(PEG3)-(PEG3)- Val-Cit) |
| | (maleimide-caproic acid-(PEG3)-(PEG3)-valine- | |
| | citrulline) | |
| L6 | 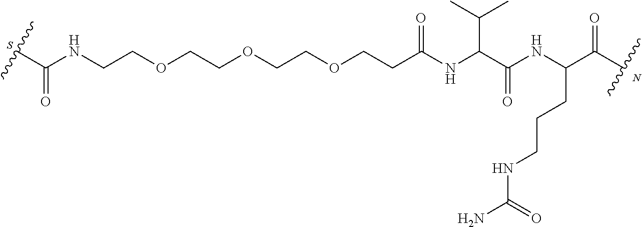 | Iodoacetamide-(PEG3)-L-valine-L-citrulline (IAA- (PEG3)-Val-Cit) |
| | (iodoacetamide-(PEG3)-valine-citrulline) | |
| L7 | 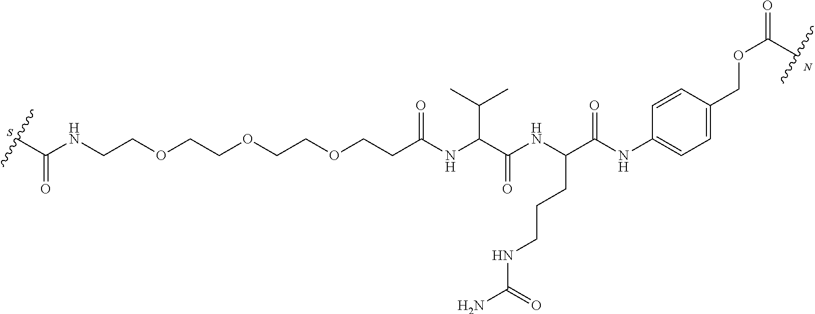 | Iodoacetamide-(PEG3)-L-valine-L-citrulline-p- aminobenzyl alcohol p- nitrophenyl carbonate (IAA- (PEG3)- Val-Cit-PABC-PNP) |
| | (iodoacetamide-(PEG3)-valine-citrulline-para- | |
| | aminobenzyl) | |
| L8 | 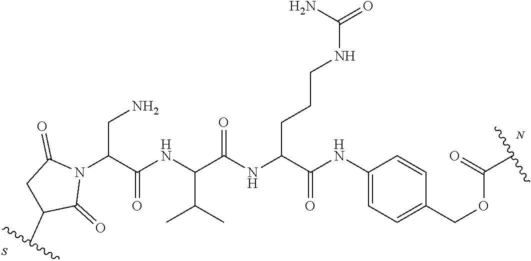 | Maleimido-L-2,3-diaminopropionate-L-valine-L- citrulline-p-aminobenzyl alcohol p-nitrophenyl carbonate |
| | (maleimide-2,3-diaminopropionate-valine-citrulline- | |
| | para-aminobenzyl) | |

*In the structures, S denotes the point of attachment to the antibody or antigen-binding fragment thereof;
N denotes the point of attachment to the N-terminus of the stapled peptide

In certain embodiments, the linker comprises a triazolylene moiety. As described herein, linkers comprising triazolylene moieties may be formed by azide-alkyne cycloaddition reactions. Non-limiting examples of triazole-containing linkers are shown below in Table 9.

TABLE 9
Examples of Trazolylene-Containing Linkers

| Linker | |
|---|---|
| Number | Linker Structure* |
| A1 | 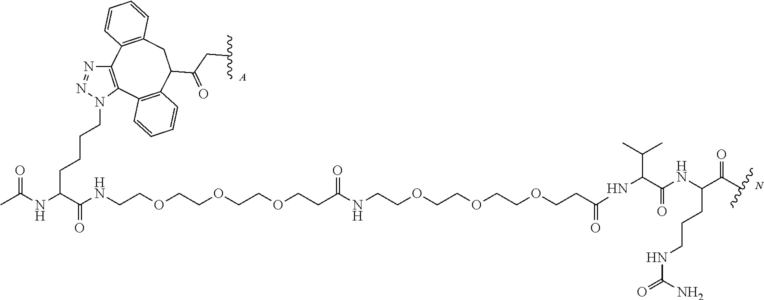 |
| A2 | 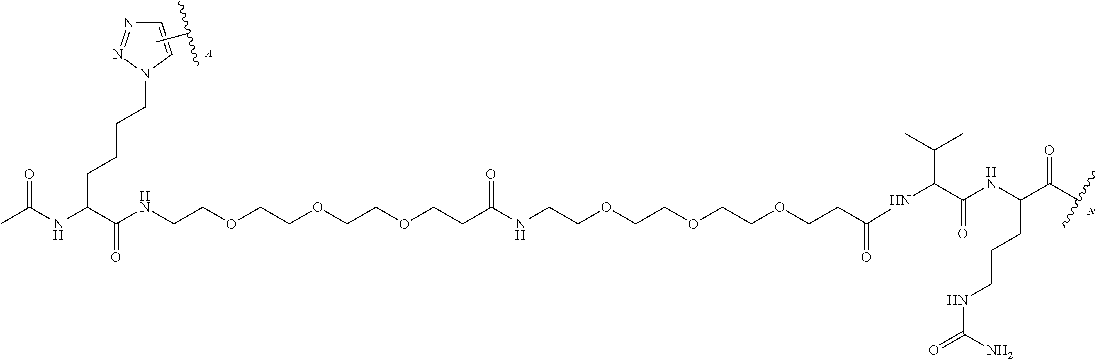 |
| A3 | 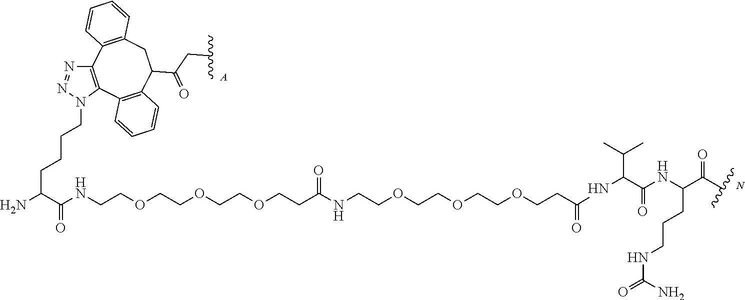 |
| A4 | 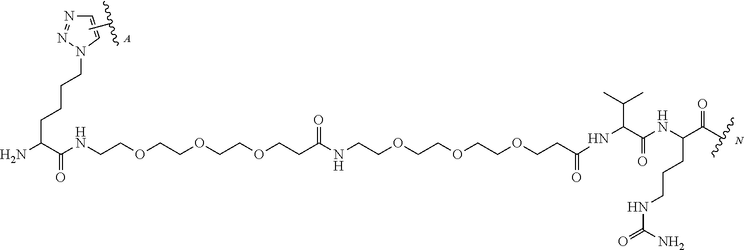 |
| A5 | 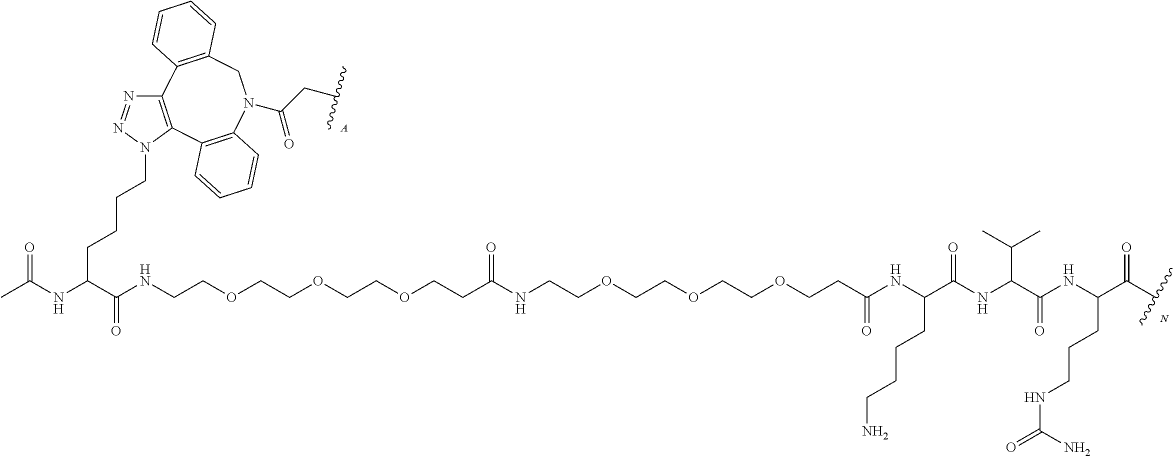 |
| A6 | 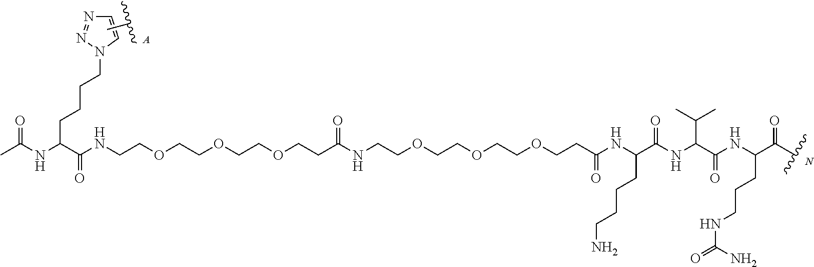 |
| A7 | 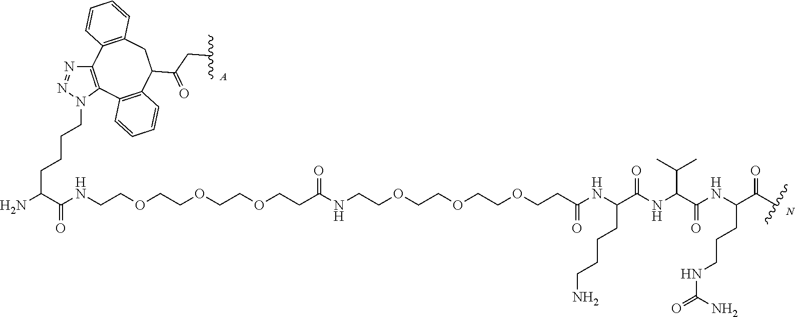 |
| A8 | 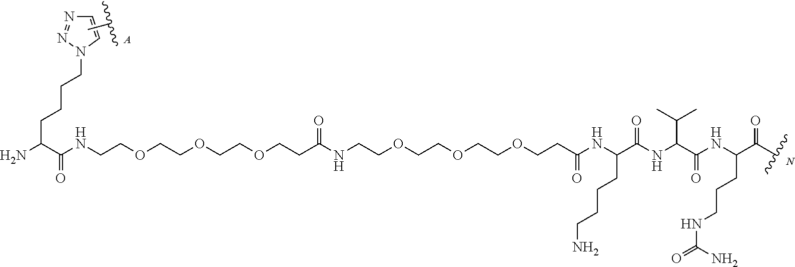 |
| A9 | 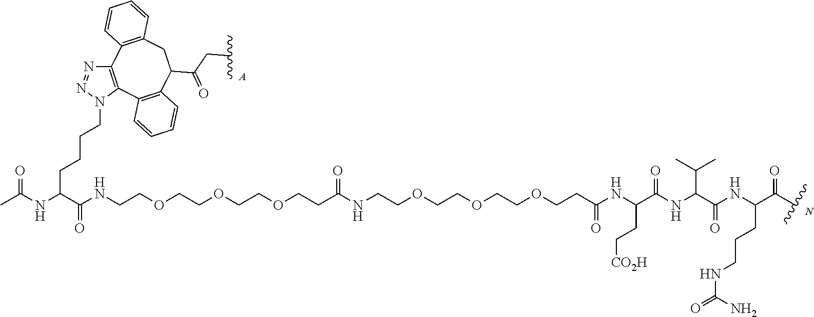 |
| A10 | 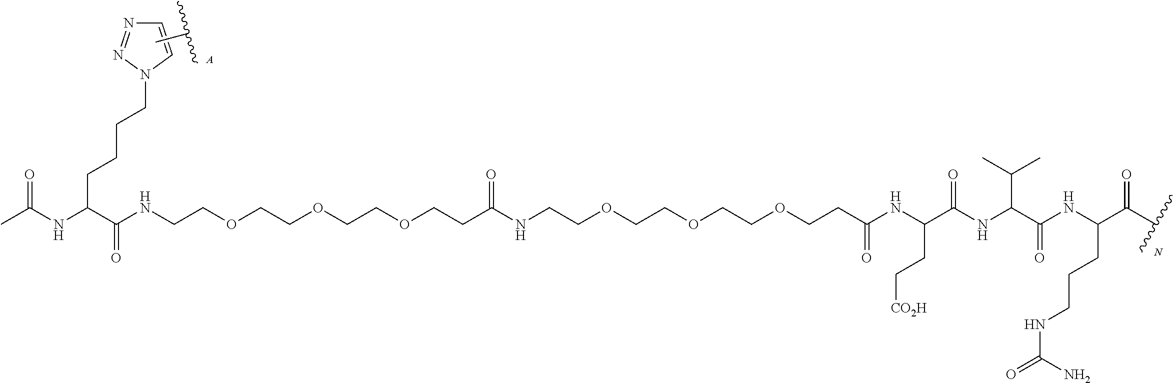 |
| A11 | 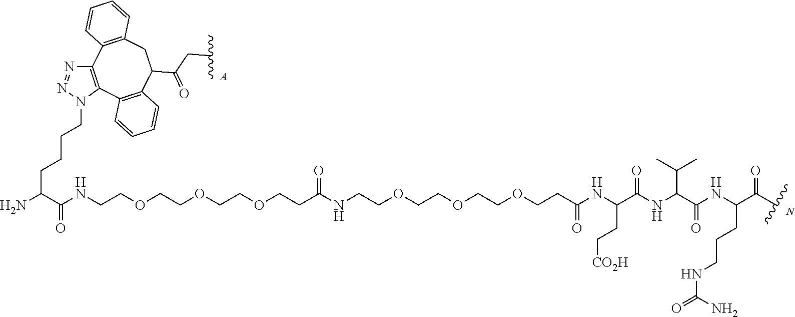 |
| A12 | 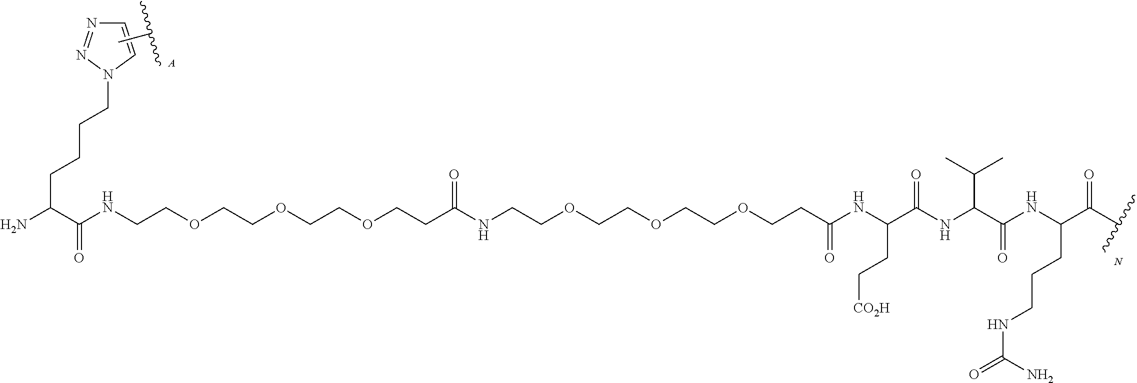 |
| A13 | 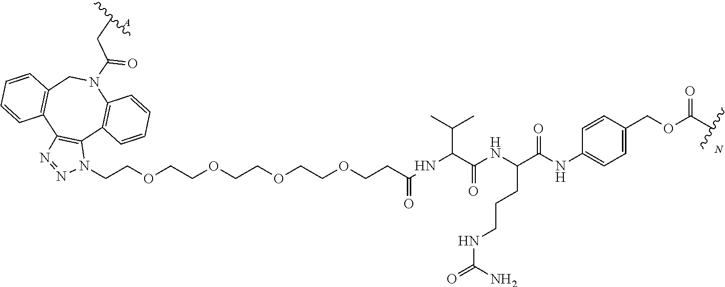 |
| A14 | 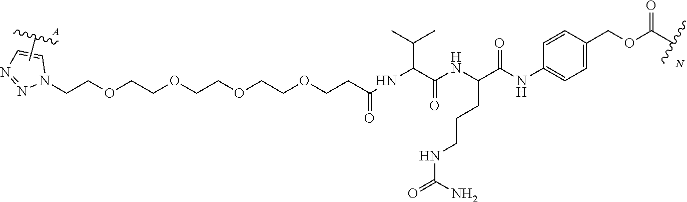 |
| A15 | 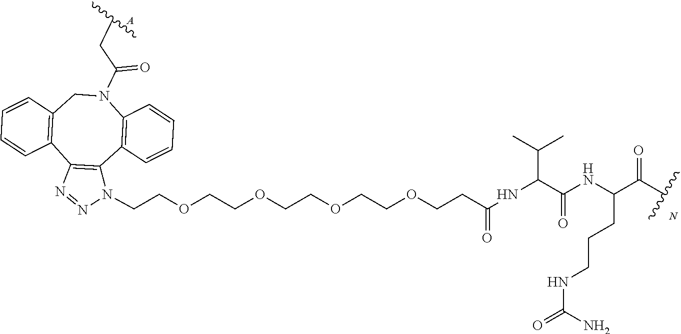 |
| A16 | 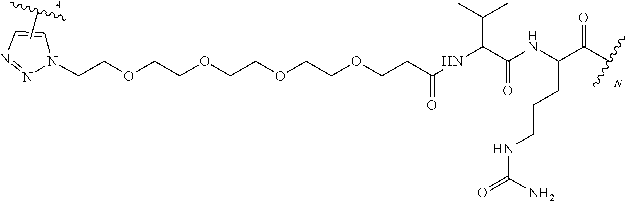 |
| A17 | 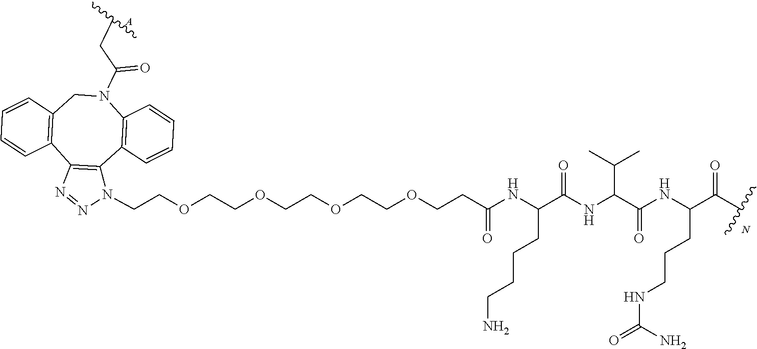 |
| A18 | 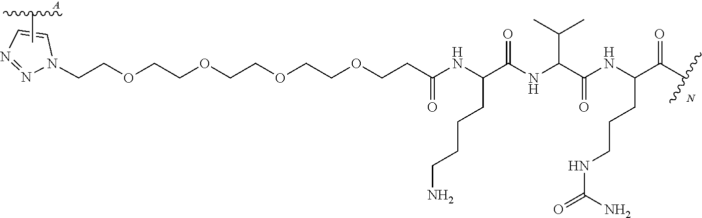 |
| A19 | 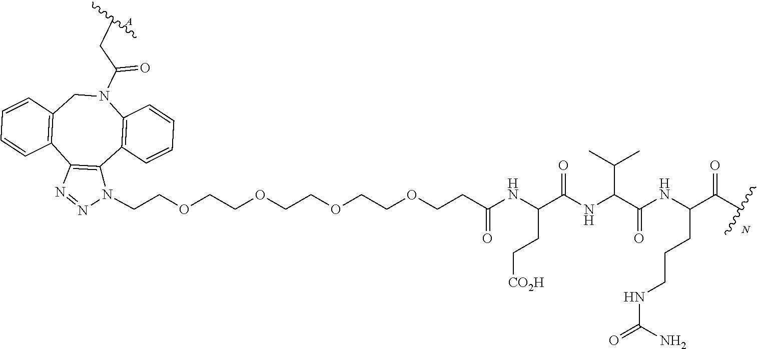 |
| A20 | 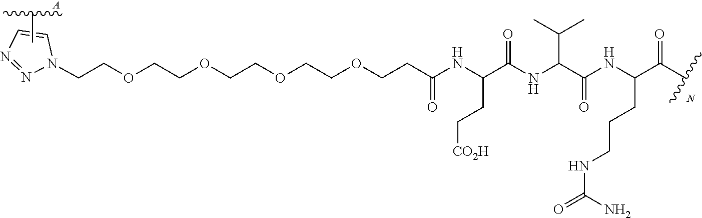 |

*In the structures, A denotes a point of linkage to the antibody or antigen-binding fragment thereof;
N denotes the point of attachment to the N-terminus of the stapled peptide

In certain embodiments, a SPAC provided herein includes 1 stapled peptide conjugated to the antibody or antigen-binding fragment thereof (i.e., a 1:1 stapled peptide to antibody ratio). In certain embodiments, 2 or more stapled peptides are conjugated to the antibody or antigen-binding fragment thereof (i.e., a 2:1 stapled peptide to antibody ratio or greater). In certain embodiments, 1 to 20 stapled peptides, inclusive, are conjugated to the antibody or antigen-binding fragment thereof (i.e., a 1:1 to 20:1 stapled peptide to antibody ratio, inclusive). In certain embodiments, 2 to 20 stapled peptides, inclusive, are conjugated to the antibody or antigen-binding fragment thereof (i.e., a 2:1 to 20:1 stapled peptide to antibody ratio, inclusive). In certain embodiments, 1 to 10 stapled peptides, inclusive, are conjugated to the antibody or antigen-binding fragment thereof (i.e., a 1:1 to 10:1 stapled peptide to antibody ratio, inclusive). In certain embodiments, 2 to 10 stapled peptides, inclusive, are conjugated to the antibody or antigen-binding fragment thereof (i.e., a 2:1 to 10:1 stapled peptide to antibody ratio, inclusive). In certain embodiments, 5 to 10 stapled peptides, inclusive, are conjugated to the antibody or antigen-binding fragment thereof (i.e., a 1:1 to 10:1 stapled peptide to antibody ratio, inclusive). In certain embodiments, the antibody or antigen-binding fragment thereof is conjugated to 1, 2, 3, 4, 5, 6, 7, 8, 9, 10, 11, 12, 13, 14, 15, 16, 17, 18, 19, or 20 stapled peptides. In certain embodiments, about 8 stapled are conjugated to the antibody or antigen-binding fragment thereof (i.e., an 8:1 stapled peptide to antibody ratio). In certain embodiments, the stapled peptides are the same or different, in any combination.

### ADDITIONAL EMBODIMENTS

Additional embodiments and examples of SPACs are provided below, including in Table 6.

In certain embodiments, the stapled peptide is a stapled anti-cancer peptide; and the antibody is a mAb antibody directed against HER2, or an antigen-binding fragment thereof. In certain embodiments, the stapled peptide is a stapled anti-cancer peptide; and the antibody is a mAb antibody directed against CD38, or an antigen-binding fragment thereof. In certain embodiments, the stapled peptide is a stapled anti-cancer peptide; and the antibody is trastuzumab. In certain embodiments, the stapled peptide is a stapled anti-cancer peptide; and the antibody is trastuzumab emtansine. In certain embodiments, the stapled peptide is a stapled anti-cancer peptide; and the antibody is daratumumab.

In certain embodiments, the stapled peptide is of SEQ ID NO: 48, or a pharmaceutically acceptable salt thereof; and the antibody is a mAb antibody directed against HER2, or an antigen-binding fragment thereof. In certain embodiments, the stapled peptide is of SEQ ID NO: 26, or a pharmaceutically acceptable salt thereof; and the antibody is a mAb antibody directed against HER2, or an antigen-binding fragment thereof. In certain embodiments, the stapled peptide is of SEQ ID NO: 5, or a pharmaceutically acceptable salt thereof, and the antibody is a mAb antibody directed against HER2, or an antigen-binding fragment thereof.

In certain embodiments, the stapled peptide is of SEQ ID NO: 48, or a pharmaceutically acceptable salt thereof; and the antibody is a mAb antibody directed against CD38, or an antigen-binding fragment thereof. In certain embodiments, the stapled peptide is of SEQ ID NO: 26, or a pharmaceutically acceptable salt thereof; and the antibody is a mAb antibody directed against CD38, or an antigen-binding fragment thereof. In certain embodiments, the stapled peptide is of SEQ ID NO: 5, or a pharmaceutically acceptable salt thereof; and the antibody is a mAb antibody directed against CD38, or an antigen-binding fragment thereof.

In certain embodiments, the stapled peptide is of SEQ ID NO: 26, or a pharmaceutically acceptable salt thereof; and the antibody is trastuzumab (e.g., SPAC 1 in Table 6 below). In certain embodiments, the stapled peptide is of SEQ ID NO: 26, or a pharmaceutically acceptable salt thereof; and the antibody is trastuzumab emtansine (e.g., SPAC 17 and SPAC 18 in Table 6 below). In certain embodiments, the stapled peptide is of SEQ ID NO: 48, or a pharmaceutically acceptable salt thereof; and the antibody is trastuzumab (e.g., SPAC 2 in Table 6 below). In certain embodiments, the stapled peptide is of SEQ ID NO: 26, or a pharmaceutically acceptable salt thereof; and the antibody is daratumumab (e.g., SPAC 3 in Table 6 below). In certain embodiments, the stapled peptide is of SEQ ID NO: 48, or a pharmaceutically acceptable salt thereof; and the antibody is daratumumab (e.g., SPAC 4 in Table 6 below). In certain embodiments, the stapled peptide is of SEQ ID NO: 5, or a pharmaceutically acceptable salt thereof; and the antibody is trastuzumab (e.g., SPAC 5 in Table 6 below). In certain embodiments, the stapled peptide is of SEQ ID NO: 5, or a pharmaceutically acceptable salt thereof; and the antibody is daratumumab (e.g., SPAC 6 in Table 6 below).

TABLE 6
Examples of SPACs

| | Stapled | | | | | |
|---|---|---|---|---|---|---|
| | Peptide | | | Stapled | | |
| SPAC | SEQ ID | X¹-X² | X³-X⁴ | Peptide C- | Linker | |
| # | NO. | crosslink | Crosslink | Terminus | Number | Antibody |
| 1 | 26 | alk | alk | —NH₂ | L1 | trastuzumab |
| 2 | 48 | alk | alk | —NH₂ | L1 | trastuzumab |
| 3 | 26 | alk | alk | —NH₂ | L1 | daratumumab |
| 4 | 48 | alk | alk | —NH₂ | L1 | daratumumab |
| 5 | 5 | alk | alk | —NH₂ | L1 | trastuzumab |
| 6 | 5 | alk | alk | —NH₂ | L1 | daratumumab |
| 17 | 26 | alk | alk | —NH₂ | L1 | trastuzumab |
| | | | | | | emtansine |
| 18 | 26 | alk | alk | —NH₂ | L5 | trastuzumab |
| | | | | | | emtansine |

Additional embodiments and examples of SPACs are provided below, including in Table 7.

In certain embodiments, the stapled peptide is an MDM2 inhibitor, or a pharmaceutically acceptable salt thereof; and the antibody is a mAb antibody directed against HER2, or an antigen-binding fragment thereof. In certain embodiments, the stapled peptide is ATSP-7041, or a pharmaceutically acceptable salt thereof; and the antibody is a mAb antibody directed against HER2, or an antigen-binding fragment thereof. In certain embodiments, the stapled peptide is ATSP-7041, or a pharmaceutically acceptable salt thereof; and the antibody is trastuzumab. In certain embodiments, the stapled peptide is ATSP-7041, or a pharmaceutically acceptable salt thereof; and the antibody is trastuzumab emtansine.

In certain embodiments, the stapled peptide comprises any one of SEQ ID NOs: 161-166, or a pharmaceutically acceptable salt thereof; and the antibody is a mAb antibody directed against HER2, or an antigen-binding fragment thereof. In certain embodiments, the stapled peptide comprises any one of SEQ ID NOs: 161-166, or a pharmaceutically acceptable salt thereof, and the antibody is trastuzumab. In certain embodiments, the stapled peptide comprises any one of SEQ ID NOs: 161-166, or a pharmaceutically acceptable salt thereof; and the antibody is trastuzumab emtansine. In certain embodiments, the stapled peptide is ATSP-7041 Cba10L, or a pharmaceutically acceptable salt thereof; and the antibody is a mAb antibody directed against HER2, or an antigen-binding fragment thereof. In certain embodiments, the stapled peptide is ATSP-7041 Cba10L, or a pharmaceutically acceptable salt thereof, and the antibody is trastuzumab (e.g., SPAC 7 in Table 7 below). In certain embodiments, the stapled peptide is ATSP-7041 Cba10L, or a pharmaceutically acceptable salt thereof, and the antibody is trastuzumab emtansine (e.g., SPAC 9 in Table 7 below).

In certain embodiments, the stapled peptide is an MDM2 inhibitor, or a pharmaceutically acceptable salt thereof; and the antibody is a mAb antibody directed against CD38, or an antigen-binding fragment thereof. In certain embodiments, the stapled peptide is ATSP-7041, or a pharmaceutically acceptable salt thereof; and the antibody is a mAb antibody directed against CD38, or an antigen-binding fragment thereof. In certain embodiments, the stapled peptide ATSP-7041, or a pharmaceutically acceptable salt thereof; and the antibody is daratumumab.

In certain embodiments, the stapled peptide comprises any one of SEQ ID NOs: 161-166, or a pharmaceutically acceptable salt thereof; and the antibody is a mAb antibody directed against CD38, or an antigen-binding fragment thereof. In certain embodiments, the stapled peptide comprises any one of SEQ ID NOs: 161-166, or a pharmaceutically acceptable salt thereof, and the antibody is daratumumab. In certain embodiments, the stapled peptide is ATSP-7041 Cba10L, or a pharmaceutically acceptable salt thereof; and the antibody is a mAb antibody directed against CD38, or an antigen-binding fragment thereof. In certain embodiments, the stapled peptide ATSP-7041 Cba10L, or a pharmaceutically acceptable salt thereof, and the antibody is daratumumab (e.g., SPAC 8 in Table 7 below).

In certain embodiments, the stapled peptide is an MCL-1 inhibitor; and the antibody is a mAb antibody directed against HER2, or an antigen-binding fragment thereof. In certain embodiments, the stapled peptide is MCL-1 SAHB_{D}, or a pharmaceutically acceptable salt thereof, and the antibody is a mAb antibody directed against HER2, or an antigen-binding fragment thereof. In certain embodiments, the stapled peptide is MCL-1 SAHB_{D}, or a pharmaceutically acceptable salt thereof; and the antibody is trastuzumab (e.g., SPAC 10 in Table 7 below). In certain embodiments, the stapled peptide is MCL-1 SAHB_{D}, or a pharmaceutically acceptable salt thereof; and the antibody is trastuzumab emtansine (e.g., SPAC 12 in Table 7 below).

In certain embodiments, the stapled peptide is an MCL-1 inhibitor; and the antibody is a mAb antibody directed against CD38, or an antigen-binding fragment thereof. In certain embodiments, the stapled peptide is MCL-1 SAHB_{D}, or a pharmaceutically acceptable salt thereof, and the antibody is a mAb antibody directed against CD38, or an antigen-binding fragment thereof. In certain embodiments, the stapled peptide is MCL-1 SAHB_{D}, or a pharmaceutically acceptable salt thereof; and the antibody is daratumumab (e.g., SPAC 11 in Table 7 below).

In certain embodiments, the stapled peptide is a β-catenin inhibitor; and the antibody is a mAb antibody directed against HER2, or an antigen-binding fragment thereof. In certain embodiments, the stapled peptide is SAH-BCL9_{B}, or a pharmaceutically acceptable salt thereof, and the antibody is a mAb antibody directed against HER2, or an antigen-binding fragment thereof. In certain embodiments, the stapled peptide is SAH-BCL9_{B}, or a pharmaceutically acceptable salt thereof; and the antibody is trastuzumab (e.g., SPAC 13 in Table 7 below).

In certain embodiments, the stapled peptide is a β-catenin inhibitor; and the antibody is a mAb antibody directed against CD38, or an antigen-binding fragment thereof. In certain embodiments, the stapled peptide is SAH-BCL9_{B}, or a pharmaceutically acceptable salt thereof, and the antibody is a mAb antibody directed against CD38, or an antigen-binding fragment thereof. In certain embodiments, the stapled peptide is SAH-BCL9_{B}, or a pharmaceutically acceptable salt thereof; and the antibody is daratumumab (e.g., SPAC 14 in Table 7 below).

In certain embodiments, the stapled peptide is xStAx-34, or a pharmaceutically acceptable salt thereof, and the antibody is a mAb antibody directed against HER2, or an antigen-binding fragment thereof. In certain embodiments, the stapled peptide is xStAx-34, or a pharmaceutically acceptable salt thereof; and the antibody is trastuzumab (e.g., SPAC 15 in Table 7 below).

In certain embodiments, the stapled peptide is xStAx-34, or a pharmaceutically acceptable salt thereof, and the antibody is a mAb antibody directed against CD38, or an antigen-binding fragment thereof. In certain embodiments, the stapled peptide is xStAx-34, or a pharmaceutically acceptable salt thereof; and the antibody is daratumumab (e.g., SPAC 16 in Table 7 below).

TABLE 7
Additional Examples of SPACs

| SPAC # | Stapled Peptide | Linker Number | Antibody |
|---|---|---|---|
| 7 | ATSP-7041 Cba10L | L3 | trastuzumab |
| 8 | ATSP-7041 Cba10L | L3 | daratumumab |
| 9 | ATSP-7041 Cba10L | L3 | trastuzumab emtansine |
| 10 | MCL-1 SAHB_{D} | L2 | trastuzumab |
| 11 | MCL-1 SAHB_{D} | L2 | daratumumab |
| 12 | MCL-1 SAHB_{D} | L2 | trastuzumab emtansine |
| 13 | SAH-BCL9_{B} | L2 | trastuzumab |
| 14 | SAH-BCL9_{B} | L2 | daratumumab |
| 15 | xStAx-34 | L2 | trastuzumab |
| 16 | xStAx-34 | L2 | daratumumab |

In certain embodiments, the stapled peptide is an MDM2 inhibitor; and the antibody is an antibody directed against TM4SF1, or an antigen-binding fragment thereof. In certain embodiments, the stapled peptide comprises any one of SEQ ID NOs: 161-166, or a pharmaceutically acceptable salt thereof; and the antibody is an antibody directed against TM4SF1, or an antigen-binding fragment thereof. In certain embodiments, the stapled peptide is of any one of SEQ ID NOs: 164-166, or a pharmaceutically acceptable salt thereof; and the antibody is an antibody directed against TM4SF1, or an antigen-binding fragment thereof. In certain embodiments, the stapled peptide is of any one of SEQ ID NOs: 164-166, or a pharmaceutically acceptable salt thereof; the antibody is an antibody directed against TM4SF1, or an antigen-binding fragment thereof, and linker comprises any one of A1-A20.

### Methods of Preparing Stapled Peptide-Antibody Conjugates (SPACs)

Also provided herein are methods of preparing the stapled peptide-antibody conjugates (SPACs) described herein. In certain embodiments, a stapled peptide is conjugated to an antibody or antigen-binding fragment thereof using a linking reagent. “Linking reagent” as used herein refers to a molecule comprising two reactive moieties, one capable of reacting with a reactive moiety on the antibody to form at least one covalent bond, and another capable of reacting with a reactive moiety on the stapled peptide to form at least one covalent bond.

For example, a linking reagent may comprise (i) a moiety capable of reacting with a thiol (e.g., cysteine residue) or amine (e.g., lysine residue) of the antibody; and (ii) a moiety capable with reacting with the N-terminal amine of the stapled peptide. For example, a linking reagent may comprise (i) a maleimide or iodoacetamide (e.g., capable of reacting with a thiol (e.g., cysteine residue) or amine (e.g., lysine residue) of the antibody); and (ii) a carboxylic acid or ester (e.g., capable with reacting with the N-terminal amine of the stapled peptide). Non-limiting examples of linking reagents are provided in Table 5.

In certain embodiments, a method for preparing a stapled peptide-antibody conjugate (SPAC) described herein comprises the steps of:
(a) contacting a stapled peptide with a linking reagent under conditions sufficient to conjugate the linking reagent with the stapled peptide, thereby forming a stapled peptide-linking reagent intermediate; and
(b) contacting the stapled peptide-linking reagent intermediate with an antibody or antigen-binding fragment thereof under conditions sufficient to conjugate the stapled peptide-linking reagent intermediate to an antibody or antigen-binding fragment thereof, thereby forming the stapled peptide-antibody conjugate (SPAC).

In certain embodiments, a method for preparing a stapled peptide-antibody conjugate (SPAC) described herein comprises the steps of:
(a) contacting an antibody or antigen binding fragment thereof with a linking reagent under conditions sufficient to conjugate the linking reagent with antibody or antigen-binding fragment thereof, thereby forming an antibody-linking reagent intermediate; and
(b) contacting the antibody-linking reagent intermediate with a stapled peptide under conditions sufficient to conjugate the antibody-linking reagent intermediate to a stapled peptide, thereby forming a stapled peptide-antibody conjugate (SPAC).

In other embodiments, a method for preparing a stapled peptide-antibody conjugate (SPAC) described herein comprises a step of contacting a stapled peptide comprising a first reactive moiety with an antibody or antigen-binding fragment thereof comprising a second reactive moiety under conditions sufficient to form at least one covalent bond between the first reactive moiety and the second reactive moiety, thereby forming the SPAC. In certain embodiments, the first reactive moiety and second reactive moiety are “click chemistry” handles capable of reacting with each other to form one or more covalent bonds therebetween.

“Click chemistry” is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395. Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2]cycloaddition). Examples of alkyne-azide reactions can be found in, e.g., Kolb, Finn and Sharpless Angewandte Chemie International Edition (2001) 40: 2004-2021; Kolb and Sharpless, Drug Discov Today (2003) 24: 1128-1137; and Evans, Australian Journal of Chemistry (2007) 60: 384-395.

In certain embodiments, the first reactive moiety is an azide; and the second reactive moiety is an alkyne. In certain embodiments, the first reactive moiety is an alkyne and the second reactive moiety is an azide.

For example, a method for preparing a stapled peptide-antibody conjugate (SPAC) described herein comprises a step of contacting a stapled peptide comprising an azide with an antibody or antigen-binding fragment thereof comprising an alkyne under conditions sufficient to form a triazolylene-containing linker, thereby forming the SPAC.

In certain embodiments, the stapled peptide comprising an azide comprises one of the following formulae (e.g., to form any one of linkers A1-A20):

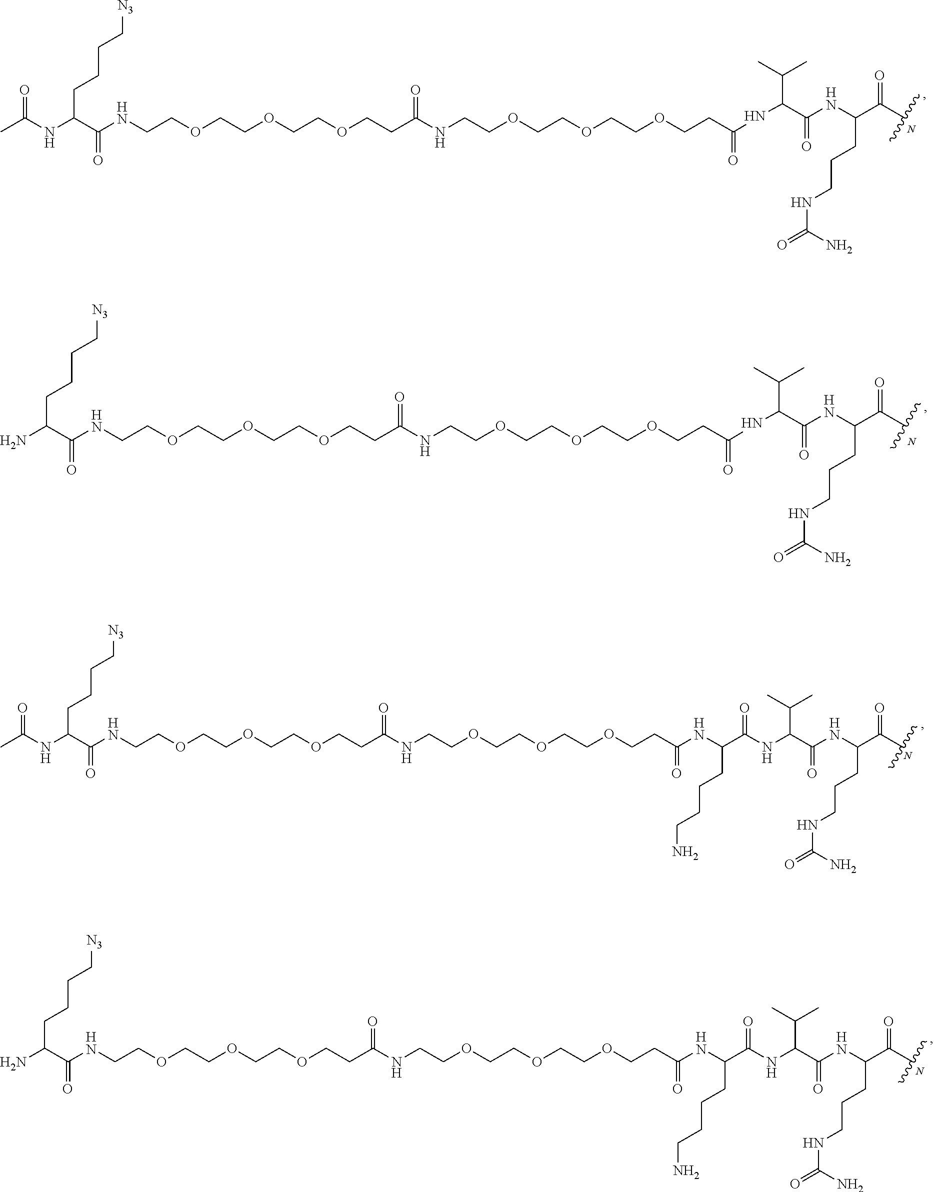

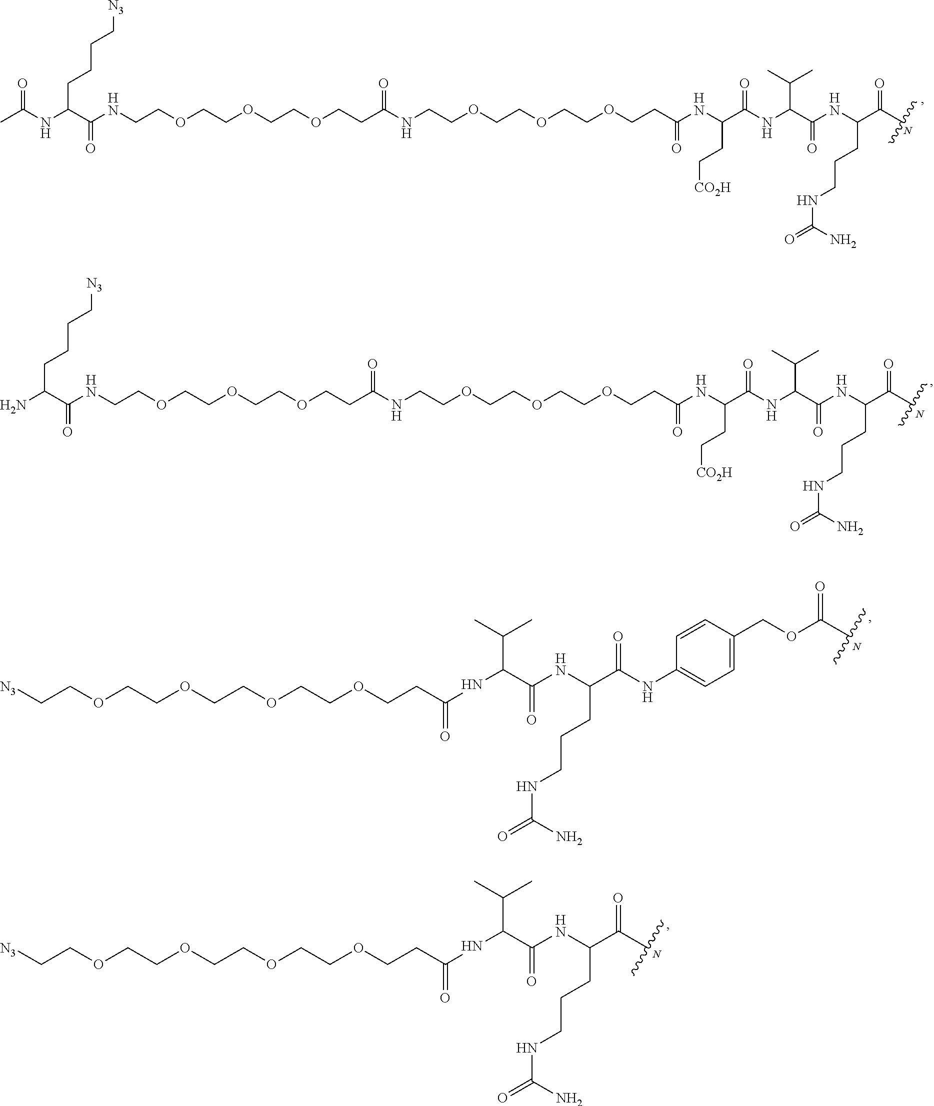

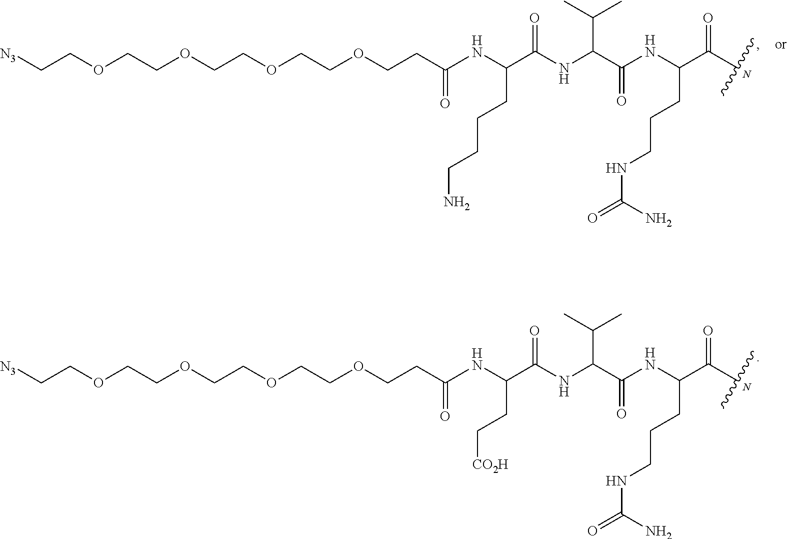

Provided herein are stapled peptides comprising any one of SEQ ID NOs: 161-166, or a pharmaceutically acceptable salt thereof; and further comprising an azide. For example, provided herein are stapled peptides comprising any one of SEQ ID NOs: 161-166, or a pharmaceutically acceptable salt thereof; and further comprising one of the following formulae at the N-terminus of the amino acid sequence:

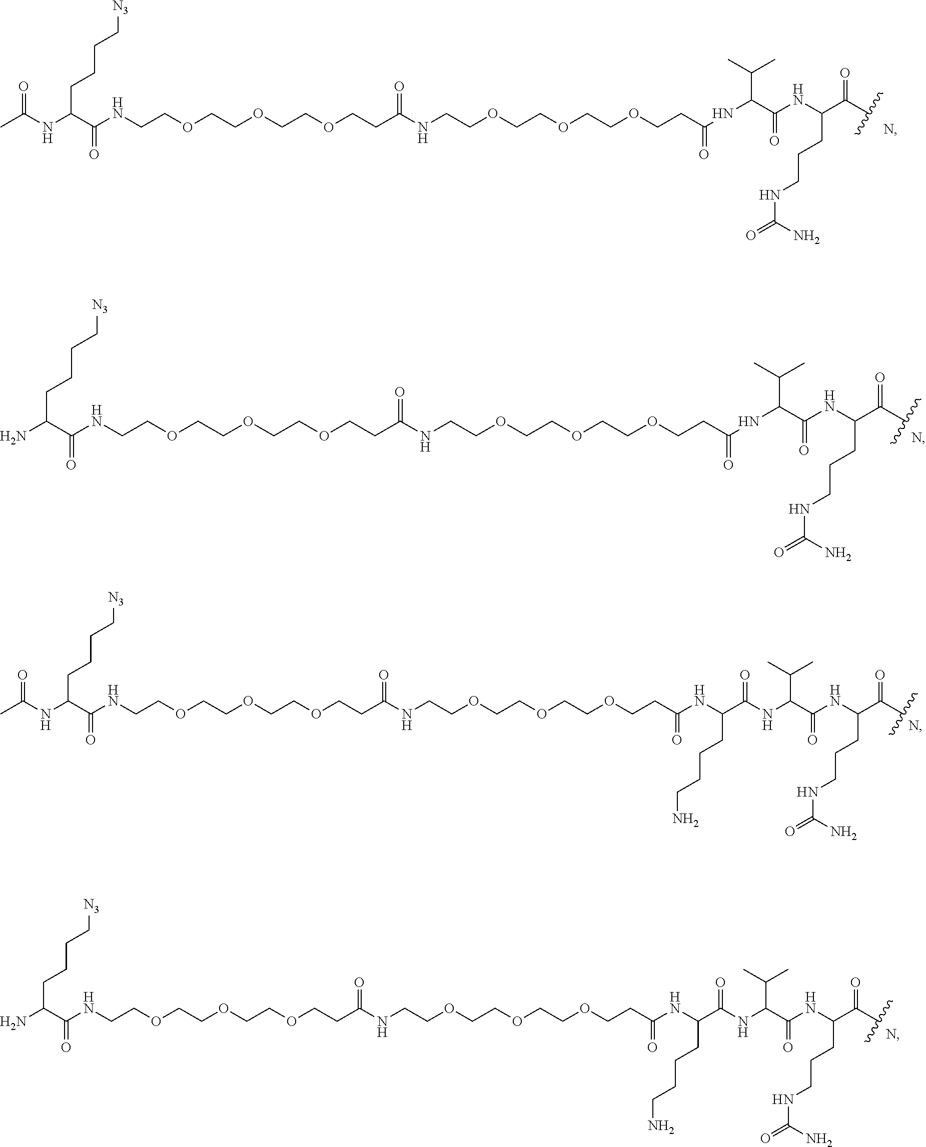

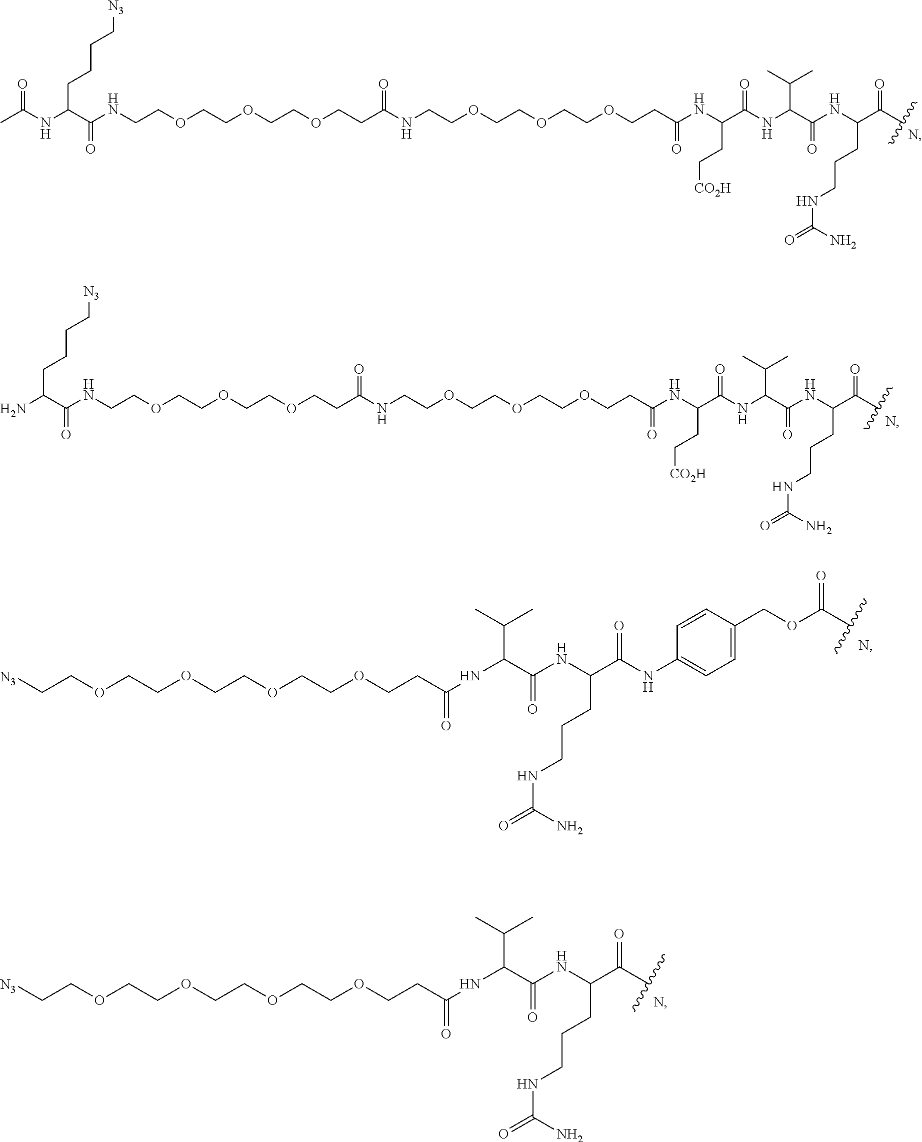

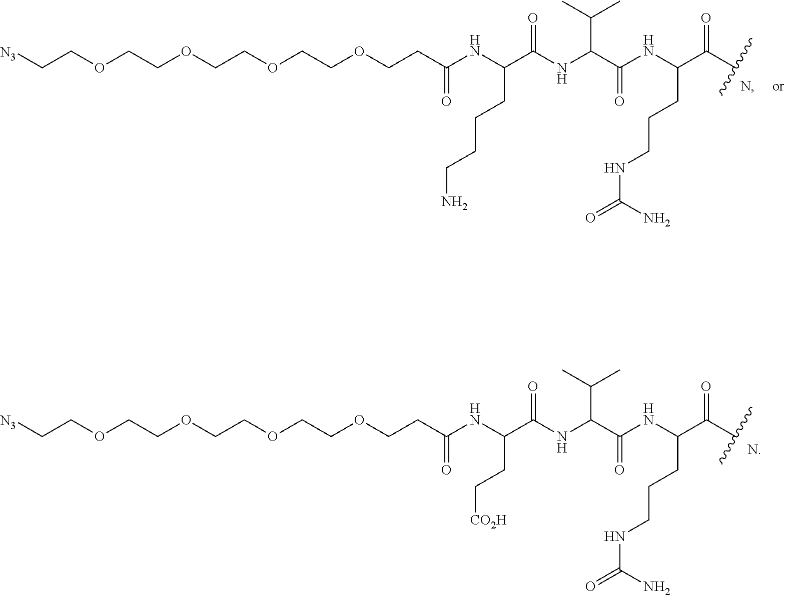

In certain embodiments, the antibody or antigen binding fragment thereof comprises a terminal alkyne (e.g., for use in copper-promoted cycloaddition with an azide). In certain embodiments, the antibody or antigen binding fragment thereof comprises cyclic alkyne (e.g., for use in strain-promoted (e.g., copper-free) cycloaddition with an azide). Non-limiting examples of cyclic alkyne moieties include DBCO and sulfo-DBCO:

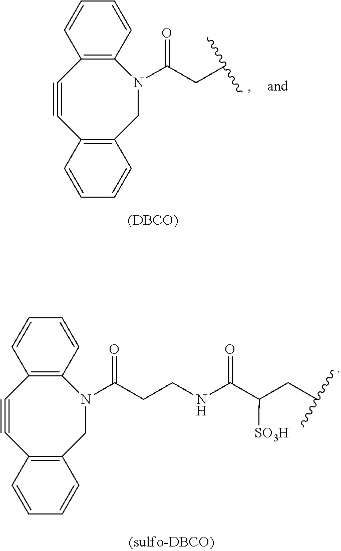

### Pharmaceutical Compositions, Kits, and Administration

The present disclosure provides pharmaceutical compositions comprising a SPAC disclosed herein. The pharmaceutical composition may comprise one or more pharmaceutically acceptable carriers/excipients. In certain embodiments, a SPAC described herein is provided in an effective amount in the pharmaceutical composition. In certain embodiments, the effective amount is a therapeutically effective amount (e.g., for treating cancer in a subject and/or inhibiting tumor growth in a subject). In certain embodiments, the effective amount is a prophylactically effective amount.

Pharmaceutical compositions described herein can be prepared by any method known in the art of pharmacology. In general, such preparatory methods include bringing the SPAC described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient. The composition may comprise between 0.1% and 50% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

In certain embodiments, the formulation comprises a polymer excipient. In certain embodiments, the formulation comprises a polyether. In certain embodiments, the formulation comprises polyethylene glycol (PEG) (e.g., PEG200, PEG300, PEG400, and the like).

Liquid dosage forms for parenteral administration include pharmaceutically acceptable emulsions, microemulsions, solutions, and suspensions. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables. In certain embodiments, the carrier is a buffered aqueous solution.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

SPACs provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The SPACs and compositions provided herein can be administered by any route, including, parenteral, enteral (e.g., oral), intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate a certain route of administration).

The exact amount of a SPAC required to achieve an effective amount will vary from subject to subject, depending, for example, on species, age, and general condition of a subject, severity of the side effects or disorder, identity of the particular SPAC, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a SPAC described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A SPAC or composition, as described herein, can be administered in combination with one or more additional pharmaceutical agents (e.g., therapeutically and/or prophylactically active agents). The SPACs or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a SPAC described herein and an additional pharmaceutical agent shows a synergistic effect that is absent in a pharmaceutical composition including one of the SPAC and the additional pharmaceutical agent, but not both. In some embodiments, the additional pharmaceutical agent achieves a desired effect for the same disorder. In some embodiments, the additional pharmaceutical agent achieves different effects.

The SPAC or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), SPACs, proteins, carbohydrates, monosaccharides, oligosaccharides, polysaccharides, nucleoproteins, mucoproteins, lipoproteins, synthetic SPACs or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

The additional pharmaceutical agents include, but are not limited to, anti-proliferative agents, anti-cancer agents, anti-angiogenesis agents, steroidal or non-steroidal anti-inflammatory agents (NSAIDs), immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

In certain embodiments, the additional pharmaceutical agent is an anti-cancer agent. “Anti-cancer agents” encompass biotherapeutic anti-cancer agents as well as chemotherapeutic agents.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunomodulatory agents (e.g., IL-1, 2, 4, 6, or 12), immune cell growth factors (e.g., GM-CSF) and antibodies (e.g. Herceptin (trastuzumab), T-DM1, AVASTIN (bevacizumab), ERBITUX (cetuximab), Vectibix (panitumumab), Rituxan (rituximab), Bexxar (tositumomab), as well as those listed in Table 2).

Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DI-FR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

Each additional pharmaceutical agent may be administered at a dose and/or on a time schedule determined for that pharmaceutical agent. The additional pharmaceutical agents may also be administered together with each other and/or with the SPAC or composition described herein in a single dose or composition or administered separately in different doses or compositions. The particular combination to employ in a regimen will take into account compatibility of the SPAC described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the SPAC is used in combination with one or more different treatment modalities such as radiation therapy or surgery.

Also encompassed by the disclosure are kits (e.g., pharmaceutical packs). The kits provided may comprise a pharmaceutical composition or SPAC described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or SPAC described herein. In some embodiments, the pharmaceutical composition or SPAC described herein provided in the first container and the second container are combined to form one unit dosage form. Thus, in one aspect, provided are kits including a first container comprising a SPAC or pharmaceutical composition described herein. In certain embodiments, the kits are useful for treating a disease (e.g., cancer) in a subject in need thereof. In certain embodiments, the kits are useful for preventing a disease in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits provide instructions for treating a disease (e.g., cancer) in a subject in need thereof. In certain embodiments, the kits provide instructions for preventing a disease (e.g., cancer) in a subject in need thereof. A kit described herein may include one or more additional pharmaceutical agents described herein as a separate composition.

### Methods of Treatment and Uses

Stapled-peptide antibody conjugates (SPACs) provided herein can deliver biologically active stapled peptides to cells, and are therefore useful in the treatment and/or prevention of diseases (e.g., proliferative diseases(e.g., cancer), infectious diseases).

Provided herein are methods of treating and/or preventing a disease in a subject comprising administering to the subject a therapeutically and/or prophylactically effective amount of a SPAC provided herein, or a pharmaceutical composition thereof. Also provided herein are SPACs, and pharmaceutical compositions thereof, for use in treating and/or preventing a disease in a subject. Also provided herein are uses of SPACs, and pharmaceutical compositions thereof, for the manufacture of medicaments. In certain embodiments, the disease is a proliferative disease (e.g., cancer), infectious disease (e.g., bacterial infection), inflammatory disease, or autoimmune disease.

In certain embodiments, the disease is a proliferative disease (e.g., cancer). Provided herein are methods of treating a proliferative disease (e.g., cancer) in a subject comprising administering to the subject a therapeutically effective amount of a SPAC provided herein, or a pharmaceutical composition thereof. Also provided herein are SPACs, and pharmaceutical compositions thereof, for use in treating a proliferative disease (e.g., cancer) in a subject. Also provided herein are uses of SPACs, and pharmaceutical compositions thereof, for the manufacture of medicaments for treating proliferative diseases (e.g., cancer). In certain embodiments, the proliferative disease is cancer.

A “proliferative disease” refers to a disease that occurs due to abnormal growth or extension by the multiplication of cells (See, e.g., Walker, Cambridge Dictionary of Biology; Cambridge University Press: Cambridge, UK, 1990). A proliferative disease may be associated with: (1) the pathological proliferation of normally quiescent cells; (2) the pathological migration of cells from their normal location (e.g., metastasis of neoplastic cells); (3) the pathological expression of proteolytic enzymes such as the matrix metalloproteinases (e.g., collagenases, gelatinases, and elastases); or (4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasia. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located.

The term “cancer” refers to a class of diseases characterized by the development of abnormal cells that proliferate uncontrollably and have the ability to infiltrate and destroy normal body tissues. In certain embodiments, the cancer is a solid tumor. In certain embodiments, the cancer is a hematopoietic cancer (i.e., hematological cancer).

In certain embodiments, the cancer is a hematopoietic cancer (e.g., leukemia (e.g., acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), chronic lymphocytic leukemia (CLL) (e.g., B-cell CLL, T-cell CLL)); lymphoma (e.g., Hodgkin lymphoma (HL) (e.g., B-cell HL, T-cell HL)), non-Hodgkin lymphoma (NHL) (e.g., B-cell NHL such as diffuse large cell lymphoma (DLCL) (e.g., diffuse large B-cell lymphoma)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome)), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); heavy chain disease (e.g., alpha chain disease, gamma chain disease, mu chain disease); a myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); multiple myeloma (MM); plasma cell neoplasia; familiar hypereosinophilia; inflammatory myofibroblastic tumors; immunocytic amyloidosis). In certain embodiments, the cancer is leukemia. In certain embodiments, the cancer is acute lymphoblastic leukemia (ALL). In certain embodiments, the cancer is early T-cell precursor (ETP)-acute lymphoblastic leukemia (ALL).

In certain embodiments, the cancer is musculoskeletal cancer (e.g., bone cancer (e.g., osteosarcoma, osteoid osteoma, malignant fibrous histiocytoma, Ewing's sarcoma, chordoma, malignant giant cell tumor chordoma, chondrosarcoma osteochondroma, benign chondroma, chondroblastoma chondromyxofibroma, myelodysplastic syndrome (MDS)), muscle cancer (e.g., rhabdomyosarcoma, rhabdomyoma), connective tissue cancer, synovioma).

In certain embodiments, the cancer is a nervous system cancer (e.g., brain cancer (e.g., astrocytoma, medulloblastoma, glioma (e.g., astrocytoma, oligodendroglioma), glioblastomas, glioblastoma multiform, medulloblastoma, ependymoma, germinoma (i.e., pinealoma), oligodendroglioma, schwannoma, retinoblastoma, congenital tumors, craniopharyngioma), spinal cord cancer, neurofibroma (e.g., neurofibromatosis (NF) type 1 or type 2, schwannomatosis), neuroblastoma, primitive neuroectodermal tumors (PNT), meningeal cancer (e.g., meningioma, meningiosarcoma, gliomatosis), skull cancer, acoustic neuroma, ependymoma, hemangioblastoma, ocular cancer (e.g., intraocular melanoma, retinoblastoma)). In certain embodiments, the disease to be treated is a brain tumor. In certain embodiments, the disease is pleomorphic xenoanthrocytoma (PXA). In certain embodiments, the disease is pediatric pleomorphic xenoanthrocytoma (PXA).

In certain embodiments, the cancer is selected from endocrine/exocrine cancers (e.g., thyroid cancer (e.g., papillary thyroid carcinoma, follicular thyroid carcinoma; medullary thyroid carcinoma, multiple endocrine neoplasia type 2A, multiple endocrine neoplasia type 2B, familial medullary thyroid cancer, pheochromocytoma, paraganglioma), pancreatic cancer (e.g., pancreatic andenocarcinoma, intraductal papillary mucinous neoplasm (IPMN), Islet cell tumors, ductal adenocarcinoma, insulinoma, glucagonoma, vipoma), adrenal gland cancer, neuroendocrine cancer (e.g., gastroenteropancreatic neuroendocrine tumor (GEP-NET), carcinoid tumor), sebaceous gland carcinoma, sweat gland carcinoma). In certain embodiments, the cancer is sweat gland cancer (e.g., sweat gland carcinoma).

In certain embodiments, the cancer is liver cancer (e.g., hepatocellular cancer (HCC) (e.g., hepatocellular carcinoma, hepatoblastoma, hepatocellular adenoma), malignant hepatoma, hemangiomas, biliary cancer (e.g., cholangiocarcinoma)).

In certain embodiments, the cancer is head and neck cancer (e.g., squamous cell carcinoma of the head and neck (SCCHN), adenoid cystic carcinoma). In certain embodiments, the cancer is oral cancer (e.g., buccal cavity cancer, lip cancer, tongue cancer, mouth cancer, pharynx cancer, hypopharynx cancer (e.g., hypopharyngeal carcinoma), throat cancer (e.g., laryngeal cancer, pharyngeal cancer, nasopharyngeal cancer, oropharyngeal cancer), salivary gland cancer). In certain embodiments, the cancer is esophageal cancer (e.g., esophageal squamous cell carcinoma, esophageal adenocarcinoma, Barrett's adenocarcinoma, esophageal leiomyosarcoma).

In certain embodiments, the cancer is gastrointestinal cancer (e.g., anal cancer, colorectal cancer (e.g., colon cancer, rectal cancer, colorectal adenocarcinoma), gall bladder cancer, gastric cancer (e.g., stomach cancer (e.g., stomach adenocarcinoma)), gastrointestinal stromal tumor (GIST), small bowel cancer (e.g., appendix cancer, small bowel carcinoma, e.g., small bowel adenocarcinoma), small intestine cancer, large bowel cancer, large intestine cancer).

In certain embodiments, the cancer is cardiovascular cancer (e.g., primary cardiac tumors, angiosarcoma (e.g., lymphangiosarcoma, lymphangioendotheliosarcoma, hemangiosarcoma), endotheliosarcoma (e.g., Kaposi's sarcoma, multiple idiopathic hemorrhagic sarcoma), cardiac myxoma, cardiac rhabdomyoma).

In certain embodiments, the cancer is lung cancer (e.g., bronchus cancer (e.g., bronchogenic carcinoma, bronchial adenoma), alveolar carcinoma, mesothelioma, small cell lung cancer (SCLC), non-small cell lung cancer (NSCLC), lung adenocarcinoma, chondromatous hamartoma, papillary adenocarcinoma).

In certain embodiments, the cancer is a genitourinary cancer (e.g., bladder cancer (e.g., urothelial carcinoma), urethral cancer, kidney cancer (e.g., nephroblastoma a.k.a. Wilms' tumor, renal cell carcinoma), testicular cancer (e.g., seminoma, testicular embryonal carcinoma), germ cell cancer, prostate cancer (e.g., prostate adenocarcinoma), penile cancer (e.g., Paget's disease of the penis and scrotum)).

In certain embodiments, the cancer is a gynecological cancer (e.g., endometrial cancer (e.g., uterine cancer (e.g., uterine sarcoma, choriocarcinoma), endometrial carcinoma), cervical cancer (e.g., cervical adenocarcinoma), ovarian cancer (e.g., cystadenocarcinoma, ovarian embryonal carcinoma, ovarian adenocarcinoma), germ cell cancer, vulvar cancer (e.g., Paget's disease of the vulva) vaginal cancer, fallopian tube cancer).

In certain embodiments, the cancer is breast cancer (e.g., adenocarcinoma of the breast, papillary carcinoma of the breast, mammary cancer, medullary carcinoma of the breast, triple negative breast cancer, HER-2 positive breast cancer, HER2-negative breast cancer).

In certain embodiments, the cancer is skin cancer (e.g., squamous cell carcinoma (SCC), keratoacanthoma (KA), melanoma, basal cell carcinoma (BCC), dermatofribroma).

In certain embodiments, the cancer is a soft tissue cancer (e.g., intraepithelial neoplasms, epithelial carcinomas, epithelial sarcomas, adenocarcinomas, adenomas, fibrosarcomas, fibromas, liposarcomas, lipomas, myxomas, teratomas).

In certain embodiments, the cancer is a rare cancer. The term “rare cancer” refers to cancers that occur in a relatively small number of patients. Rare cancers include, but are not limited to, sarcomas (e.g., soft tissue sarcoma, liposarcoma, uterine sarcoma, leiomyosarcoma, myxofibrosarcoma, osteosarcoma, angiosarcoma, Ewing's sarcoma, synovial sarcoma, rhabdomyosarcoma, intimal sarcoma), malignant lymphomas, thymic cancer (e.g., thymomas), mesothelioma, gastrointestinal stromal tumors (GISTs), neuroendocrine cancer, eye cancer, brain tumors, bone soft tissue tumors, skin cancer, and germ cell tumors.

In certain embodiments, the cancer is breast cancer, stomach cancer, ovarian cancer, or esophageal cancer. In certain embodiments, the cancer is breast cancer. In certain embodiments, the cancer is stomach cancer. In certain embodiments, the cancer is ovarian cancer. In certain embodiments, the cancer is esophageal cancer. In certain embodiments, the cancer is multiple myeloma, leukemia, lymphoma, or colorectal cancer. In certain embodiments, the cancer is multiple myeloma. In certain embodiments, the cancer is leukemia. In certain embodiments, the cancer is a lymphoma. In certain embodiments, the cancer is colorectal cancer.

In certain embodiments, the cancer is a HER2-positive cancer. In certain embodiments, the cancer is a HER2-positive cancer and the antibody component of the SPAC is an antibody directed against HER2, or an antigen-binding fragment thereof. In certain embodiments, the cancer is a HER2-positive cancer; the antibody component of the SPAC is an antibody directed against HER2, or an antigen-binding fragment thereof, and the stapled peptide is a stapled anti-cancer peptide. In certain embodiments, the HER2-positive cancer is breast cancer, stomach cancer, ovarian cancer, or esophageal cancer. In certain embodiments, the HER2-positive cancer is HER2-positive breast cancer. In certain embodiments, the antibody directed against HER2 is trastuzumab or pertuzumab. Other examples of antibodies directed against HER2 are provided herein.

In certain embodiments, the cancer expresses an antigen selected from CD38, CD33, CD22, TROP2, CD30, CD79b, and Nectin-4. In certain embodiments, the cancer expresses said antigen and the antibody component of the SPAC is an antibody directed against said antigen, or an antigen-binding fragment thereof. In certain embodiments, the cancer expresses said antigen; the antibody component of the SPAC is an antibody directed against said antigen, or an antigen-binding fragment thereof; and the stapled peptide is a stapled anti-cancer peptide.

In certain embodiments, the cancer expresses CD38. In certain embodiments, the cancer expresses CD38 and the antibody component of the SPAC is an antibody directed against CD38, or an antigen-binding fragment thereof. In certain embodiments, the cancer expresses CD38; the antibody component of the SPAC is an antibody directed against CD38, or an antigen-binding fragment thereof; and the stapled peptide is a stapled anti-cancer peptide. In certain embodiments, the cancer expressing CD38 is multiple myeloma, leukemia, lymphoma, or colorectal cancer. In certain embodiments, the antibody directed against HER2 is daratumumab. Other examples of antibodies directed against CD38 are provided herein.

Additionally, provided herein are methods of inhibiting tumor growth in a subject comprising administering to the subject an effective amount of a SPAC provided herein, or a pharmaceutical composition thereof. Also provided herein are SPACs, and pharmaceutical compositions thereof, for use in inhibiting tumor growth in a subject. Also provided herein are uses of SPACs provided herein, and pharmaceutical compositions thereof, for the manufacture of medicaments for inhibiting tumor growth. The tumor may express any of the antigens described herein (e.g., HER2, CD38, CD33, CD22, TROP2, CD30, CD79b, Nectin-4).

As used herein the term “inhibit” or “inhibition” in the context of tumor growth, for example, refers to a reduction in the rate of growth of the tumor (i.e., reduction in the rate of proliferation of the tumor's cells). In some embodiments, the term refers to a reduction in the rate of tumor growth to a level that is statistically significantly lower than an initial rate (e.g., the rate of tumor growth before administration or application of a SPAC provided herein). In some embodiments, the term refers to a reduction in the rate of tumor growth to a rate that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial rate (e.g., the rate of tumor growth before administration or application of a SPAC provided herein).

In certain embodiments, treating cancer and/or inhibiting tumor growth can result in a reduction in size or volume of a tumor. For example, after treatment, tumor size is reduced by 5% or greater (e.g., 10%, 20%, 30%, 40%, 50%, 60%, 70%, 80%, 90% or greater) relative to its size prior to treatment. Size of a tumor may be measured by any reproducible means of measurement. The size of a tumor may be measured as a diameter of the tumor or by any reproducible means of measurement. In certain embodiments, the tumor size is reduced by at least 25% relative to its size prior to treatment.

In certain embodiments, treating cancer and/or inhibiting tumor growth may further result in a decrease in number of tumors. For example, after treatment, tumor number is reduced by 5% or greater (e.g., 10%, 20%, 30%, 40%, 50%, 60%, 70%, 80%, 90% or greater) relative to number prior to treatment. Number of tumors may be measured by any reproducible means of measurement. The number of tumors may be measured by counting tumors visible to the naked eye or at a specified magnification (e.g., 2×, 3×, 4×, 5×, 10×, or 50×).

In certain embodiments, treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. The number of metastatic nodules may be measured by counting metastatic nodules visible to the naked eye or at a specified magnification (e.g., 2×, 10×, or 50×).

In certain embodiments, treating cancer can result in an increase in average survival time of a population of subjects treated according to the present disclosure in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the present disclosure. An increase in average survival time of a population may also be measured, for example, by calculating for a population the average length of survival following completion of a first round of treatment with the compound of the present disclosure.

In certain embodiments, treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with the compound of the present disclosure. A decrease in the mortality rate of a population may also be measured, for example, by calculating for a population the average number of disease-related deaths per unit time following completion of a first round of treatment with the compound of the present disclosure.

In certain embodiments, treating cancer can also result in an increased average progression-free survival time of a population of treated subjects in comparison to an untreated population. For example, the average progression-free survival time is increased by more than 30 days (more than 60 days, 90 days, or 120 days). An increase in average progression-free survival time of a population may be measured by any reproducible means. An increase in average progression-free survival time of a population may be measured, for example, by calculating for a population the average length of progression-free survival following initiation of treatment with the compound of the present disclosure. An increase in average progression-free survival time of a population may also be measured, for example, by calculating for a population the average length of progression-free survival following completion of a first round of treatment with the compound of the present disclosure. “Progression-free survival” as used herein refers to the length of time during and after medication or treatment during which the disease being treated (e.g., cancer) does not get worse.

Also provided herein are methods of delivering a stapled peptide into a cell comprising contacting the cell with a SPAC provided herein, or a pharmaceutical composition thereof. In certain embodiments, the cell is a cancer cell. In certain embodiments, the stapled peptide has improved cellular uptake relative to a corresponding unconjugated stapled peptide. In certain embodiments, the stapled peptide in delivered to the cell in vitro. In certain embodiments, the stapled peptide is delivered to the cell in vivo (i.e., in a subject). In certain embodiments, the stapled peptide is delivered to the cells of a biological sample.

Also provided herein are methods of triggering cancer cell death comprising contacting the cancer cell with an effective amount of a SPAC provided herein, or a pharmaceutical composition thereof. In certain embodiments, the method is a method for selectively triggering cancer cell death (i.e., selectively killing cancer cells). In certain embodiments, a SPAC provided herein is selectively cytotoxic to cancer cells. In certain embodiments, the cell is contacted in vitro. In certain embodiments, the cell is contacted in vivo (i.e., in a subject). In certain embodiments, the cell is contacted in a biological sample.

A SPAC described herein “selectively” triggers the death of one type of cell over another (e.g., selectively triggers cancer cell death over non-cancer cell death) if it triggers cell death of one type of cell to a greater extent than the other. A SPAC described herein “selectively” triggers cancer cell death if it triggers cancer cell death to a greater extent than non-cancer cell death. A peptide described herein is “selectively” cytotoxic to cancer cells over non-cancer cells if it is toxic (e.g., by lysing, killing, promoting apoptosis of, or otherwise damaging) to cancer cells to a greater extent than the non-cancer cells. In certain embodiments, the selectivity in any of the foregoing embodiments is at least 1.1-fold, at least 1.5-fold, 2-fold, at least 3-fold, at least 5-fold, at least 10-fold, at least 30-fold, at least 50-fold, at least 100-fold, at least 300-fold, at least 500-fold, at least 1,000-fold, at least 3,000-fold, at least 5,000-fold, at least 10,000-fold, at least 30,000-fold, at least 50,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure.

As described herein, the cells may be cancer cells. In certain embodiments, the cells are breast cancer (e.g., BT-474, SK-BR-3, MCF-7), ovarian cancer (e.g., SK—OV-3), esophageal cancer (e.g., OE19, KYSE-410), stomach cancer (e.g., NCI-N87), multiple myeloma (e.g., NCIH929, RMPI 8226), colorectal cancer (e.g., HCT-116, COLO-678), lymphoma: (e.g., DAUDI) or ALL (e.g., DND41) cells.

Also provided herein are methods of treating and/or preventing an infectious disease (e.g., bacterial infection) in a subject comprising administering to the subject an effective amount of a SPAC provided herein, or a pharmaceutical composition thereof. Also provided herein are SPACs, and pharmaceutical compositions thereof, for use in treating and/or preventing an infectious disease (e.g., bacterial infection) in a subject. Also provided herein are uses of SPACs, and pharmaceutical compositions thereof, for the manufacture of medicaments for treating and/or preventing infectious diseases (e.g., bacterial infections). In certain embodiments, the infectious disease is a bacterial infection. In certain embodiments, the bacterial infection is an antibiotic-resistant bacterial infection.

Also provided herein are method of killing and/or inhibiting the growth of bacteria (e.g., Gram-negative and/or Gram-positive bacteria) comprising contacting the bacteria (e.g., in vitro or in vivo) with a SPAC provided herein, or a pharmaceutical composition thereof.

An “infectious disease” refers to any disease caused by a pathogen (i.e., pathogenic microorganisms). An infectious disease may be caused by bacteria, viruses, parasites, or fungi. An infectious disease can be a microbial infection. A “microbial infection” refers to an infection with a microorganism, such as a fungus, bacteria or virus. Various bacterial infections include, but are not limited to, skin infections (e.g., bacterial cellulitis, wound infections), gastrointestinal infections, throat infections (e.g., strep throat), urinary tract infections (UTIs), genito-urinary infections, sexually-transmitted diseases (e.g., gonorrhea, chlamydia, syphilis), pulmonary infections (e.g., pneumonia, pneumococcal pneumonia, tuberculosis, whooping cough), food poisoning, sepsis, bacterial meningitis, Lyme disease, cholera, botulism, tetanus, anthrax, blood infections, and systemic infections. In certain embodiments, the microbial infection is an infection with bacteria, i.e., a “bacterial infection.” In certain embodiments, the bacteria are Gram-negative bacteria. In certain embodiments, the bacteria are Gram-positive bacteria.

“Gram-negative bacteria” were first defined by their ability not to retain Gram staining; however, since then Gram-negative bacteria have been further defined as bacteria generally having a cell wall with a thin peptidoglycan layer and have an outer lipid membrane. “Gram-positive bacteria” are bacteria that take up the crystal violet color in the Gram staining test, and generally have cell walls comprising a thick peptidoglycan layer and no outer lipid membrane.

As used herein, an “antibiotic-resistant bacterial infection” is a bacterial infection caused by antibiotic-resistant bacteria. “Antibiotic resistance” occurs when bacteria evolve mechanisms that protect them from the effects of antibiotics. Microbes resistant to multiple antimicrobials are referred to as “multidrug resistant” (MDR). In certain embodiments, methods herein are for treating multidrug resistant bacterial infections. In certain embodiments, methods herein are for killing and/or inhibiting the growth of multidrug resistant bacteria.

### EXAMPLES

### General Methods

Solid phase peptide synthesis: Fmoc-based solid-phase peptide synthesis was used to synthesize the antimicrobial peptides and their stapled derivatives. To achieve the i+4 staple lengths, α-methyl, α-alkenyl amino acids were used flanking three residues. For the stapling reaction, Grubbs 1st generation ruthenium catalyst dissolved in dichloroethane was added to the peptides while still on resin. To ensure maximal conversion, three to five rounds of stapling were performed. Once stapled, the appropriate maleimide linker was coupled to the N-terminus and the linker-peptides were cleaved off the resin using trifluoroacetic acid, then precipitated using a hexane:ether (1:1) mixture, and afterwards purified using a prep HPLC. Final linker-peptide characterization for purity was assessed using a UHPLC/MS system.

Cell culture: Cell lines were maintained in appropriate medium supplemented with fetal bovine serum to a final concentration of 10%.

72 hour cytotoxicity assay: Cells were plated in a 96-well format, and after 24 hour incubation, serial dilutions of SPACs from a 1 mg/mL stock, or vehicle, were then added to the cells in a final volume of 100 μl. After incubating at 37° C. for 72 hours, 100 μl of CellTiter-Glo® reagent was added to the cells, and the plates were incubated 15 minutes at room temperature. Luminescence was then measured on a microplate reader.

### Example 1: Preparation of SPACs

Peptide conjugation protocol: (1) Prepare stapled peptide using solid-phase synthesis; (2) On resin, to an N-terminally deprotected stapled peptide, add a linking reagent. For example, 6-maleimidohexanoyl-Val-Cit-p-aminobenzoylcarbonate-4-nitrophenyl ester (Mc-Val-Cit-PABC-PNP) can be used; (3) Cleave from resin and purify by HPLC; (4) In a separate batch, reduce the antibody using TCEP (see protocol below); (5) Conjugate the stapled peptide to the antibody; (6) Quench unreacted peptide using N-acetylcysteine; and (7) Purify by salt exchange.

Antibody conjugation protocol: (1) Buffer exchange the antibody into PBS-E, pH 6-8, by the following protocol: (a) Dilute a 21 mg/mL solution of antibody provided by the manufacturer to 1 mg/mL in PBS-E, pH 6-8, (b) Remove the loading buffer by centrifugating using a spin column. Add more PBS-E and equilibrate by centrifugating further, (c) Add antibody solution (1 mg/mL), centrifugate, collecting the flowthrough, (d) Measure the antibody concentration in the eluate, and (e) Use an ultrafiltration device per manufacturer instructions to raise antibody concentration to 10 mg/mL; (2) Reduce antibody with TCEP; (3) Conjugate peptide through co-incubation at room temperature; (4) Quench reaction with n-acetylcysteine; and (5) Purify.

### Example 2: Cytotoxicity of Anti-HER2 SPACs

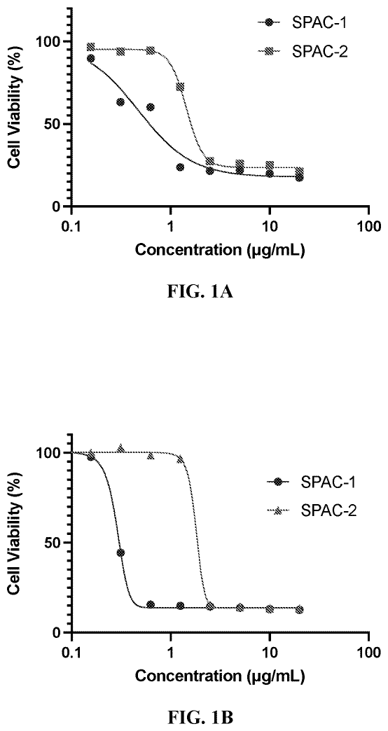

Anti-HER2 SPAC 1 and SPAC 2 (see Table 6) are cytotoxic to breast cancer cells with both relatively low and relatively high HER2 expression. See FIGS. 1A-1B. SPAC 1 and SPAC 2 showed cytotoxicity in BT-474 cell line with high HER2 expression (HER2+++) (FIG. 1A). SPAC 1 and SPAC 2 also show cytotoxicity in MCF7 cell line with low HER2 expression (HER2+) (FIG. 1B).

### Example 3: Cytotoxicity of Anti-CD38 SPACs

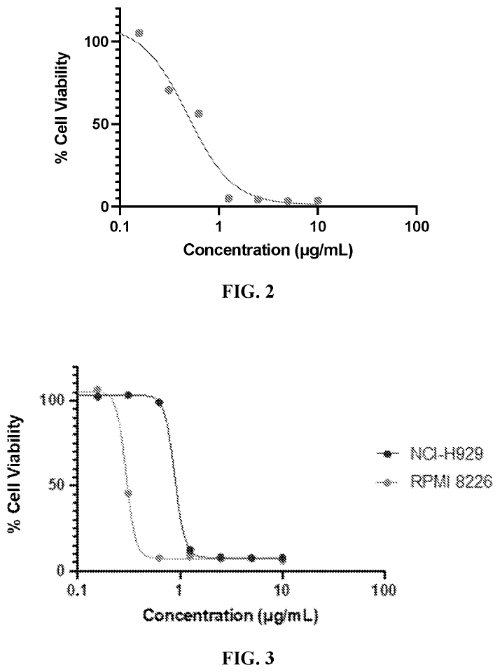

Anti-CD38 SPAC 3 (see Table 6) is cytotoxic to CD38+ multiple myeloma cells. See FIG. 2. SPAC 3 shows cytotoxicity in a CD38+ multiple myeloma cell line, RPMI 8226.

### Example 4: Antiproliferative Activity of Anti-CD38 SPACs Comprising PPI Inhibitors

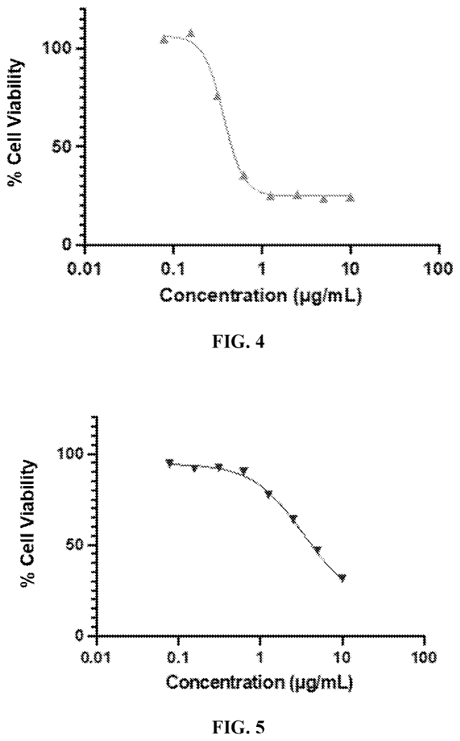

Anti-CD38 SPACs were used to deliver a variety of stapled peptide protein-protein interaction (PPI) inhibitors (e.g., MCL-1 inhibitors, MDM2 inhibitors, β-catenin inhibitors) to inhibit proliferation of cancer cells. As shown in FIGS. 3-5, SPACs 8, 11, and 16(see Table 7), each comprising the anti-CD38 antibody daratumumab, inhibit the proliferation of multiple myeloma cells.

### Example 5: Selectivity of Anti-HER2 SPACs Comprising PPI Inhibitors

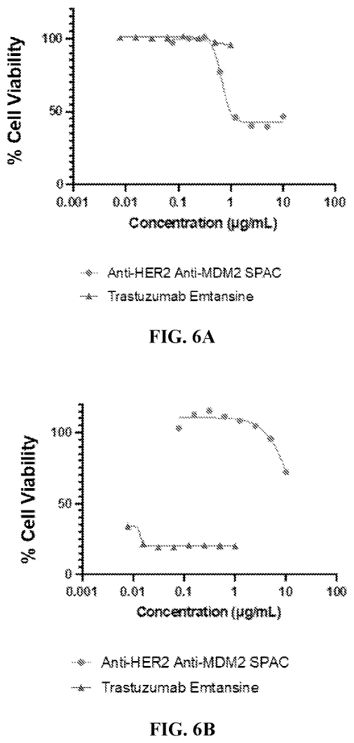

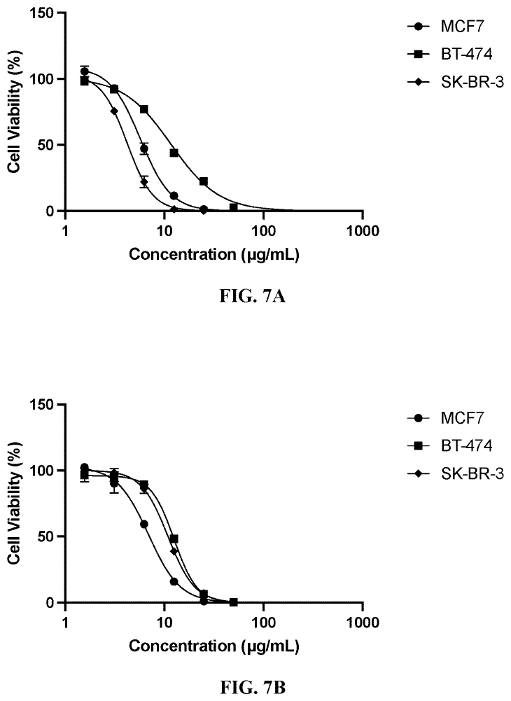

As shown in FIGS. 6A-6B, SPACs can be highly selective, which is beneficial when trying to avoid on-target toxicity associated with traditional ADCs. SPACs can also overcome low receptor expression by taking advantage of oncogene addiction. FIG. 6A shows results in a HER2-low cell line, MCF7, which has wild-type (wt) p53 status and is thus sensitive to MDM2 inhibition. Trastuzumab emtansine, a traditional ADC, shows no activity in this cell line, while SPAC 7 provided herein (see Table 7) comprising an MDM2 inhibitor shows anti-proliferative activity. FIG. 6B shows results in a cell line that highly expresses HER2 but has a mutated p53 and thus is insensitive to MDM2 inhibition. In this setting, the traditional ADC trastuzumab emtansine shows efficacy at relatively low concentration while SPAC 7 (see Table 7) requires a relatively high concentration. These data demonstrate the selectivity of SPACs. These data also demonstrate that on-target toxicity is less likely since for toxicity, one may need high antibody concentration and receptor expression that is higher than in normal tissue.

### ADDITIONAL EMBODIMENTS

Additional embodiments of the disclosure are indicated by the following numbered paragraphs:

1. A stapled peptide-antibody conjugate (SPAC) comprising a stapled peptide conjugated to an antibody or antigen-binding fragment thereof.

2. The SPAC of paragraph 1, wherein the antibody is a monoclonal antibody (mAb) or antigen-binding fragment thereof.

3. The SPAC of paragraph 1 or 2, wherein the antibody is an anti-cancer antibody or antigen-binding fragment thereof.

4. The SPAC of any one of paragraphs 1-3, wherein the antibody or antigen-binding fragment thereof is directed to a target antigen expressed on a cancer cell.

5. The SPAC of any one of paragraphs 1-4, wherein the antibody is an antibody directed against human epidermal growth factor receptor 2 (HER2), or an antigen-binding fragment thereof.

6. The SPAC of paragraph 5, wherein the antibody is trastuzumab.

7. The SPAC of paragraph 5, wherein the antibody is pertuzumab.

8. The SPAC of any one of paragraphs 1-4, wherein the antibody is an antibody directed against CD38, CD33, CD22, TROP2, CD30, CD79b, Nectin-4, or TM4SF1, or antigen-binding fragment thereof.

9. The SPAC of any one of paragraphs 1-4, wherein the antibody is an antibody directed against TM4SF1, or an antigen-binding fragment thereof.

10. The SPAC of any one of paragraphs 1-4, wherein the antibody is an antibody directed against CD38, or an antigen-binding fragment thereof.

11. The SPAC of paragraph 10, wherein the antibody daratumumab.

12. The SPAC of any one of paragraph 1-11, wherein the antibody is an antibody-drug conjugate (ADC) or an antigen-binding fragment thereof.

13. The SPAC of paragraph 12, wherein the antibody is trastuzumab emtansine.

14. The SPAC of any one of paragraphs 1-13, wherein the stapled peptide is a stapled anti-cancer peptide.

15. The SPAC of any one of paragraphs 1-14, wherein the stapled peptide is a stapled antimicrobial peptide (StAMP).

16. The SPAC of any one of paragraphs 1-15, wherein the stapled peptide is a singly stapled, doubly stapled, or stitched peptide.

17. The SPAC of any one of paragraphs 1-16, wherein the stapled peptide is an inhibitor of a protein-protein interaction (PPI).

18. The SPAC of any one of paragraphs 1-17, wherein the stapled peptide is an inhibitor of a BCL-2 family member protein.

19. The SPAC of paragraph 18, wherein the stapled peptide is an inhibitor of BCL-x_{L}, BCL-2, BCL-W, and/or MCL1.

20. The SPAC of paragraph 19, wherein the stapled peptide is an MCL1 inhibitor.

21. The SPAC of any one of paragraphs 1-20, wherein the stapled peptide is an activator of a BCL-2 family member protein effector.

22. The SPAC of paragraph 21, wherein the stapled peptide is an activator of BAX, BAK, or BOK.

23. The SPAC of any one of paragraphs 1-22, wherein the stapled peptide is a stapled BCL-2-interacting mediator of cell death (BIM) peptide.

24. The SPAC of any one of paragraphs 1-23, wherein the stapled peptide is SAHB_{A1} or BIM SAHB_{A2}.

25. The SPAC of any one of paragraphs 1-17, wherein the stapled peptide is a 3-catenin inhibitor.

26. The SPAC of paragraph 25, wherein the stapled peptide is an inhibitor of Wnt/0-catenin signaling.

27. The SPAC of any one of paragraphs 1-17, wherein the stapled peptide is an MDM2 inhibitor.

28. The SPAC of paragraph 27, wherein the stapled peptide inhibits the binding of MDM2 to p53.

29. The SPAC of paragraph 27 or 28, wherein the stapled peptide is ALRN-6924, ATSP-7041 or ATSP 7041 Cba10L.

30. The SPAC of paragraph 27 or 28, wherein the stapled peptide comprises any one of SEQ ID NOs: 161-166, or a pharmaceutically acceptable salt thereof.

31. The SPAC of any one of paragraphs 1-30, wherein the stapled peptide triggers cancer cell death.

32. The SPAC of any one of paragraphs 1-17, wherein the stapled peptide is a stapled Magainin peptide.

33. The SPAC of paragraph 32, wherein the stapled peptide is a stapled Magainin II peptide.

34. The SPAC of paragraph 33, wherein the stapled peptide comprises the amino acid sequence:

(SEQ ID NO: 1)
G X¹ G K F X² H S K K K F G K A X³ V G E X⁴,

or a pharmaceutically acceptable salt thereof, wherein:
X¹, X², X³, and X⁴ are amino acids;
X¹ and X² are connected via a crosslink, and X³ and X⁴ are connected via a crosslink; and
the amino acid sequence includes 0 to 9 amino acid substitutions, inclusive, at positions other than X¹, X², X³, and X⁴.

35. The SPAC of paragraph 34, wherein the stapled peptide comprises the amino acid sequence:

(SEQ ID NO: 2)
G X¹ G K F X² H S K K K F G K A X³ V G E X⁴ A K K,

or a pharmaceutically acceptable salt thereof, wherein the amino acid sequence includes 0 to 11 amino acid substitutions, inclusive, at positions other than X¹, X², X³, and X⁴.

36. The SPAC of paragraph 35, wherein the stapled peptide comprises the amino acid sequence:

(SEQ ID NO: 48)
G X¹ G K F X² Dap K K K K F G K A X³ V G E X⁴ A K
K,

or a pharmaceutically acceptable salt thereof.

37. The SPAC of paragraph 35, wherein the stapled peptide comprises the amino acid sequence:

(SEQ ID NO: 26)
G X¹ G Dap F X² Dap Dap Dap Dap Dap F G Dap A X³ V
GE X⁴ A Dap Dap,
(SEQ ID NO: 5)
G X¹ G K F X² K K K K K F G K A X³ V G E X⁴ K K,
(SEQ ID NO: 23)
G X¹ G K F X² H K K K K F G K A X³ V F E X⁴ A K K,
(SEQ ID NO: 24)
G X¹ G Dab F X² Dab Dab Dab Dab Dab FG Dab A X³ V
G E X⁴ A Dab Dab,

or a pharmaceutically acceptable salt thereof.

38. The SPAC of any one of paragraphs 1-17, wherein the stapled peptide is a stapled Esculentin peptide.

39. The SPAC of paragraph 38, wherein the stapled peptide is a stapled esculentin-1A peptide.

40. The SPAC of paragraph 39, wherein the stapled peptide comprises the amino acid sequence:

(SEQ ID NO: 73)
G X¹ F S K X² K G K K I K N L X³ I S G X⁴ K G,

and pharmaceutically acceptable salts thereof, wherein:
X¹, X², X³, and X⁴ are amino acids;
X¹ and X² are connected via a crosslink, and X³ and X⁴ are connected via a crosslink; and
the amino acid sequence includes 0 to 9 amino acid substitutions, inclusive, at positions other than X¹, X², X³, and X⁴.

41. The SPAC of paragraph 40, wherein the stapled peptide comprises the amino acid sequence:

(SEQ ID NO: 105)
G X¹ F S K X² K G K K I K N L L X³ S G L X⁴ G,

and pharmaceutically acceptable salts thereof, wherein:
X¹, X², X³, and X⁴ are amino acids;
X¹ and X² are connected via a crosslink, and X³ and X⁴ are connected via a crosslink; and
the amino acid sequence optionally includes 0 to 8 amino acid substitutions, inclusive, at positions other than X¹, X², X³, and X⁴.

42. The SPAC of paragraph 40, wherein the stapled peptide comprises SEQ ID NO: 73, or a pharmaceutically acceptable salt thereof.

43. The SPAC of paragraph 41, wherein the stapled peptide comprises SEQ ID NO: 105, or a pharmaceutically acceptable salt thereof.

44. The SPAC of paragraph 41, wherein the stapled peptide comprises the amino acid sequence:

(SEQ ID NO: 113)
G X¹ F S K X² K G K K I K N L L X³ S G L X⁴ K G G
E,

or a pharmaceutically acceptable salt thereof.

45. The SPAC of any one of paragraphs 1-44, wherein the stapled peptide is an α-helical peptide.

46. The SPAC of any one of paragraphs 1-45, wherein the stapled peptide is 100 amino acids or fewer in length.

47. The SPAC of any one of paragraphs 1-46, wherein the stapled peptide is 30 amino acids or fewer in length.

48. The SPAC of any one of paragraphs 1-47, wherein the stapled peptide is conjugated to one or more stapled peptides.

49. The SPAC of paragraph 36, wherein the stapled peptide is of SEQ ID NO: 48, wherein X¹ and X², and X³ and X⁴, are connected via a crosslink of the following formula:

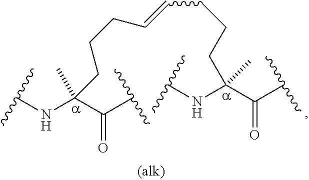 [SMILES: *N[C@@](C)(CCCC=CCCC[C@](C)(N*)C(*)=O)C(*)=O]

wherein α denotes the α-carbons of the crosslinked amino acids; and wherein the C-terminus of the stapled peptide is amidated with —NH₂.

50. The SPAC of paragraph 37, wherein the stapled peptide is of SEQ ID NO: 26, wherein X¹ and X², and X³ and X⁴, are connected via a crosslink of the following formula (alk); and wherein the C-terminus of the stapled peptide is amidated with —NH₂.

51. The SPAC of paragraph 42, wherein the stapled peptide is of SEQ ID NO: 73, wherein X¹ and X², and X³ and X⁴, are connected via a crosslink of the following formula (alk); and wherein the C-terminus of the stapled peptide is amidated with —NH₂.

52. The SPAC of paragraph 43, wherein the stapled peptide is of SEQ ID NO: 105, wherein X¹ and X², and X³ and X⁴, are connected via a crosslink of the following formula (alk); and wherein the C-terminus of the stapled peptide is amidated with —NH₂.

53. The SPAC of paragraph 44, wherein the stapled peptide is of SEQ ID NO: 113, wherein X¹ and X², and X³ and X⁴, are connected via a crosslink of the following formula (alk); and wherein the C-terminus of the stapled peptide is amidated with —NH₂.

54. The SPAC of any one of paragraphs 1-53, wherein the antibody or antigen-binding fragment thereof is conjugated to the N-terminus of the stapled peptide.

55. The SPAC of any one of paragraphs 1-53, wherein the antibody or antigen-binding fragment thereof is conjugated to the C-terminus of the stapled peptide via a lysine residue.

56. The SPAC of any one of paragraphs 1-55, wherein the stapled peptide is conjugated to a cysteine residue of the antibody or antigen-binding fragment thereof.

57. The SPAC of any one of paragraphs 1-55, wherein the stapled peptide is conjugated through a lysine residue of the antibody or antigen-binding fragment thereof.

58. The SPAC of any one of paragraphs 1-57, wherein the antibody or antigen-binding fragment thereof is conjugated to the stapled peptide via a linker.

59. The conjugate of paragraph 58, wherein the linker comprises optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof.

60. The SPAC of paragraph 58 or 59, wherein the linker is a cleavable linker.

61. The SPAC of any one of paragraphs 58-60, wherein the linker is pH cleavable or cleavable by a protease, esterase, or intracellular disulfide reduction.

62. The SPAC of paragraph 61, wherein the linker is cleavable by a protease.

63. The SPAC of any one of paragraphs 58-62, wherein the linker is a peptidic linker.

64. The SPAC of paragraph 63, wherein the peptidic linker comprises —Y^{A}Y^{B}Y^{C}Y^{D}—(SEQ ID NO: 159), wherein Y^{A} is glycine, glutamic acid, or is absent; Y^{B} is valine, phenylalanine, alanine, tyrosine, or glycine; Y^{C} is citrulline, arginine, lysine, alanine, or glycine; and Y^{D} is glycine or is absent.

65. The SPAC of paragraph 64, wherein the peptidic linker comprises -valine-citrulline-.

66. The SPAC any one of paragraphs 58-65, wherein the linker is of one of the following formulae:

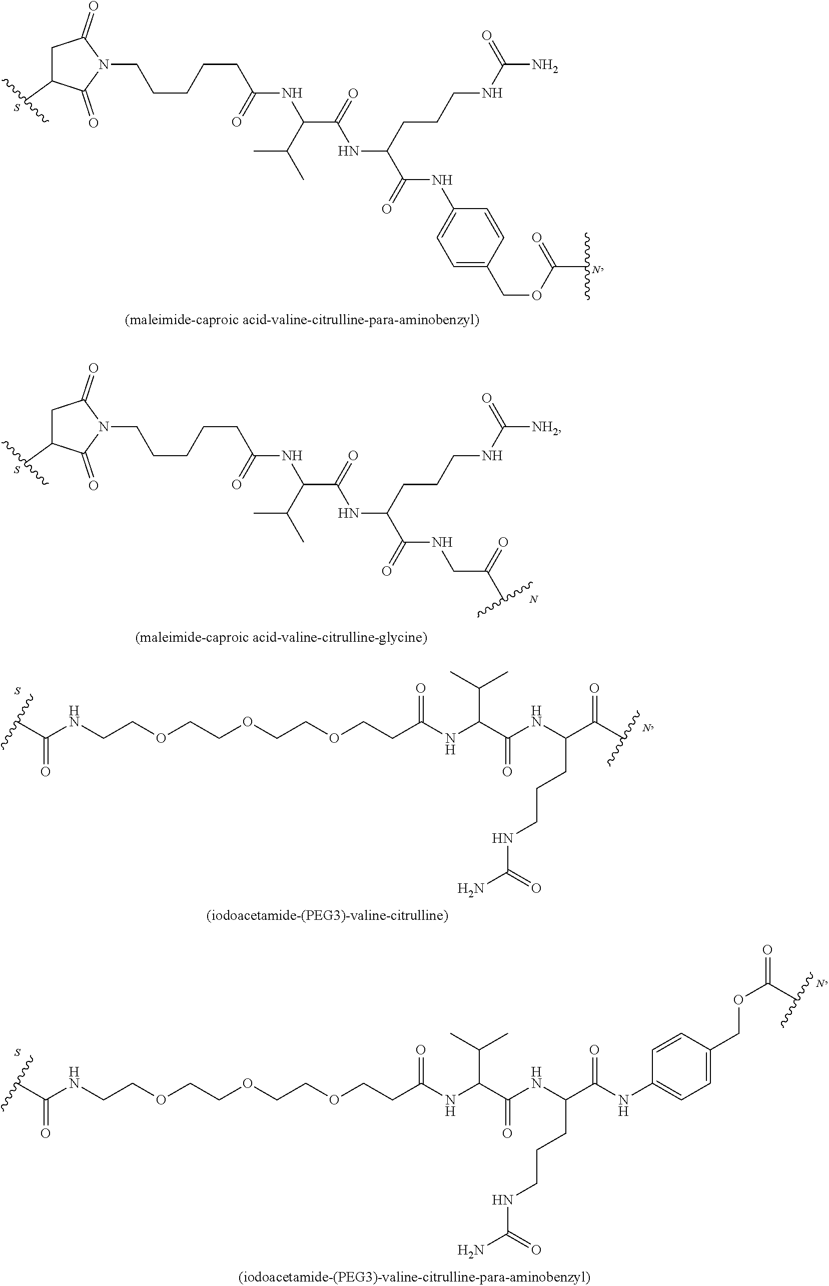

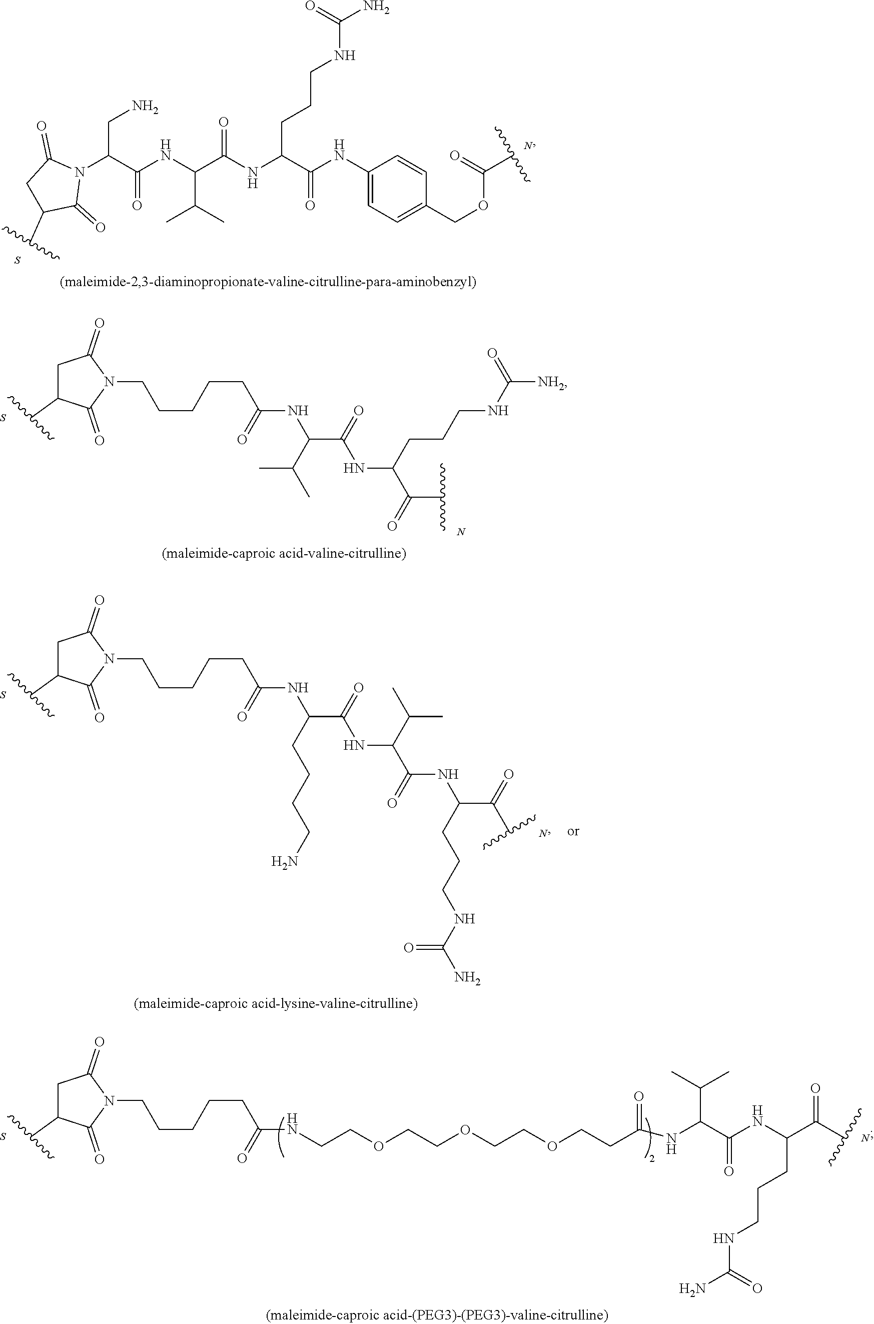

wherein S denotes the point of attachment to the antibody or antigen-binding fragment thereof; N denotes the point of attachment to the N-terminus of the stapled peptide.

67. The SPAC any one of paragraphs 58-65, wherein the linker comprises one of the following formulae:

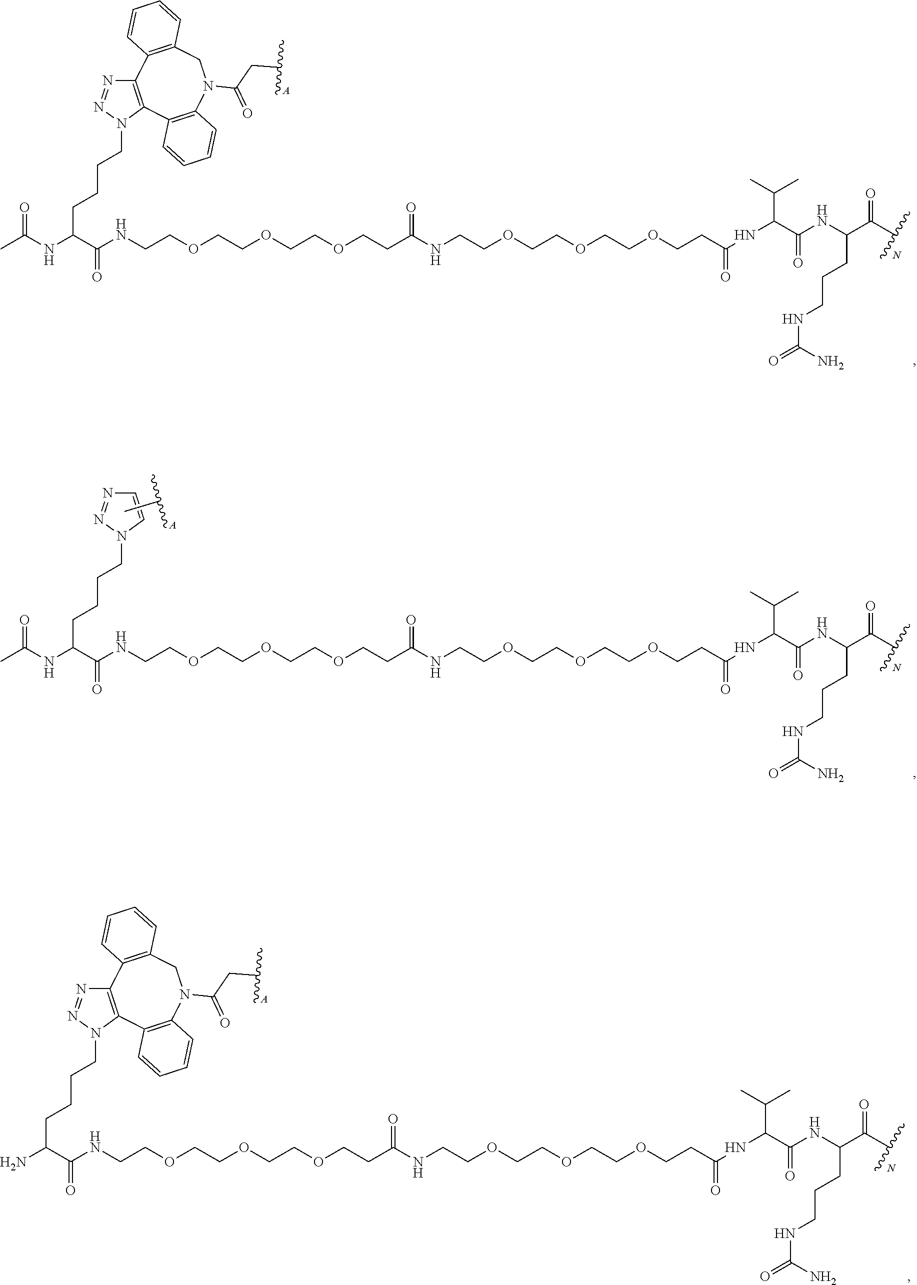

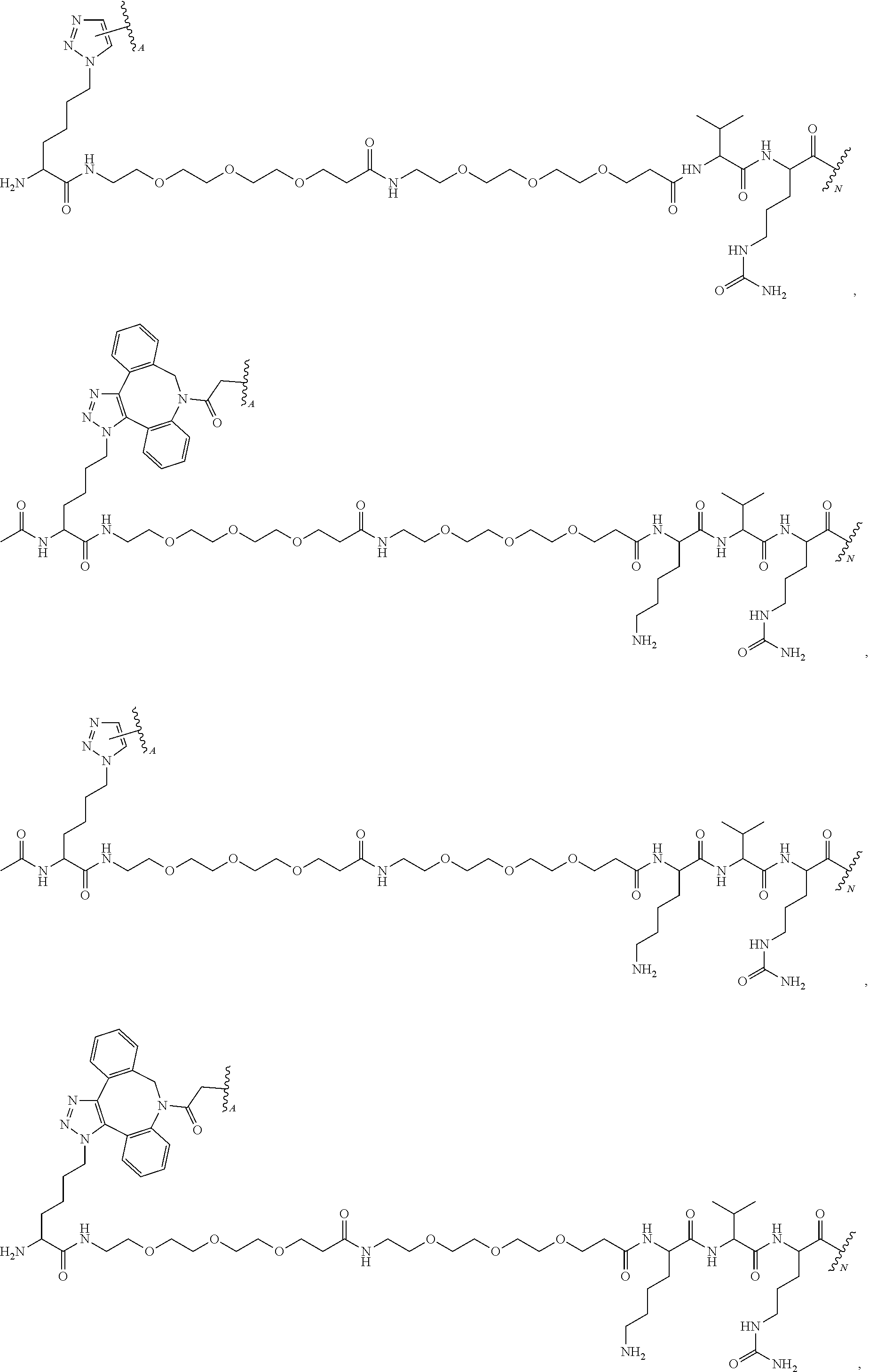

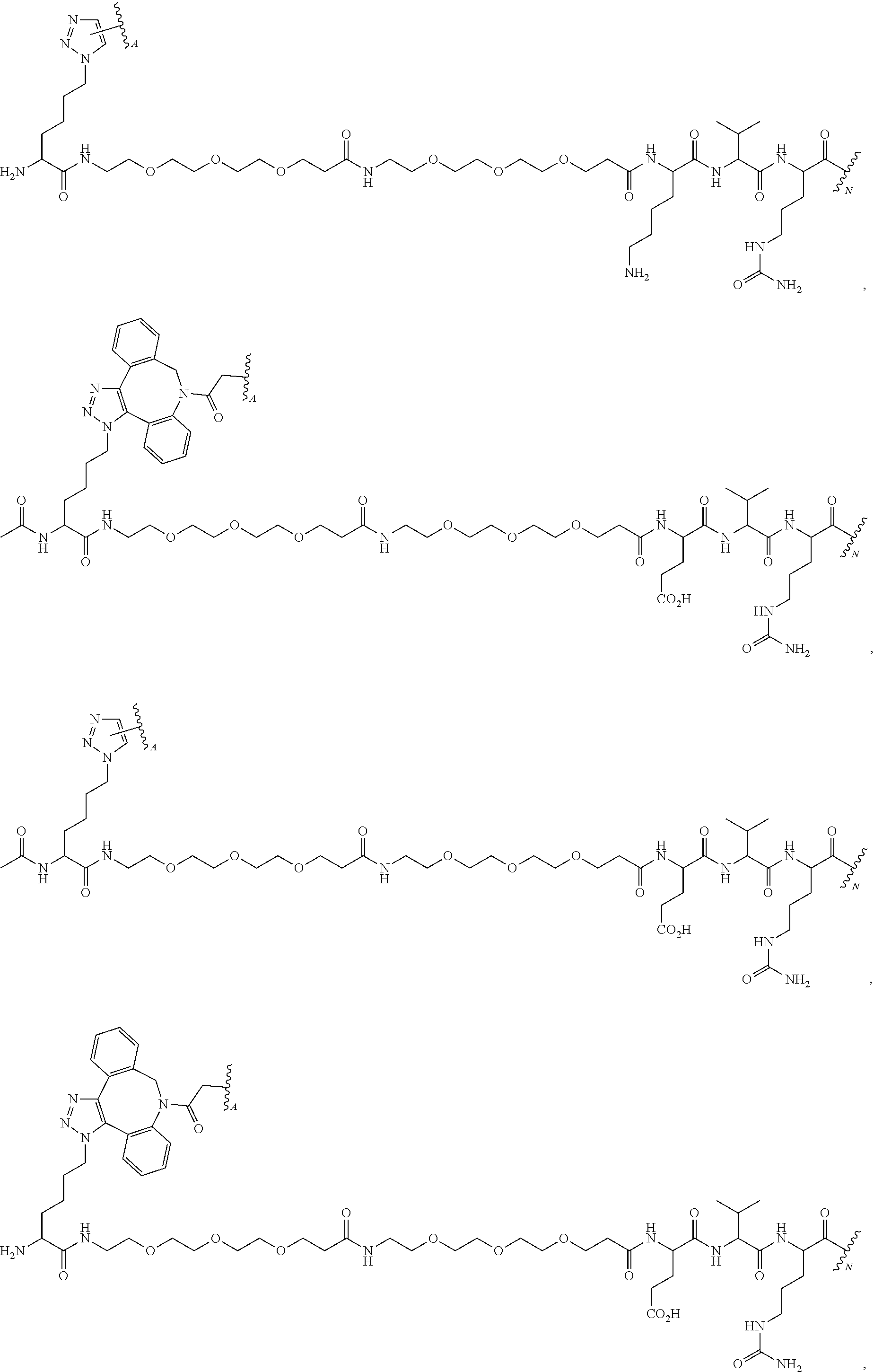

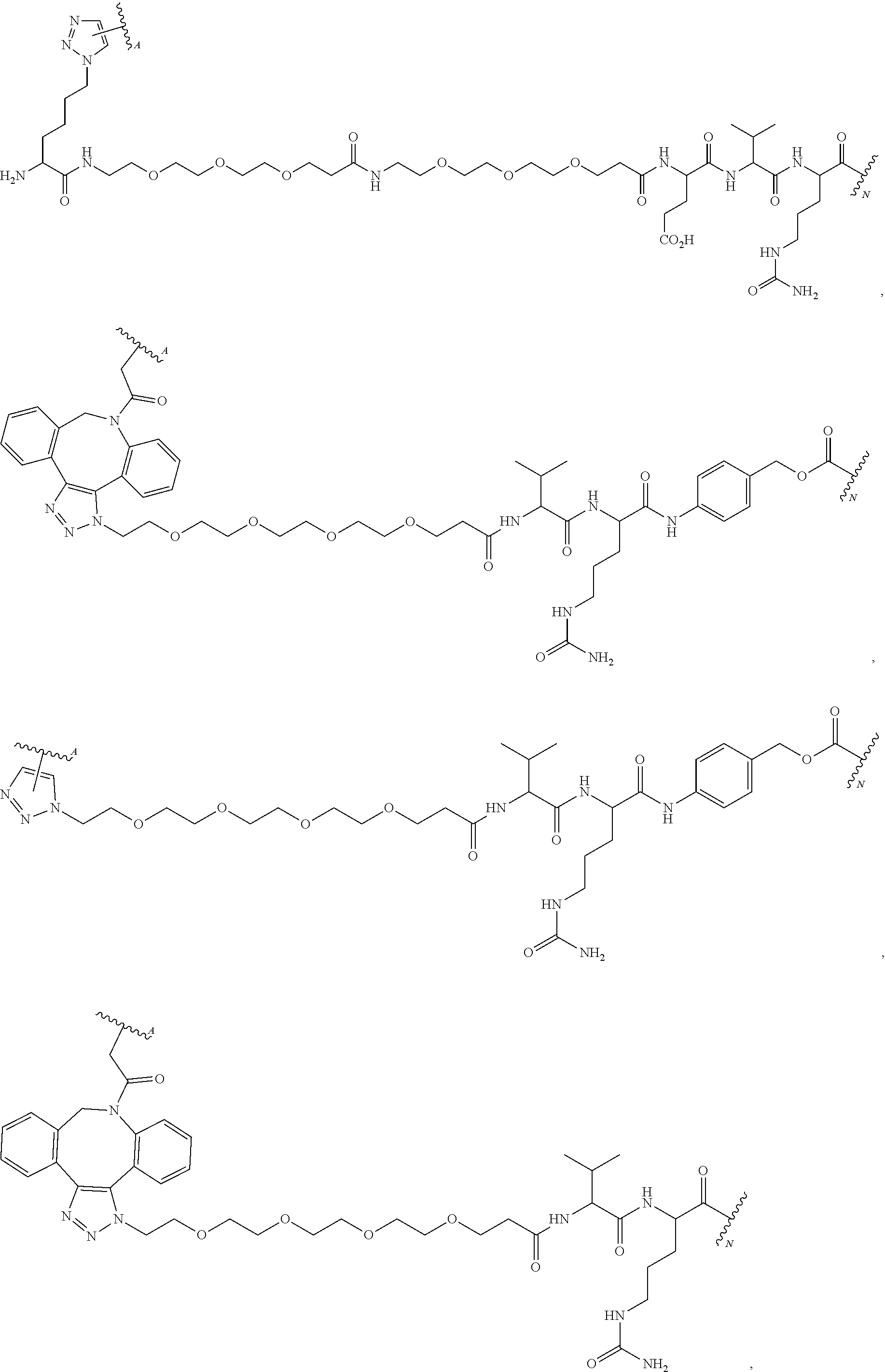

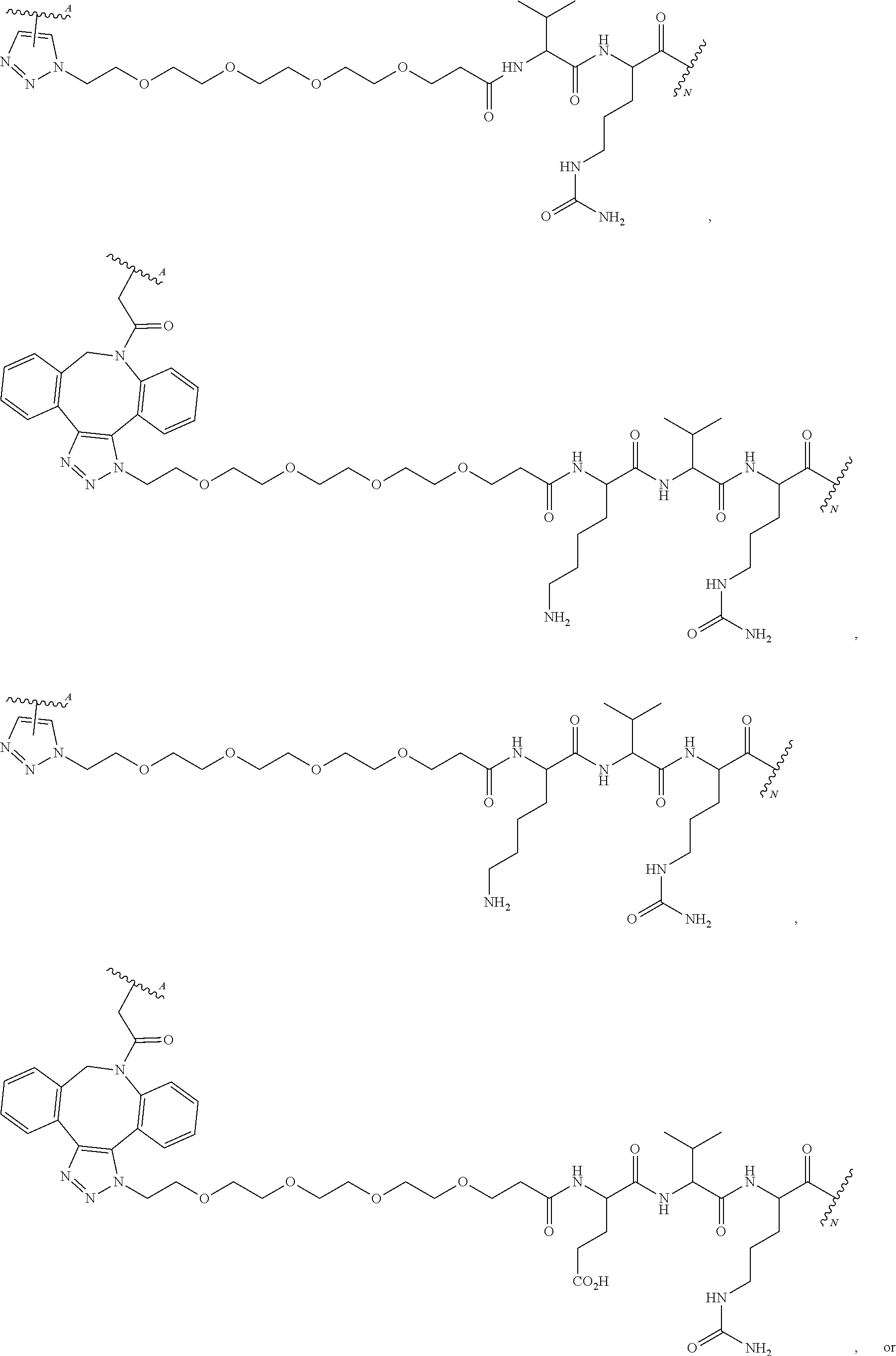

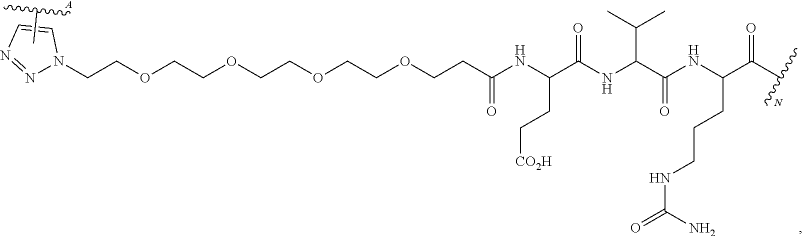

wherein A denotes a point of linkage to the antibody or antigen-binding fragment thereof; N denotes the point of attachment to the N-terminus of the stapled peptide

68. The SPAC of any one of paragraphs 1-67, wherein the antibody or antigen-binding fragment thereof is conjugated to 2 or more stapled peptides.

69. The SPAC of any one of paragraphs 1-68, wherein the antibody or antigen-binding fragment thereof is conjugated to 2-10 stapled peptides, include.

70. The SPAC of any one of paragraphs 1-69 comprising an antibody or antigen-binding fragment thereof to stapled peptide ratio of about 1:8.

71. A pharmaceutical composition comprising an SPAC of any one of paragraphs 1-70 and a pharmaceutically acceptable carrier.

72. A method of treating a proliferative disease in a subject comprising administering to the subject an SPAC of any one of paragraphs 1-70, or a pharmaceutical composition thereof.

73. The method of paragraph 72, wherein the proliferative disease is cancer.

74. The method of paragraph 73, wherein the cancer is a HER2-positive cancer.

75. The method of paragraph 73 or 74, wherein the cancer is colorectal cancer, breast cancer, stomach cancer, ovarian cancer, or esophageal cancer.

76. The method of any one of paragraphs 73-75, wherein the cancer is HER2-positive breast cancer.

77. The method of paragraph 73, wherein the cancer expresses CD38.

78. The method of paragraph 73 or 77, wherein the cancer is multiple myeloma, leukemia, or lymphoma.

79. A method of inhibiting tumor growth in a subject comprising administering to the subject an SPAC of any one of paragraphs 1-70, or a pharmaceutical composition thereof.

80. A method of delivering a stapled peptide to a cell comprising contacting the cell with an SPAC of any one of paragraphs 1-70, or a pharmaceutical composition thereof.

81. The method of paragraph 80, wherein the cell is a cancer cell.

82. The method of paragraph 80 or 81, wherein the stapled peptide has improved cellular uptake relative to a corresponding unconjugated stapled peptide.

83. A method of triggering cancer cell death comprising contacting the cancer cell with an SPAC of any one of paragraphs 1-70, or a pharmaceutical composition thereof.

84. The method of paragraph 83 for selectively killing cancer cells in the presence of non-cancer cells.

85. The method of any one of paragraphs 80-84, wherein the cell is in vitro.

86. The method of any one of paragraphs 80-84, wherein the cell is in vivo in a subject.

87. The method of any one of the preceding paragraphs, wherein the SPAC is administered intravenously.

88. The method of any one of the preceding paragraphs, wherein the subject is a human.

89. An SPAC of any one of paragraphs 1-70 for use in a method of any one of the preceding paragraphs.

90. Use of an SPAC of any one of paragraphs 1-70 for the manufacture of a medicament.

91. A kit comprising an SPAC of any one of paragraphs 1-70, or a pharmaceutical composition thereof, and optionally instructions for use.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A stapled peptide-antibody conjugate (SPAC) comprising a stapled peptide conjugated to an antibody or antigen-binding fragment thereof.

2. The SPAC of claim 1, wherein the antibody is a monoclonal antibody (mAb) or antigen-binding fragment thereof.

3. The SPAC of claim 1 or 2, wherein the antibody is an anti-cancer antibody or antigen-binding fragment thereof.

4. The SPAC of any one of claims 1-3, wherein the antibody or antigen-binding fragment thereof is directed to a target antigen expressed on a cancer cell.

5. The SPAC of any one of claims 1-4, wherein the antibody is an antibody directed against human epidermal growth factor receptor 2 (HER2), or an antigen-binding fragment thereof.

6. The SPAC of claim 5, wherein the antibody is trastuzumab.

7. The SPAC of claim 5, wherein the antibody is pertuzumab.

8. The SPAC of any one of claims 1-4, wherein the antibody is an antibody directed against CD38, CD33, CD22, TROP2, CD30, CD79b, Nectin-4, or TM4SF1, or antigen-binding fragment thereof.

9. The SPAC of any one of claims 1-4, wherein the antibody is an antibody directed against TM4SF1, or an antigen-binding fragment thereof.

10. The SPAC of any one of claims 1-4, wherein the antibody is an antibody directed against CD38, or an antigen-binding fragment thereof.

11. The SPAC of claim 10, wherein the antibody daratumumab.

12. The SPAC of any one of claim 1-11, wherein the antibody is an antibody-drug conjugate (ADC) or an antigen-binding fragment thereof.

13. The SPAC of claim 12, wherein the antibody is trastuzumab emtansine.

14. The SPAC of any one of claims 1-13, wherein the stapled peptide is a stapled anti-cancer peptide.

15. The SPAC of any one of claims 1-14, wherein the stapled peptide is a stapled antimicrobial peptide (StAMP).

16. The SPAC of any one of claims 1-15, wherein the stapled peptide is a singly stapled, doubly stapled, or stitched peptide.

17. The SPAC of any one of claims 1-16, wherein the stapled peptide is an inhibitor of a protein-protein interaction (PPI).

18. The SPAC of any one of claims 1-17, wherein the stapled peptide is an inhibitor of a BCL-2 family member protein.

19. The SPAC of claim 18, wherein the stapled peptide is an inhibitor of BCL-x_{L}, BCL-2, BCL-W, and/or MCL1.

20. The SPAC of claim 19, wherein the stapled peptide is an MCL1 inhibitor.

21. The SPAC of any one of claims 1-20, wherein the stapled peptide is an activator of a BCL-2 family member protein effector.

22. The SPAC of claim 21, wherein the stapled peptide is an activator of BAX, BAK, or BOK.

23. The SPAC of any one of claims 1-22, wherein the stapled peptide is a stapled BCL-2-interacting mediator of cell death (BIM) peptide.

24. The SPAC of any one of claims 1-23, wherein the stapled peptide is SAHB_{A1} or BIM SAHB_{A2}.

25. The SPAC of any one of claims 1-17, wherein the stapled peptide is a β-catenin inhibitor.

26. The SPAC of claim 25, wherein the stapled peptide is an inhibitor of Wnt/β-catenin signaling.

27. The SPAC of any one of claims 1-17, wherein the stapled peptide is an MDM2 inhibitor.

28. The SPAC of claim 27, wherein the stapled peptide inhibits the binding of MDM2 to p53.

29. The SPAC of claim 27 or 28, wherein the stapled peptide is ALRN-6924, ATSP-7041 or ATSP 7041 Cba10L.

30. The SPAC of claim 27 or 28, wherein the stapled peptide comprises any one of SEQ ID NOs: 161-166, or a pharmaceutically acceptable salt thereof.

31. The SPAC of any one of claims 1-30, wherein the stapled peptide triggers cancer cell death.

32. The SPAC of any one of claims 1-17, wherein the stapled peptide is a stapled Magainin peptide.

33. The SPAC of claim 32, wherein the stapled peptide is a stapled Magainin II peptide.

34. The SPAC of claim 33, wherein the stapled peptide comprises the amino acid sequence:
(SEQ ID NO: 1)
G X¹ G K F X² H S K K K F G K A X³ V G E X⁴,
or a pharmaceutically acceptable salt thereof, wherein:
X¹, X², X³, and X⁴ are amino acids;
X¹ and X² are connected via a crosslink, and X³ and X⁴ are connected via a crosslink; and
the amino acid sequence includes 0 to 9 amino acid substitutions, inclusive, at positions other than X¹, X², X³, and X⁴.

35. The SPAC of claim 34, wherein the stapled peptide comprises the amino acid sequence:
(SEQ ID NO: 2)
G X¹ G K F X² H S K K K F G K A X³ V G E X⁴ A K K,
or a pharmaceutically acceptable salt thereof, wherein the amino acid sequence includes 0 to 11 amino acid substitutions, inclusive, at positions other than X¹, X², X³, and X⁴.

36. The SPAC of claim 35, wherein the stapled peptide comprises the amino acid sequence:
(SEQ ID NO: 48)
G X¹ G K F X² Dap K K K K F G K A X³ V G E X⁴ A K
K,
or a pharmaceutically acceptable salt thereof.

37. The SPAC of claim 35, wherein the stapled peptide comprises the amino acid sequence:
(SEQ ID NO: 26)
G X¹ G Dap F X² Dap Dap Dap Dap Dap F G Dap A X³ V
GE X⁴ A Dap Dap,
(SEQ ID NO: 5)
G X¹ G K F X² K K K K K F G K A X³ V G E X⁴ K K,
(SEQ ID NO: 23)
G X¹ G K F X² H K K K K F G K A X³ V F E X⁴ A K K,
(SEQ ID NO: 24)
G X¹ G Dab F X² Dab Dab Dab Dab Dab FG Dab A X³ V
G E X⁴ A Dab Dab,
or a pharmaceutically acceptable salt thereof.

38. The SPAC of any one of claims 1-17, wherein the stapled peptide is a stapled Esculentin peptide.

39. The SPAC of claim 38, wherein the stapled peptide is a stapled esculentin-1A peptide.

40. The SPAC of claim 39, wherein the stapled peptide comprises the amino acid sequence:
(SEQ ID NO: 73)
G X¹ F S K X² K G K K I K N L X³ I S G X⁴ K G,
and pharmaceutically acceptable salts thereof, wherein:
X¹, X², X³, and X⁴ are amino acids;
X¹ and X² are connected via a crosslink, and X³ and X⁴ are connected via a crosslink; and
the amino acid sequence includes 0 to 9 amino acid substitutions, inclusive, at positions other than X¹, X², X³, and X⁴.

41. The SPAC of claim 40, wherein the stapled peptide comprises the amino acid sequence:
(SEQ ID NO: 105)
G X¹ F S K X² K G K K I K N L L X³ S G L X⁴ G,
and pharmaceutically acceptable salts thereof, wherein:
X¹, X², X³, and X⁴ are amino acids;
X¹ and X² are connected via a crosslink, and X³ and X⁴ are connected via a crosslink; and
the amino acid sequence optionally includes 0 to 8 amino acid substitutions, inclusive, at positions other than X¹, X², X³, and X⁴.

42. The SPAC of claim 40, wherein the stapled peptide comprises SEQ ID NO: 73, or a pharmaceutically acceptable salt thereof.

43. The SPAC of claim 41, wherein the stapled peptide comprises SEQ ID NO: 105, or a pharmaceutically acceptable salt thereof.

44. The SPAC of claim 41, wherein the stapled peptide comprises the amino acid sequence:
(SEQ ID NO: 113)
G X¹ F S K X² K G K K I K N L L X³ S G L X⁴ K G G
E,
or a pharmaceutically acceptable salt thereof.

45. The SPAC of any one of claims 1-44, wherein the stapled peptide is an α-helical peptide.

46. The SPAC of any one of claims 1-45, wherein the stapled peptide is 100 amino acids or fewer in length.

47. The SPAC of any one of claims 1-46, wherein the stapled peptide is 30 amino acids or fewer in length.

48. The SPAC of any one of claims 1-47, wherein the stapled peptide is conjugated to one or more stapled peptides.

49. The SPAC of claim 36, wherein the stapled peptide is of SEQ ID NO: 48, wherein X¹ and X², and X³ and X⁴, are connected via a crosslink of the following formula: 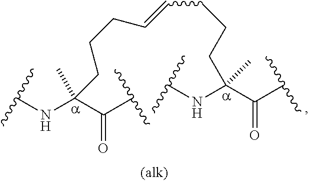 [SMILES: *N[C@@](C)(CCCC=CCCC[C@](C)(N*)C(*)=O)C(*)=O]
wherein α denotes the α-carbons of the crosslinked amino acids; and wherein the C-terminus of the stapled peptide is amidated with —NH₂.

50. The SPAC of claim 37, wherein the stapled peptide is of SEQ ID NO: 26, wherein X¹ and X², and X³ and X⁴, are connected via a crosslink of the following formula (alk); and wherein the C-terminus of the stapled peptide is amidated with —NH₂.

51. The SPAC of claim 42, wherein the stapled peptide is of SEQ ID NO: 73, wherein X¹ and X², and X³ and X⁴, are connected via a crosslink of the following formula (alk); and wherein the C-terminus of the stapled peptide is amidated with —NH₂.

52. The SPAC of claim 43, wherein the stapled peptide is of SEQ ID NO: 105, wherein X¹ and X², and X³ and X⁴, are connected via a crosslink of the following formula (alk); and wherein the C-terminus of the stapled peptide is amidated with —NH₂.

53. The SPAC of claim 44, wherein the stapled peptide is of SEQ ID NO: 113, wherein X¹ and X², and X³ and X⁴, are connected via a crosslink of the following formula (alk); and wherein the C-terminus of the stapled peptide is amidated with —NH₂.

54. The SPAC of any one of claims 1-53, wherein the antibody or antigen-binding fragment thereof is conjugated to the N-terminus of the stapled peptide.

55. The SPAC of any one of claims 1-53, wherein the antibody or antigen-binding fragment thereof is conjugated to the C-terminus of the stapled peptide via a lysine residue.

56. The SPAC of any one of claims 1-55, wherein the stapled peptide is conjugated to a cysteine residue of the antibody or antigen-binding fragment thereof.

57. The SPAC of any one of claims 1-55, wherein the stapled peptide is conjugated through a lysine residue of the antibody or antigen-binding fragment thereof.

58. The SPAC of any one of claims 1-57, wherein the antibody or antigen-binding fragment thereof is conjugated to the stapled peptide via a linker.

59. The conjugate of claim 58, wherein the linker comprises optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof.

60. The SPAC of claim 58 or 59, wherein the linker is a cleavable linker.

61. The SPAC of any one of claims 58-60, wherein the linker is pH cleavable or cleavable by a protease, esterase, or intracellular disulfide reduction.

62. The SPAC of claim 61, wherein the linker is cleavable by a protease.

63. The SPAC of any one of claims 58-62, wherein the linker is a peptidic linker.

64. The SPAC of claim 63, wherein the peptidic linker comprises —Y^{A}Y^{B}Y^{C}Y^{D}—(SEQ ID NO: 159), wherein Y^{A} is glycine, glutamic acid, or is absent; Y^{B} is valine, phenylalanine, alanine, tyrosine, or glycine; Y^{C} is citrulline, arginine, lysine, alanine, or glycine; and Y^{D} is glycine or is absent.

65. The SPAC of claim 64, wherein the peptidic linker comprises -valine-citrulline-.

66. The SPAC any one of claims 58-65, wherein the linker is of one of the following formulae: 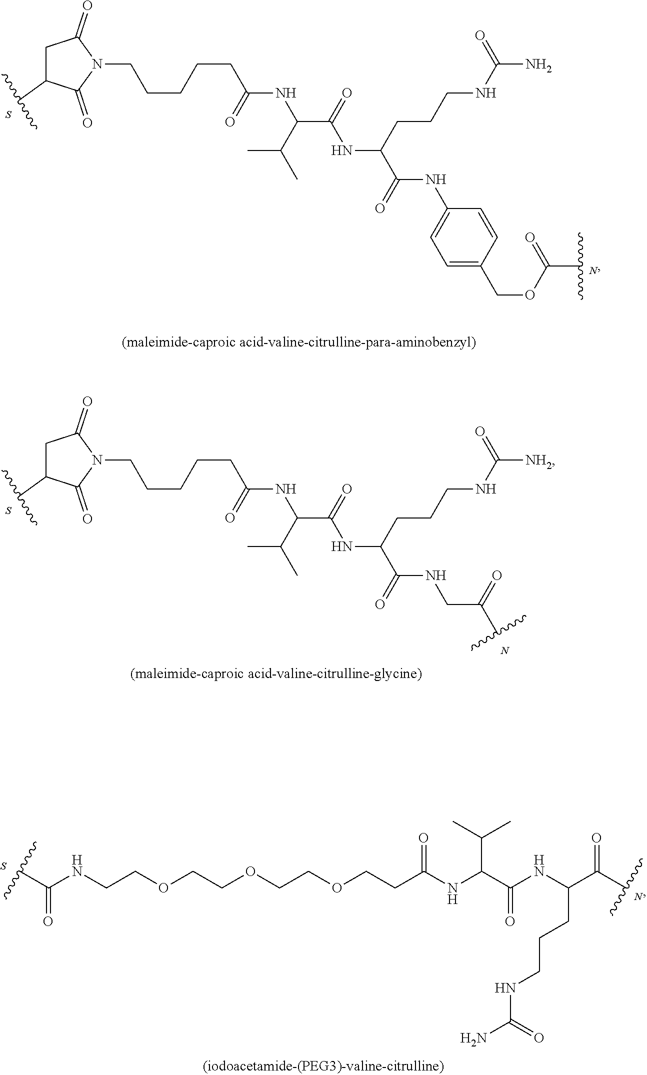 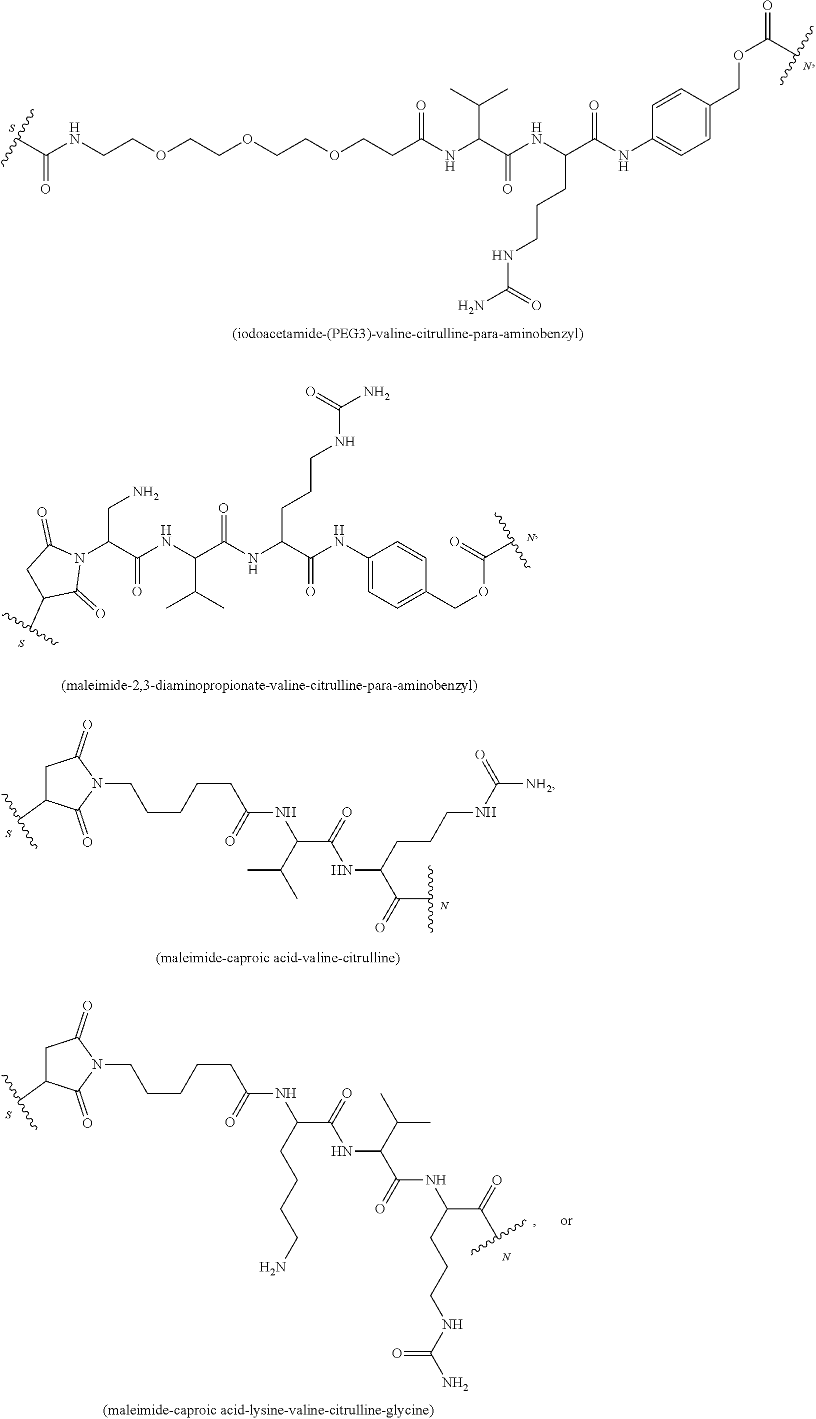 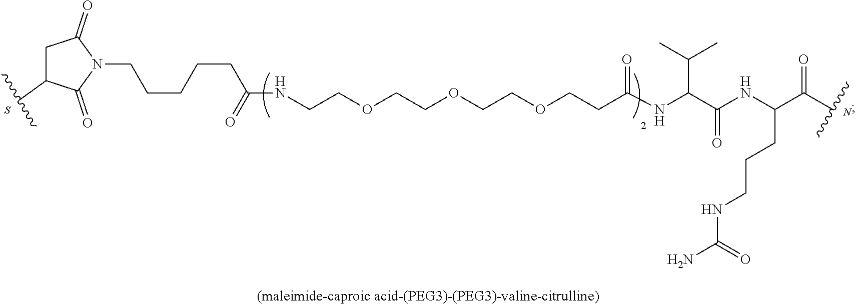
wherein S denotes the point of attachment to the antibody or antigen-binding fragment thereof; N denotes the point of attachment to the N-terminus of the stapled peptide.

67. The SPAC any one of claims 58-65, wherein the linker comprises one of the following formulae: 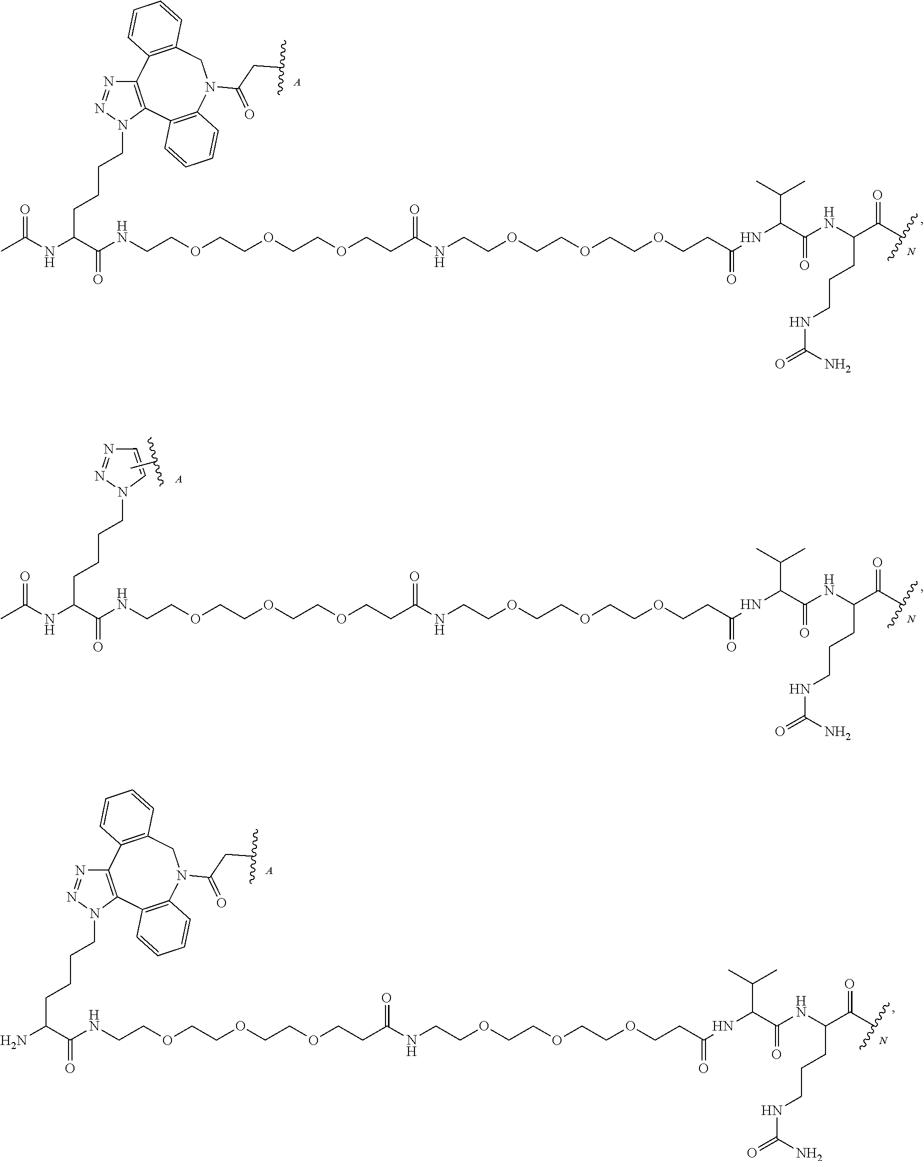 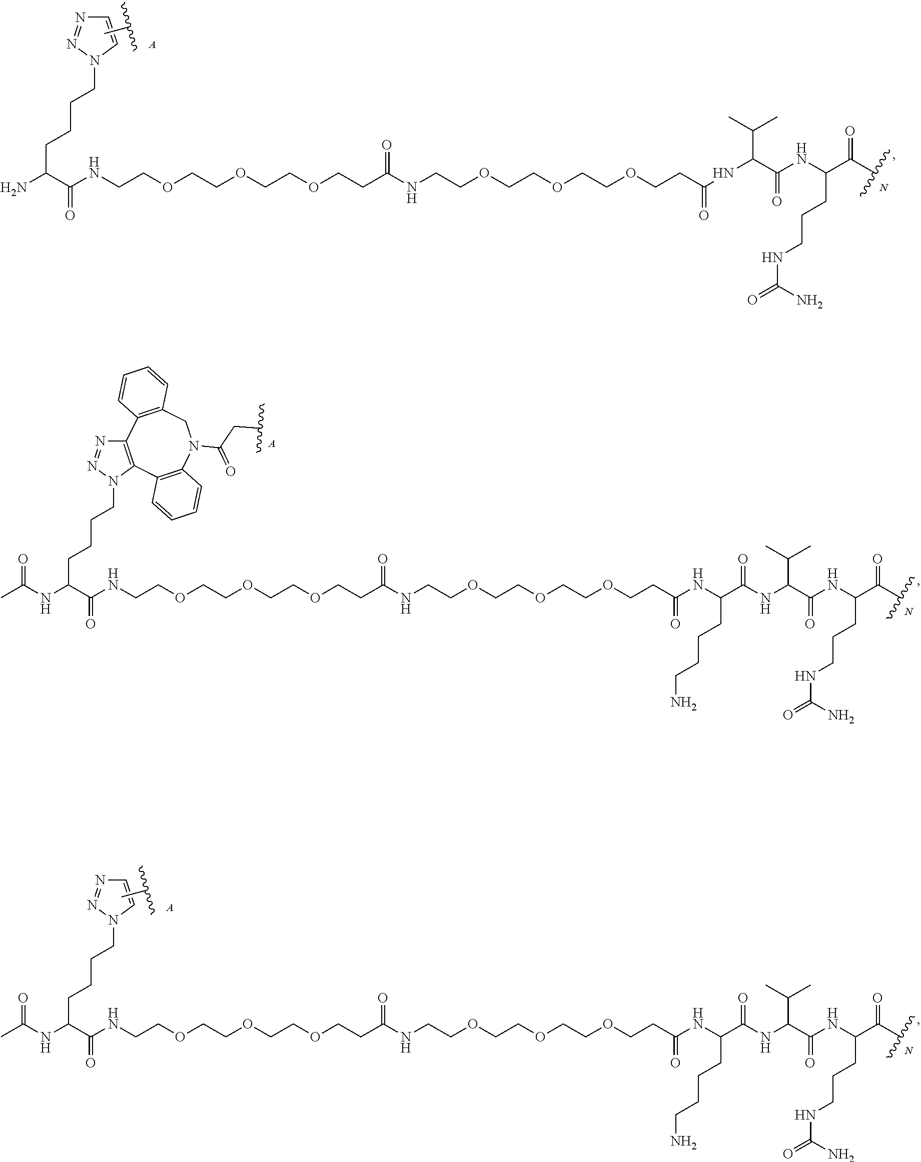 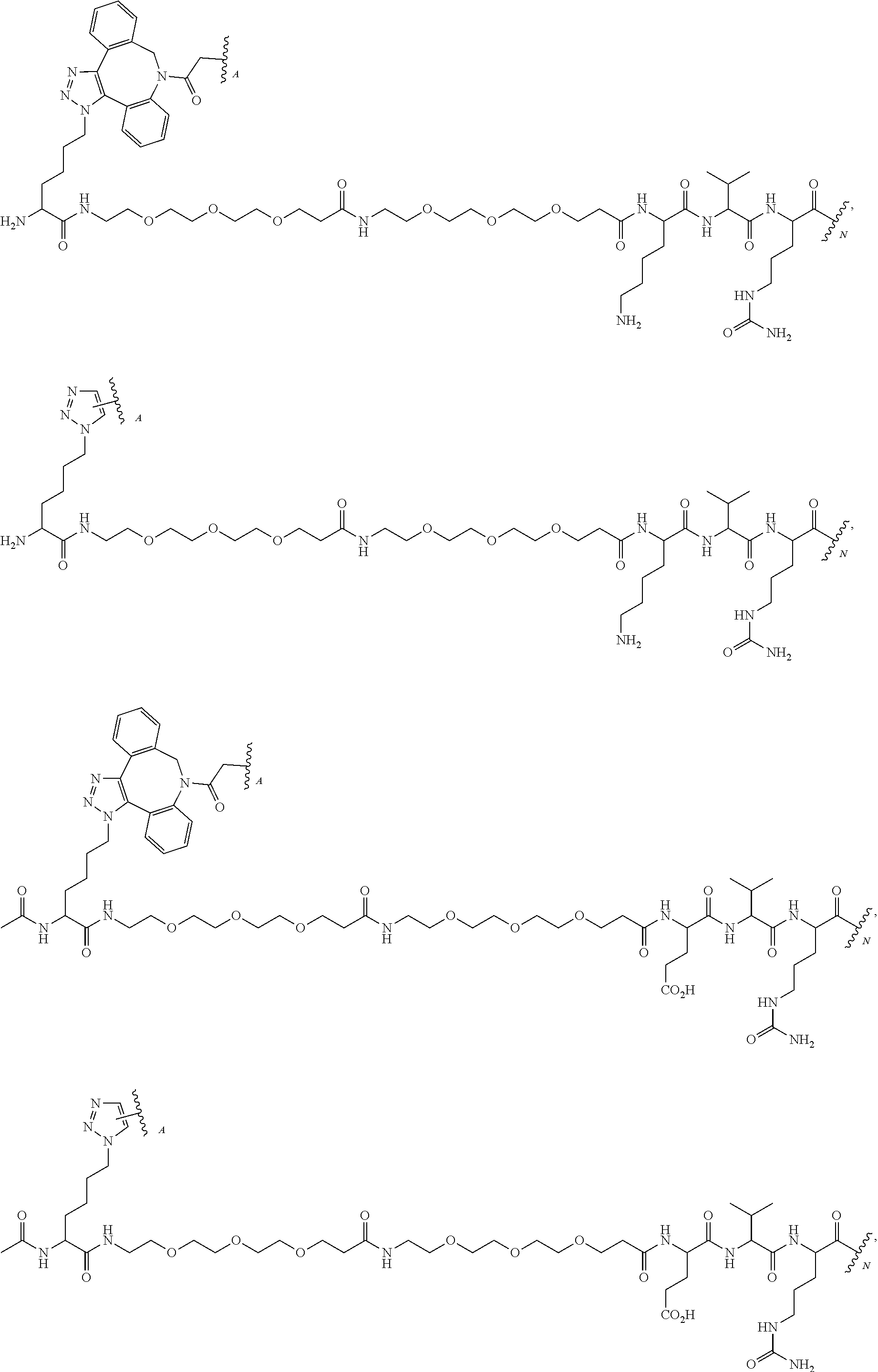 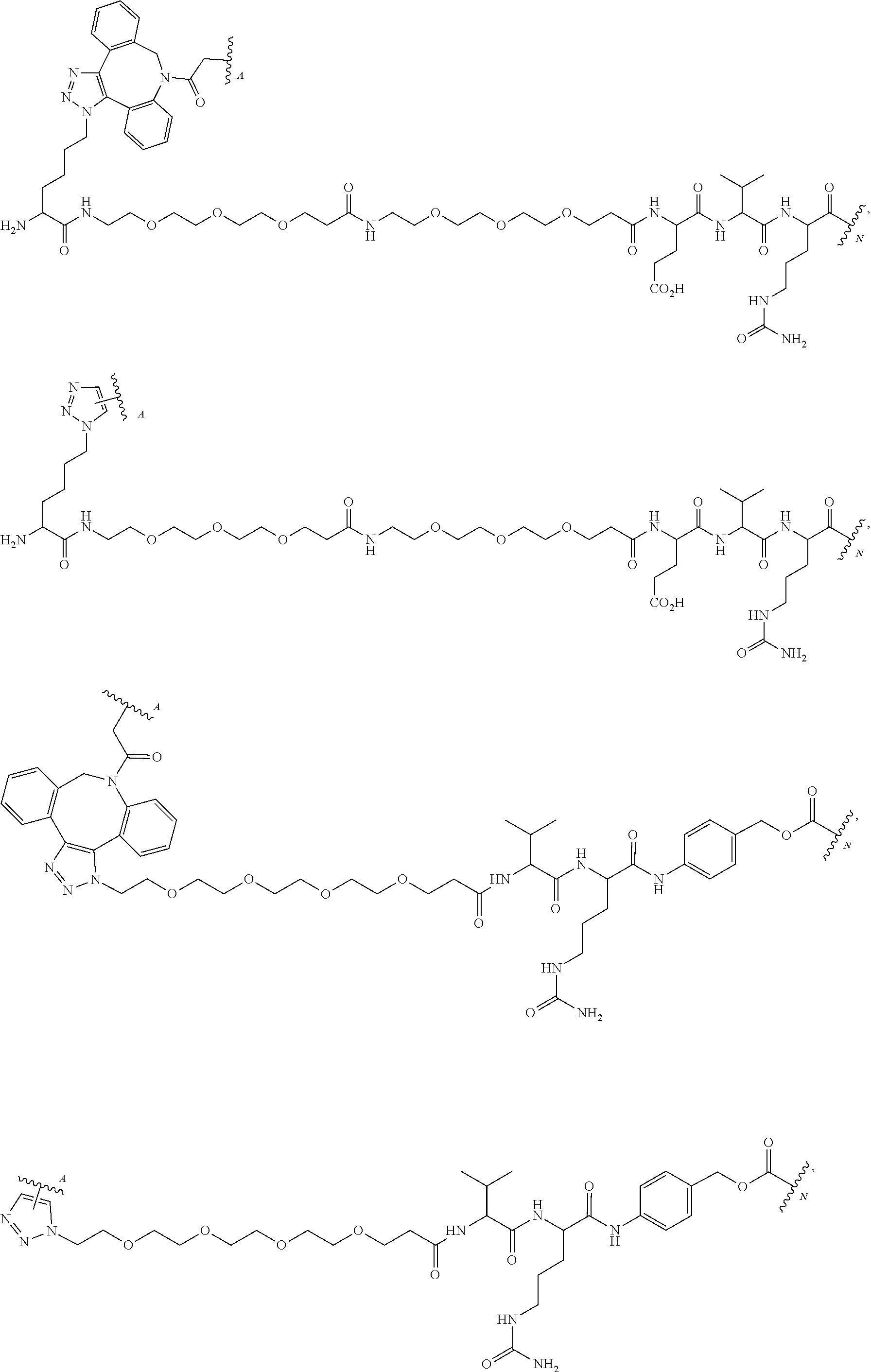 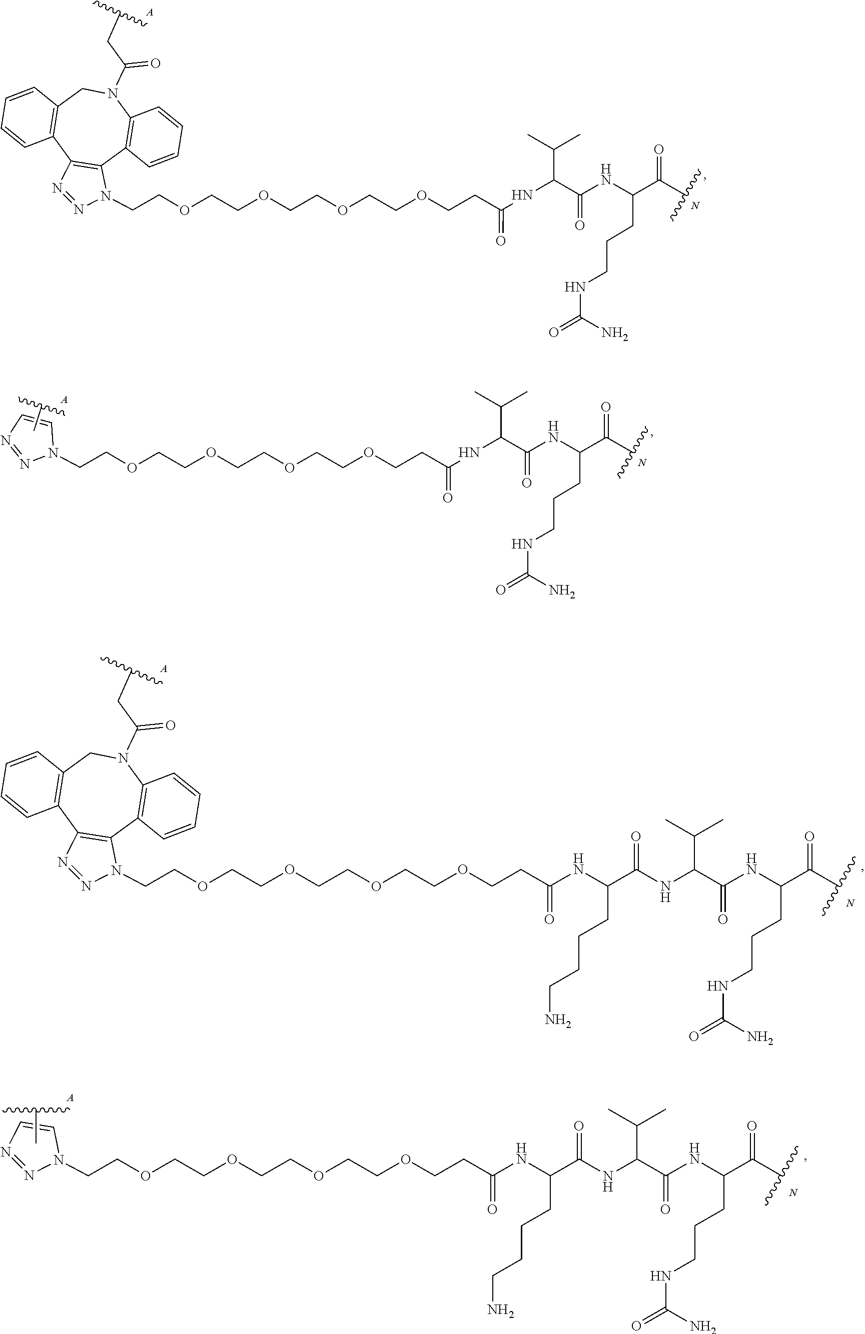 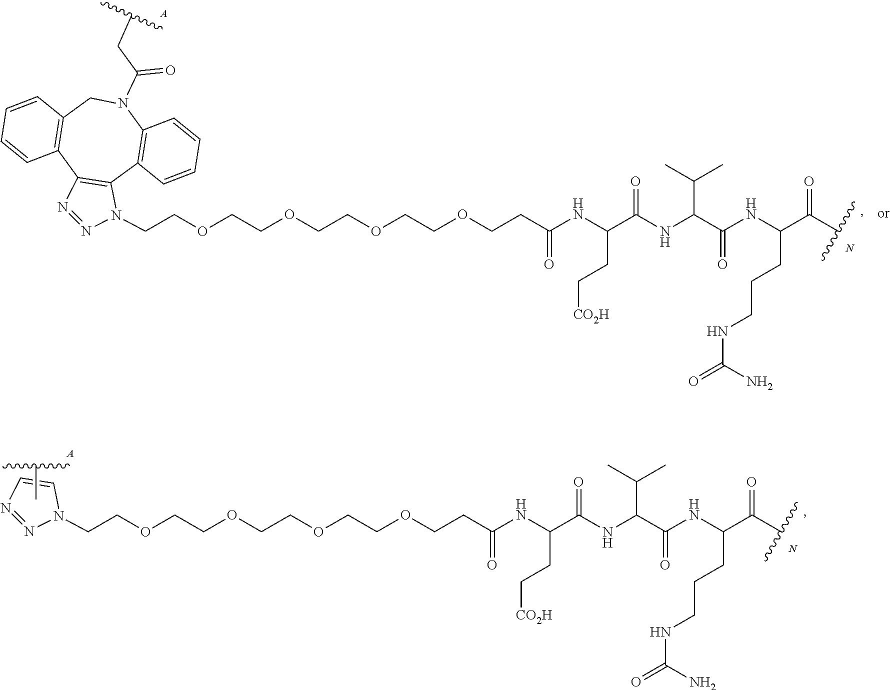
wherein A denotes a point of linkage to the antibody or antigen-binding fragment thereof; N denotes the point of attachment to the N-terminus of the stapled peptide

68. The SPAC of any one of claims 1-67, wherein the antibody or antigen-binding fragment thereof is conjugated to 2 or more stapled peptides.

69. The SPAC of any one of claims 1-68, wherein the antibody or antigen-binding fragment thereof is conjugated to 2-10 stapled peptides, include.

70. The SPAC of any one of claims 1-69 comprising an antibody or antigen-binding fragment thereof to stapled peptide ratio of about 1.8.

71. A pharmaceutical composition comprising an SPAC of any one of claims 1-70 and a pharmaceutically acceptable carrier.

72. A method of treating a proliferative disease in a subject comprising administering to the subject an SPAC of any one of claims 1-70, or a pharmaceutical composition thereof.

73. The method of claim 72, wherein the proliferative disease is cancer.

74. The method of claim 73, wherein the cancer is a HER2-positive cancer.

75. The method of claim 73 or 74, wherein the cancer is colorectal cancer, breast cancer, stomach cancer, ovarian cancer, or esophageal cancer.

76. The method of any one of claims 73-75, wherein the cancer is HER2-positive breast cancer.

77. The method of claim 73, wherein the cancer expresses CD38.

78. The method of claim 73 or 77, wherein the cancer is multiple myeloma, leukemia, or lymphoma.

79. A method of inhibiting tumor growth in a subject comprising administering to the subject an SPAC of any one of claims 1-70, or a pharmaceutical composition thereof.

80. A method of delivering a stapled peptide to a cell comprising contacting the cell with an SPAC of any one of claims 1-70, or a pharmaceutical composition thereof.

81. The method of claim 80, wherein the cell is a cancer cell.

82. The method of claim 80 or 81, wherein the stapled peptide has improved cellular uptake relative to a corresponding unconjugated stapled peptide.

83. A method of triggering cancer cell death comprising contacting the cancer cell with an SPAC of any one of claims 1-70, or a pharmaceutical composition thereof.

84. The method of claim 83 for selectively killing cancer cells in the presence of non-cancer cells.

85. The method of any one of claims 80-84, wherein the cell is in vitro.

86. The method of any one of claims 80-84, wherein the cell is in vivo in a subject.

87. The method of any one of the preceding claims, wherein the SPAC is administered intravenously.

88. The method of any one of the preceding claims, wherein the subject is a human.

89. An SPAC of any one of claims 1-70 for use in a method of any one of the preceding claims.

90. Use of an SPAC of any one of claims 1-70 for the manufacture of a medicament.

91. A kit comprising an SPAC of any one of claims 1-70, or a pharmaceutical composition thereof, and optionally instructions for use.
